(19) United States
(12) Patent Application Publication
(10) Pub. No.: US 2026/0265317 A1
(43) Pub. Date: Sep. 10, 2026

(54) NANOSTRUCTURED PROTEINS AND USES THEREOF

(71) Applicants: UNIVERSITAT AUTÒNOMA DE BARCELONA, Cerdanyola del Vallès (ES); FUNDACIÓ INSTITUT DE RECERCA DE L'HOSPITAL DE LA SANTA CREU I SANT PAU, Barcelona (ES); CENTRO DE INVESTIGACIÓN BIOMÉDICA EN RED, Madrid (ES)

(72) Inventors: Antonio VILLAVERDE CORRALES, Cerdanyola del Vallès (ES); Esther VÁZQUEZ GÓMEZ, Cerdanyola del Vallès (ES); Naroa SERNA ROMERO, Cerdanyola del Vallès (ES); Laura SÁNCHEZ GARCÍA, Cerdanyola del Vallès (ES); Ugutz UNZUETA ELORZA, Barcelona (ES); Ramón MANGUES BAFALLUY, Barcelona (ES); María Virtudes CÉSPEDES NAVARRO, Barcelona (ES); Isolda CASANOVA RIGAT, Madrid (ES)

(21) Appl. No.: 19/301,864

(22) Filed: Aug. 15, 2025

Related U.S. Application Data
(62) Division of application No. 16/611,102, filed on May 7, 2018, now abandoned.

(30) Foreign Application Priority Data
May 5, 2017 (EP) 17169722.0

(51) Int. Cl. C07K 14/42; A61K 38/00; A61P 35/00; C07K 14/21; C07K 14/34; C07K 14/47; C07K 14/52

(52) U.S. Cl. CPC C07K 14/42; A61P 35/00; C07K 14/21; C07K 14/34; C07K 14/4723; C07K 14/521; A61K 38/00; C07K 2319/10; C07K 2319/21; C07K 2319/33; C07K 2319/55; C07K 2319/60; C07K 2319/74

(57) ABSTRACT

The present invention relates to nanostructured proteins, more specifically to fusion proteins suitable for their selective delivery to specific cell and tissue types. It also relates to nanoparticles comprising such nanostructured proteins, as well as nucleic acids, vectors, cells that comprise said proteins, and the therapeutic uses thereof.

### CROSS-REFERENCE TO RELATED APPLICATIONS

The present patent application is a divisional of U.S. application Ser. No. 16/611,102, filed Nov. 5, 2019, which was a § 371 National Stage Application of International Application No. PCT/EP2018/061732, filed May 7, 2018, which claims the priority benefit of European Application No. EP 17169722.0, filed May 5, 2017.

### REFERENCE TO SEQUENCE LISTING SUBMITTED ELECTRONICALLY

The content of the electronically submitted sequence listing in XML format (Name 5489_0020002_Sequence_Listing_ST26.xml; Size: 62,552 bytes; and Date of Creation: Mar. 24, 2026) filed with the application is incorporated herein by reference in its entirety.

### FIELD OF THE INVENTION

The present invention relates to the field of nanostructured protein materials, more specifically to fusion proteins which can be used for therapy.

### BACKGROUND OF THE INVENTION

The systemic administration of drugs in form of nanoconjugates benefits from enhanced drug stability when compared to free molecules. Valuable additional properties such as cell targeting might be also merged into a given hybrid composite through the chemical incorporation of functional groups in nanoscale vehicles, taking profit from the high surface/volume ratio of nanomaterials. When administered systemically, the resulting drug loaded conjugates sizing between ~8 and 100 nm escape from renal filtration in absence of aggregation in lung or other highly vascularized organs. This fact, combined with appropriate physicochemical properties of the material might result in extended circulation time and prolonged drug exposure to target organs, thus enhancing the therapeutic impact and benefits for the patient.

Among the diversity of materials under investigation as drug carriers, that includes metals, ceramics, polymers and carbon nanotubes, proteins offer unique properties regarding biocompatibility and degradability that, in the context of rising nanotoxicological concerns, make them especially appealing.

However, many protein species are themselves, efficient drugs usable in human therapy, as attested by more than 400 protein-based products approved by main medicines agencies. Therefore, the engineering of protein drugs as self-organizing building blocks, that exhibit intrinsic therapeutic activities upon self-assembling as nanoparticles, constitutes an advantageous concept. Thus, this methodology excludes the need of further activation and drug conjugation, as the nanomaterial itself acts as a nanoscale drug (desirably between 8 and 100 nm). In that way, chemically homogenous protein nanoparticles, showing intrinsic therapeutic activities (like the current plain protein species used in human medicine—e.g, hormones, growth factors, vaccines etc.) can be biologically produced in a single step (as nanoscale assembled entities). Since the material itself acts as a drug, the possibility of drug leakage during circulation, an undesired possibility especially worrying in the case of cytotoxic agents, can be completely abolished, which becomes a significant advantage with respect to the state of the art.

The inventors previously probed into the field by applying a nanoarchitectonic principle based on the addition, to a core protein, of a cationic N-terminal domain plus a C-terminal poly-histidine. [Serna, N. et al. 2016. Nanomedicine, 12:1241-51]. It has been described in the art that these end-terminal tags and the resulting charge balance in the whole fusion promote self-assembling and oligomerization of monomeric proteins as robust toroid nanoparticles, stable in plasma [Cespedes, M. V. et al. 2014. ACS Nano., 8:4166-4176] and with high cellular penetrability if empowered with cell-targeting peptides. [Xu, Z. K. et al. 2015. Materials Letters, 154:140-3] Nonetheless, the building blocks of these protein protein structures might also contain functional peptides such as cell-targeting agents, endosomolytic agents or nuclear localization signals, in form of fused stretches with modular organization.

Therefore, to take advantage of such easy protein engineering will be highly beneficial, since a need persists in the art for drug delivery systems with enhanced selectivity and biodisponibility.

### SUMMARY OF THE INVENTION

In a first aspect, the invention relates to a fusion protein comprising
(i) a polycationic peptide,
(ii) an intervening polypeptide region and
(iii) a positively charged amino acid-rich region,
wherein the intervening polypeptide region is not a fluorescent protein alone or human p53.

In a second aspect, the invention relates to a method to prepare nanoparticles comprising multiple copies of the fusion protein according to the first aspect of the invention comprising placing a preparation of said fusion protein in a low salt buffer.

In further aspects, the invention relates to a polynucleotide encoding a fusion protein according to the first aspect of the invention, a vector comprising said polynucleotide, and a host cell comprising either said polynucleotide or said vector.

In an additional aspect, the invention relates to a nanoparticle comprising multiple copies of the fusion protein of the invention or a nanoparticle which has been obtained by the method of the invention to prepare nanoparticles.

In yet another additional aspect, the invention relates to a fusion protein, a polynucleotide, a vector, a host cell or a nanoparticle according to the invention for use in medicine.

### DESCRIPTION OF THE FIGURES

FIG. 1. Design and biochemical characterization of T22-BAK-GFP-H6 nanoparticles. A) Schematic representation of the CXCR4-binding T22-BAK-GFP-H6 building block indicating its modular composition. The amino acid sequences of the CXCR4 peptide ligand T22 and the therapeutic BH3 domain of BAK protein are shown. Lengths of the modules are here indicated as approximate. The linker sequence is GGSSRSSS. B) Mass spectrometry of the purified T22-BAK-GFP-H6 fusion indicating the experimental molecular weight (33,988.762 Da). Protein integrity is also shown through Coomassie blue-stained sodium dodecyl sulfate polyacrylamide gel electrophoresis gels (Co) and by H6 immunodetection in Western blot (WB). C) Hydrodynamic size distribution of T22-empowered nanoparticles in their native state and upon SDS-mediated disassembling. The parental BAK-GFP-H6 and GFP-H6 proteins that do not assemble and the related T22-GFP-H6 particles (and SDS-mediated disassembled monomers) are included here for size comparison. All proteins were in solution in their respective storage buffers. D) FESEM images of randomly selected fields showing the ultrastructural morphology of T22-BAKGFP-H6 nanoparticles. Bars indicate 20 nm.

FIG. 2. Cell penetrability of T22-BAK-GFP-H6 nanoparticles. A) Internalization of T22-BAK-GFP-H6 nanoparticles in cultured CXCR4+ HeLa and SW1417 cells after 24 h exposure. The intensity of intracellular fluorescence is corrected by specific fluorescence, resulting into arbitrary units (au) that are representative of protein amounts. B) Time-dependent intracellular accumulation of nanoparticles (2 μM) by Hela cells. Inset; viability of CXCR4− SW1417 cells upon exposure to 2 μM T22-BAK-GFP-H6 nanoparticles for 48 h. C) Specificity of CXCR4-mediated internalization of T22-BAK-GFP-H6 nanoparticles determined by the use of the CXCR4+ inhibitor AMD3100.

FIG. 3. Accumulation and organ biodistribution of T22-GFP-H6 and T22-BAK-GFP-H6 nanoparticles and unassembled BAK-GFP-H6 protein in CXCR4+ colorectal tumors. A) Representative ex vivo tumor fluorescence images (FLI) at 2, 5, 24 and 48 h after iv administration of 330 μg dose of each protein. B) GFP fluorescence quantitation in tumors at 2, 5, 24 and 48 h using the IVIS spectrum system. The FLI ratio was calculated dividing the GFP signal from the protein-treated mice by the auto-fluorescent signal of buffer-treated mice at each organ. & and #p<0.05 bars indicate statistically significant compared to the rest of T22-BAK-GFP-H6-treated groups. *p<0.05 bars indicate a statistically significant between the designated groups. C) Immunohistochemistry against the His-tag domain of the nanoparticles in the tumors at 5 h. D) Representative ex vivo images of the material accumulated in mouse brain, lung, heart, liver, kidney and bone marrow tissues after treatment. Note the absence or residual fluorescence in these organs compared to tumors. E) Representative H& E staining showed no altered architecture in any of the organs. Abbreviations: H&E, Hematoxylin and eosin staining; iv: intravenous; FLI: Fluorescent imaging; NP: nanoparticle.

FIG. 4. Reduced proliferation index, caspase-3 activation, proteolyzed PARP, apoptosis induction and necrotic rates in tumors bearing mice at 2, 5, 24 and 48 h after administration of T22-BAK-GFP-H6 compared to buffer and T22-GFP-H6 and BAK-GFP-H6 control counterparts. Quantitation in tumors of the number of mitotic figures (mitotic activity index) by H&E staining (A) and both, cleaved (active) caspase-3 (B) and proteolyzed PARP (C) positive tumor cells by IHC. #, & p<0.05 bars indicate statistically significant compared to T22-BAK-GFP-H6-treated groups at each time; * p<0.05 bars indicate statistically significant between the designated groups. D) Counts of apoptotic figures detected by nuclear condensation after Hoechst staining. *p<0.05 bars indicate statistically significant between the designated groups. E) Measurements of total and necrotic area (μm²) in low-power field magnification tumor slices using the Cell D software. *p<0.05 bars indicate statistically significant between 2 and 5 h-treated groups. All quantified values in panels A-D were obtained by counting 10 high-power field (×400) per sample. Data was expressed as mean±SE. All statistical analyses were performed applying the Mann Whitney U-test. Abbreviations: H&E, Hematoxylin and eosin staining.

FIG. 5. Physical and biological characterization of T22-PUMA-GFP-H6 and T22-GWH1-GFP-H6 nanoparticles. Schematic representation of the building blocks based on PUMA (A) and on GWH1 (B). The amino acid sequences of the therapeutic protein stretches are indicated while the rest of the constructs are as in FIG. 1. The DLS plots of the nanoparticles (green) and of the disassembled building blocks (red) are depicted, sided by the value of the peaks in nm. Representative FESEM images of isolated nanoparticles are also shown. Bars indicate 40 nm. C) Representative ex vivo tumor fluorescence images (FLI) and normal organs (brain, kidney, lung, heart and liver tissues) after iv administration of 300 μg dose of each nanoparticle at 5 h. D) Quantitation of fluorescence signal (radiant efficiency) in the respective organs. E and F) Quantitation of the number of mitotic figures, (H&E staining), the number of apoptotic figures detected by nuclear condensation after Hoechst staining and necrosis area (H&E staining) in tumors at 5 after the administration of T22-PUMA-GFP-H6 or T22-GWH1-GFP-H6 compared to T22-GFP-H6 control counterpart. Quantitations were done as indicated in FIG. 4.

FIG. 6: Characterization by DLS of the H6-GFP-R9 and of the H6-R9-GFP proteins Hydrodynamic size distribution of H6-GFP-R9 and H6-R9-GFP nanoparticles determined by DLS in three independent determinations.

FIG. 7: Characterization of GWH1-based protein nanoparticles. A). Schematic representation of recombinant proteins used in this study. Length of boxes are only representative. T22-GFP-H6 [13] and T22-GWH1-GFP-H6 (Serna et al, submitted) have been fully described elsewhere. B) Mass spectroscopy analysis of recombinant GWH1-based proteins, upon affinity chromatography. C) Visualization of purified proteins through TGX gel chemistry upon PAGE. D). Size of GWH1-GFP-H6 nanoparticles compared to the parental GFP-H6. The size of T22-GWH1-GFP-H6 nanoparticles is 24.6 nm, and it will be fully described elsewhere (Serna et al, submitted). E). FESEM imaging of purified GWH1-GFP-H6 nanoparticles at different magnifications. The bars indicate 50 nm.

FIG. 8: Antibacterial activities of GWH1-based protein nanoparticles. A) Cell viability of different bacterial species exposed to 1.25 mg/ml GWH1-based protein nanoparticles for 24 h (48 h for M. luteus). T22-GFP-H6 is included as negative control. B) Dose-dependent antibacterial activity of GWH1-GFP-H6 upon incubation for 24 h (48 h for M. luteus). C) Bacterial cell lysis upon incubation with 1.25 mg/ml GWH1-GFP-H6 nanoparticles for 24 h (48 h for M. luteus) monitored by light microscopy.

FIG. 9: Cytotoxic activities of GWH1-based protein nanoparticles. A) Protein internalization monitored through intracellular GFP fluorescence 24 h after exposure to nanoparticles. Data has been corrected by specific fluorescence values to allow comparison in a molar basis. B) HeLa cell viability upon exposure to 10 μM of protein nanoparticles for 24 h. C) Light microscopy of cultured Hela cells exposed to protein nanoparticles upon conditions of panel B.

FIG. 10: Design of the T22-DITOX-H6 and T22-PE24-H6 nanoparticles A. Native structure of A-B toxins such as diphtheria toxin (Corynebacterium diphtheriae) or exotoxin A (Pseudomonas aeruginosa). The native toxin is divided in two fragments (A and B). Fragment A includes the catalytic domain (C-domain), whereas the fragment B comprises the translocation and the receptor binding domain (T- and R-domain). The selected domains for the construction of the recombinant nanoparticles are coloured in dark purple (T22-PE24-H6 construct does not include the T-domain). B. Modular organization of T22-DITOX-H6 and T22-PE24-H6, in which T22 acts as both CXCR4 ligand and as an architectonic tag. Functional segments are intersected by linker regions (light blue) and furin-cleavage sites (dark blue, boldface). A natural furin-cleavage site also occurs within DITOX (dark blue, underlined), that separates the amino terminal catalytic domain from the carboxy terminal translocation domain. A KDEL peptide has been incorporated neighboring the H6 region in T22-PE24-H6. Box sizes are only indicative. Two additional proteins, namely T22-DITOX-H6 F- and T22-PE24-H6 F-were constructed for comparative purposes, precisely lacking the engineered furin cleavage sites (boldface dark blue regions). C. Expected pathway for the cytotoxicity of T22-DITOX-H6 and T22-PE24-H6 nanoparticles over CXCR4+ target cells, upon intracellular furin-mediated release of protein domains useful for biodistribution and cell penetration steps but irrelevant for cell killing. Color images on request.

FIG. 11: Nanoarchitecture of toxin-based proteins T22-DITOX-H6 and T22-PE24-H6. A. Size and SDS-mediated disassembling of T22-DITOX-H6 and T22-PE24-H6 nanoparticles determined by DLS. Values of peak sizes (mode) are indicated in bold (in nm, ±SEM). Zpotential (Zp) values of the nanoparticles are also indicated. The molecular mass of proteins upon purification is shown by Western Blot upon PAGE-SDS. B. FESEM examination of purified T22-DITOX-H6 and T22-PE24-H6 materials. Bars indicate 50 nm. Color images on request.

FIG. 12: Internalization of toxin-based nanoparticles in CXCR4+ cells. A. Mass spectroscopy of pure unlabeled and ATTO-labelled (*) T22-DITOX-H6 and T22-PE24-H6 proteins. B. Dose dependent uptake of T22-DITOX-H6* and T22-PE24-H6* nanoparticles in CXCR4+ HeLa cells upon 1 h of exposure. C. Time course kinetics of cell internalization of T22-DITOX-H6* and
T22-PE24-H6* nanoparticles (1 μM) in CXCR4+ HeLa cells. Note the short error bars in the plot. D. Protein (100 nM) uptake inhibition by the CXCR4 antagonist AMD3100 (+) upon 1 h of exposure. Significant differences between relevant data pairs are indicated as § for p<0.01. All A, B and C data are presented as mean±SEM (n=2). E. Confocal microscopy of HeLa cells exposed for 5 h to T22-DITOX-H6* and T22-PE24-H6* nanoparticles (1 μM). The Cell Mask membrane staining (red) was added together with nanoparticles to observe the endosomal membrane. Nanoparticles are visualized in green and nuclear regions in blue. The yellow spots indicate merging of red and green signals. In the insets, 3D Imaris reconstructions of confocal stacks. Bars indicate 5 m. Color images on request.

FIG. 13: Specific cytotoxicity of toxin-based nanoparticles in CXCR4+ cells. A. Detection of intracellular T22-DITOX-H6 by Western blot analysis of HeLa cell extracts, after exposure of the
cell cultures to nanoparticles (1 μM protein) for 24 h. M indicates the migration of molecular weight markers. B. Left: Cell death induced by T22-DITOX-H6 and T22-PE24-H6 nanoparticles
(10 nM) over a SW1417 CXCR4− cell line and different CXCR4+ cell lines (including an isogenic, CXCR4+SW1417 version), 48 h after exposure (72 h for SW1417 cell line). Significant differences between relevant data pairs are indicated as ¥ 0.01<p<0.05 and § p<0.01. Right: inhibition of HeLa cell death (induced by 10 nM of protein nanoparticles) by either the CXCR4 antagonist AMD3100 or by 2 μM protein T22-GFP-H6. Significant differences between relevant data are indicated as a change in the letter, from “a” to “b”. All the significant results were p<0.01. All data are presented as mean±SEM (n=3). C. HeLa cell death promoted by T22-DITOX-H6 F- and by T22-PE24-H6 F-, compared to the related T22-DITOX-H6 and by T22-PE24-H6 respectively. Cells were exposed to 10 nM of each protein for 48 h. Data and statistics are as in panel B. D. Immunocytochemistry staining showing the lack of CXCR4 expression in the isogenic SW1417 CXCR4− cells as compared with the high CXCR4 expression in SW1417 CXCR4+ cells. Bar indicates 50 m. E. Differential CXCR4 protein expression in these cells assessed by an immunoblotting assay. Glyceraldehyde-3-phosphate dehydrogenase (GADPH) was used as protein loading control. Color images on request.

FIG. 14: Biodistribution kinetics of T22-DITOX-H6* and T22-PE24-H6* nanoparticles in a CXCR4+ colorectal cancer mouse model. Ex vivo fluorescence emitted by subcutaneous tumor and relevant organs in buffer-administered (control) and T22-DITOX-H6*- and T22-PE24-H6*-treated mice at 5, 24, 48 and 72 h after 50 μg or 300 μg single dose i.v. administration. Emission scales are shown as radiant efficiency units (see materials and methods regarding the protein nanoparticles based on Diphteria toxin (DITOX) and Pseudomonas aeruginosa exotoxin (PE24)).

FIG. 15: Local induction of apoptosis in tumor by ATTO-labelled and unlabeled T22-DITOX-H6 (50 μg) and T22-PE24-H6 (300 μg) nanoparticles. A. Representative H&E staining of
subcutaneous tumors showing apoptotic figures (black arrows). No significant apoptosis was detected in liver tissue at the studied times. Few and small inflammation foci in this organ were observed and are indicated by yellow arrows, which were resolved at 72 h, returning to histologically normal parenchyma. Note the absence of histological alterations in kidneys. Bar: 50 am. B. Number of apoptotic cell bodies in H&E tumor slices per ten high-power fields (400× magnification) are plotted for each nanoparticle. For the experimental times showing higher number of apoptotic lesions we also show representative Hoechst staining of subcutaneous tumors in animals treated with unlabeled protein versions, at different magnifications. All data are presented as mean SEM (n=3). Statistical significance: ^{a}p=0.008; ^{b}p=0.027: ^{c}p=0.010; ^{d,e,f}p=0.001.

FIG. 16: Pharmacokinetics, antitumoral effect and mouse body weight after T22-DITOX-H6 and T22-PE24-H6 administration. A. Pharmacokinetics of T22-DITOX-H6* and T22-PE24-H6* after a 50 μg or 300 μg intravenous bolus administration respectively. Fluorescence was recorded in plasma obtained after blood centrifugation at time 0, 1, 2, 5, 24 and 48 h (n=3 per time point). B. Antitumor effect of T22-DITOX-H6 and T22-PE24-H6 measured by the analysis of tumor volume and number of apoptotic bodies at the end of the experiment, after repeated dose administration for each nanoparticle (10 μg, three times a week, ×8 doses). C. Evolution of mouse body weight after the described repeated dose regime for the protein nanoparticles. Statistics are ¥ for 0.01<p<0.05 and § for p<0.01. All data are presented as mean±SEM, n=3.

FIG. 17: Physicochemical properties of T22-mRTA-H6. A. Modular scheme and amino acid sequence of T22-mRTA-H6. mRTA is the modified fragment A of ricin, described in material and methods, in which the Asn residue 132 has been replaced by Ala (underlined). Sizes of the boxes are only indicative. B. Fractioning between insoluble (I) and soluble (S) cell fractions in total cell extracts, revealed by WB, upon protein production at 37° C. for 3 h. SDS-PAGE analysis of T22-mRTA-H6 upon one-step affinity purification, revealed by Coomassie blue (CB) staining and by Western blot (WB) using an anti-his antibody. U and AB stand for Unstained and All Blue markers respectively (Bio-Rad, Refs161-0363 and 161-0373), and 1, 2 and 3 indicate, respectively, the unspecific elution peak and two peaks with increasing level of purity. Protein in peak 3 was used in further experiments. C. Hydrodynamic size (and Z potential) of T22-mRTA-H6 nanoparticles formed spontaneously upon purification (red line), determined by DLS. Pdi is polydispersion index, and all figures indicate nm. The size of the monomer, determined upon disassembling the material with 1% SDS for 40 min, is also indicated (green line). D. FESEM imaging, at different magnifications, of T22-mRTA-H6 nanoparticles. Bars represent 20 nm. E. Far UV CD of T22-mRTA-H6 in carbonate-bicarbonate buffer at pH 8 measured at 25° C. F. ThT fluorescence emission spectra alone (black line) or in the presence of T22-mRTA-H6 (light grey line) and T22-mRTA-H6
previously heated at 100° C. (dark grey line). λex=450 nm. In the plot at the bottom, ThT fluorescence emission at 490 nm of T22-mRTA-H6 (black bar) and T22-mRTA-H6 previously heated at 100° C. (grey bars). GF. Size of T22-mRTA-H6 nanoparticles dialyzed against 51 mM sodium phosphate, 158.6 mM trehalose dehydrate, 0.01% polysorbate-20 buffer at different pH values, determined by DLS. Color images on request.

FIG. 18: Cytotoxicity and CXCR4 specificity of T22-mRTA-H6 nanoparticles. A. Viability of cultured CXCR4+ HeLa cells upon 72 h of exposure to T22-mRTA-H6 nanoparticles at different concentrations, presented as a dose-response curve. B. Inhibition of cell death in HeLa cells exposed to different concentrations of T22-mRTA-H6 nanoparticles for 72 h, mediated by the CXCR4 antagonist AMD3100 (always at an excess molar ratio of 10:1). C. Levels of CXCR4 membrane protein determined by flow cytometry of different cell lines (3T3, MV411, THP1 and HeLa), expressed as mean fluorescence intensity ratio±SE. D. Extent of internalization of 100 nM T22-GFP-H6 in the different cell lines at 172 h of exposure. Results are expressed as mean fluorescence intensity ratio±SE. EC. Viability of cultured CXCR4− 3T3 cells upon 4872 h of exposure to T22-mRTA-H6 nanoparticles and the small molecular weight antitumoral drug Ara-C, at different concentrations. The commercial CXCR4− and CXCR4+ human AML cell lines (MV411 and THP1 respectively) are included as controls. Ara-C showed cytotoxicity above 100 nM (not shown). The standard error is represented in all bars. The level of significance is indicated by superscripts (*p<0.05, **p<0.01).

FIG. 19: Cell penetrability and intracellular toxicity of T22-mRTA-H6 nanoparticles. A. Intracellular fluorescence in cultured HeLa cells exposed to 100 nM of ATTO488 stained T22-mRTA-H6. Extracellular fluorescence was fully removed by a hash trypsin treatment as described (Richard, J. P. et al., The Journal of biological chemistry 2003, 278 (1):585). B. Under the same conditions, the externalized phosphatidylserine was detected by Annexin V Detection Kit (APC, eBioscience) in cells exposed to non-stained T22-mRTA-H6. Dead cells were spotted with propidium iodide (PI). Quadrant Q1 shows HeLa cells marked with PI. Q2 shows cells marked with Annexin V and PI. Q3 shows cells without PI nor Annexin V. Q4 shows cells marked with Annexin V. Therefore, dead cells are shown in Q1 and Q2 while living cells in Q3 and Q4. Apoptotic cells are shown in Q4. At the bottom, Hoechst staining of HeLa cell under the above conditions. Images were obtained by fluorescence microscopy (×400). C. Loss of JC-1 Red fluorescence in T22-mRTA-H6-treated cells as described above, indicative of a change in the mitochondrial Δψ. D. Levels of cellular ROS detected with a fluorescence microplate assay. HeLa cells were treated with either buffer, T22-mRTA-H6 (100 nM, for 15 or 24 hours) or 100 μM Pyocyanin (1 hour) as a positive control. Values are expressed as relative fluorescence units±SE. E. Inhibition of caspases with zVAD-fmk reverses the antitumor activity of T22-mRTA-H6 in HeLa cells. Cells were pretreated for 1 hour with 100 μM zVAD-fmk and then exposed to 100 nM T22-mRTA-H6 for 48 hours. Cell viability is expressed as the percentage of cell survival compared with the control. Values are mean±SE. Vehicle indicates treatment with buffer. The level of significance is indicated (*p<0.05, **p<0.01).

FIG. 20: Antitumor activity of T22-mRTA-H6 in a disseminated AML mouse model. A. Follow-up of bioluminescence emitted by mice treated with soluble T22-mRTA-H6 nanoparticles (T22mRTA), T22-mRTA-H6 IBs (IB-T22mRTA) or buffer (VEHICLE) during the 14 days of the experiment, analyzed by IVIS Spectrum. B. Levels of luminescence detected ex vivo in IVIS Spectrum in the tissues infiltrated with leukemic cells such as backbone, hindlimbs, liver and spleen of mice treated with buffer (VEHICLE), T22-mRTAH6 IB (IB-T22mRTA) or soluble T22-mRTA-H6 (T22mRTA). C. Detection of CD45 positive cells by IHQ in spleen, liver and bone marrow of mice treated with buffer (VEHICLE), T22-mRTA-H6 IBs (IB-T22mRTA) or soluble T22-mRTA-H6 nanoparticles (T22mRTA). T22mRTA, mouse treated with soluble T22-mRTA-H6; IB-T22mRTA, mouse group treated with T22-mRTA-H6 IBs; VEHICLE, group treated with vehicle. Bars indicate 50 m. Color images on request.

FIG. 21: Histopathology in the disseminated AML mouse model after a treatment with T22-mRTA-H6. Hematoxylin and eosin staining of normal (heart, lung, kidney) and leukemia infiltrated organs (bone marrow, liver, spleen). Images were taken in the microscope with a 20× objective and an Olympus DP72 digital camera. H&E, Hematoxylin and Eosin; T22mRTA, mouse treated with soluble T22-mRTA-H6; IB-T22mRTA, mouse group treated with T22-mRTA-H6 IBs; VEHICLE, mouse group treated with buffer. Bars indicate 50 m. Color images on request.

### DETAILED DESCRIPTION OF THE INVENTION

The authors of the present invention have observed that a fusion protein comprising a polycationic peptide and an positively charged amino acid-rich region flanking a biologically active intervening polypeptide are capable of being assembled into nanoparticles wherein the activity of the is biologically active intervening polypeptide is preserved. These nanoparticles can be delivered to specific cells by virtue of the affinity between the polycationic region and cell-surface receptors, thereby allowing the specific delivery of the biologically active polypeptide to the cell of interest.

While fusion proteins having similar structure and wherein the intervening polypeptide are fluorescent proteins have been described in the art, the results obtained by the inventors are unexpected due to the essentially different mechanisms involved in the biological activity of fluorescent proteins on one hand and proapoptotic peptides, cytotoxic proteins and other therapeutic polypeptides that might execute a healing activity in cancer or other pathologies on the other hand.

In the case of GFP and other fluorescent proteins, these are biologically active (fluorescence emission) by an intrinsic activity (a proper folding and conformational structure of the fluorophore) that does not require interaction with or involvement of any external factor. The protein is active per se in absence of any cell or cell structure.

However, proapoptotic peptides, cytotoxic proteins and other therapeutic polypeptides that might execute a healing activity in cancer or other pathologies do require complex interactions with cell structures and cell proteins that allows reaching a proper cellular compartment (membrane crossing etc) at a concentration above a specific threshold (different among diverse therapeutic agents) capable of triggering death of the target cells through complex signaling and metabolic cascades.

That means that it is not obvious or predictable that a functional protein other than a fluorescent protein might remain biologically active and show therapeutic activity in vivo in a nanostructured form, and that such complex spectrum of activities based on specific protein-protein interactions can be conserved. The activity of cytotoxic or pro-apoptotic proteins is dependent on living cells and on a correct performance in a complex intracellular cell environment.

It cannot be predicted or expected that a cytotoxic protein, organized as an oligomeric nanostructure, will keep intact the whole interactome and biological activity to execute its therapeutic function.

On the other hand, it is also not predictable than a protein other than GFP, can be efficiently produced in soluble form, and able to form nanoparticles, stable, targeted and lacking any side-interactivity that would affect the desired biodistribution in vivo, in the diseased tissue or intracellularly.

Moreover the inventors have also generated nanostructured versions of toxins in which a protein toxin fragment is produced in bacteria flanked by a polycationic peptide (such a the T22 peptide) and a positively charged amino acid-rich region (for instance a polyhistidine residue). These toxins have been the Pseudomonas aeruginosa exotoxin, the diphtheria toxin (both from bacteria) and the plant toxin ricin. All these toxins irreversibly inhibit protein synthesis by acting as “ribosome-inactivating proteins” (RIPs), being among the most potent cytotoxic proteins in nature (specially ricin). These fusion proteins further comprise a protease cleavage site (for instance, the furin cleavage site) so that the protein is cleaved in the endosomes and released in its active toxin form with few additional amino acids, during the endosomal escape. The design is aimed to release in the cytoplasm of the target cell, the most ‘natural’ version as possible of the active form. The results obtained by the inventors using the bacterial toxin-containing fusion proteins are also completely unexpected because it was not predictable in advance if:
the selected segments of the toxins would be active as fusion proteins,
they would be produced in bacteria in soluble form and self-assemble,
they would be still active as regular oligomeric nanoparticles,
the nanoparticles would be stable and selective in systemic administration,
the protease active site would be active in this particular accommodation site,
the protease cleavage would allow the cytotoxic action of the resulting toxin segment
the active toxin segment would reach its target inside the cell for a proper interaction and ribosomal inactivation

### Fusion Proteins of the Invention

In a first aspect, the invention relates to a fusion protein comprising
(i) a polycationic peptide,
(ii) an intervening polypeptide region and
(iii) a positively charged amino acid-rich region,
wherein the intervening polypeptide region is not a fluorescent protein alone or human p53.

The term “fusion protein” is well known in the art, referring to a single polypeptide chain artificially designed which comprises two or more sequences from different origins, natural and/or artificial. The fusion protein, per definition, is never found in nature as such.

The term “single polypeptide chain”, as used herein means that the polypeptide components of the fusion protein can be conjugated end-to-end but also may include one or more optional peptide or polypeptide “linkers” or “spacers” intercalated between them, linked by a covalent bond.

The term “peptide” or “polypeptide”, as used herein, generally refers to a linear chain of around 2 to 40 amino acid residues joined together with peptide bonds. It will be understood that the terms “peptide bond”, “peptide”, “polypeptide” and protein are known to the person skilled in the art. From here on, “peptide” and “polypeptide” will be used indistinctly.

As used herein, an “amino acid residue” refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

### A. The Polycationic Peptide

The term “polycationic peptide” or “first positively charged amino acid-rich region” as used herein, corresponds to a polypeptide sequence containing multiple positively charged amino acids. The polycationic peptide may be formed exclusively by positively charged amino acids or may contain other amino acids provided that the overall net charge of the region at pH 7 is positive.

It is well known in the art that amino acids and their corresponding amino acid residues possess different properties depending on their side chains and they may be grouped depending on those properties. Thus, at physiological pH, five amino acids show an electrical charge: arginine, histidine, and lysine are positively charged while aspartic acid and glutamic acid are negatively charged. The person skilled in the art will acknowledge then that the polycationic peptide of the invention corresponds to a polypeptide with a net electrical charge of more than one positive charge in physiological pH conditions. Accordingly, the polycationic peptide of the invention is not limited by the presence of one or more negatively charge amino acid residues as long as there are always enough positively charged amino acid residues to result in a net positive electrical charge of two or more.

Thus, in one embodiment of the invention, the polycationic peptide of the invention is selected from the group consisting of
(i) an arginine-rich sequence,
(ii) a sequence which is capable of specifically interacting with a receptor on a cell surface and promoting internalization of the fusion protein on said cell,
(iii) the GW-H1 peptide,
(iv) a CD44 ligand,
(v) a peptide capable of crossing the blood-brain barrier,
(vi) a cell penetrating peptide and
(vii) a nucleolin-binding peptide.

### (i) Arginine-Rich Sequence

As aforementioned, the arginine amino acid and its residue present positive charge at physiological pH. It will be understood that an “arginine-rich sequence” refers to a polypeptide sequence containing multiple arginine residues. Thus, the polypeptide sequence may comprise 33%, preferably 40%, preferably 45%, preferably 50%, preferably 55%, preferably 60%, preferably 65%, preferably 70%, preferably 75%, preferably 80%, preferably 85%, more preferably 90%, more preferably 95%, even more preferably 99%, yet even more preferably 100% of the amino acid residues of its complete sequence as arginine residues. It will be understood that whenever the sequence of the arginine-rich sequence comprises less than the 100% of the sequence as arginine residues, these residues do not need to be all adjacent or contiguous with respect to each other.

The person skilled in the art will recognize that a polypeptide with one or more arginine residues will be a polycationic peptide as long as the total positive electrical charge of the polypeptide at physiological pH is 2 or more, resulting not only from the positive electrical charges of the arginine residues but also from any other positively charged amino acids.

In an embodiment of the invention, the polycationic peptide of the invention is an arginine-rich sequence.

In a preferred embodiment of the invention, the arginine-rich sequence of the polycationic peptide of the invention is selected from the group consisting of SEQ ID NO: 1, SEQ ID NO: 2, SEQ ID NO: 3 and SEQ ID NO: 4.

(ii) Sequence which is Capable of Specifically Interacting with a Receptor on a Cell Surface and Promoting Internalization of the Fusion Protein on Said Cell

The terms “sequence which is capable of specifically interacting with a receptor on a cell surface and promoting internalization of the fusion protein on said cell”, as used herein, refers to any polypeptide sequence which binds to a receptor on the surface of a cell, wherein the receptor undergoes endocytosis in response to the binding of said polypeptide sequence. This binding specificity allows the delivery of the polypeptide sequence as well as the rest of the fusion protein which it is a part of to the cell, tissue or organ which expresses said receptor. In this way, a fusion protein comprising said polypeptide sequence will be directed specifically to said cells when administered to an animal or contacted in vitro with a population of cells of different types.

The term “receptor” denotes a cell-associated protein that binds to a bioactive molecule termed a “ligand”. Both “receptor” and “ligand” are commonly known to those skilled in the art.

As used herein, “internalization” refers to a process by which a molecule or a construct comprising a molecule binds to a target element on the outer surface of the cell membrane and the resulting complex is internalized by the cell. Internalization may be followed up by dissociation of the resulting complex within the cytoplasm. The target element, along with the molecule or the construct, may then localize to a specific cellular compartment. Preferably, the polycationic peptide of the invention, besides promoting internalization, will facilitate endosomal escape of the fusion protein.

In another preferred embodiment, the fusion protein of the invention comprises a peptide that allows the translocation of the protein to the cytosol and avoid its lysosomal degradation. In one embodiment, the peptide that allows the translocation of the protein to the cytosol is a peptide comprising or consisting of the KDEL sequence (SEQ DID NO. 48). In a further preferred embodiment the peptide that allows the translocation of the protein to the cytosol is located at the C-terminal domain of the fusion protein.

A wide array of uptake receptors and carriers, with an even wider number of receptor-specific ligands, are known in the art.

Non-limiting examples of receptors which may be targeted by the polycationic of the invention include an angiotensin receptor, a bombesin receptor, a bradykinin receptor, a calcitonin receptor, a chemokine receptor, a cholecystokinin receptor, a corticotropin-releasing factor receptor, an endothelin receptor, en ephrin receptor, a formylpeptide receptor, a Frizzled receptor, a galanin receptor, a the growth hormone secretagogue receptor (Ghrelin) receptor, a Kisspeptin receptor, a melanocortin receptor, Neuropeptide FF/neuropeptide AF receptor, a neuropeptide S receptor, a neuropeptide W/neuropeptide B receptor, a neuropeptide Y receptor, a neurotensin receptor, an orexin receptors, a peptide P518 receptor, a somatostatin receptor, a tachykinin receptor, a Toll-like receptor, a vasopressin and oxytocin receptor and a VEGF receptor.

In a preferred embodiment of the invention, the polycationic peptide comprising a sequence which is capable of specifically interacting with a receptor on a cell surface and promoting internalization of the fusion protein on said cell is a CXCR4 ligand.

The term “CXCR4”, as used herein, refers to a G protein-coupled, seven-transmembrane chemokine receptor. Like other chemokine receptors, CXCR4 plays an important role in immune and inflammatory responses by mediating the directional migration and activation of leukocytes CXCR4 is expressed or overexpressed in a variety of cancer cell lines and tissues including breast, prostate, lung, ovarian, colon, pancreatic, kidney, and brain, as well as non-Hodgkin's lymphoma and chronic lymphocytic leukemia. The only known ligand to CXCR4 is stromal cell-derived factor-1 (SDF-1, or CXCL12). The interaction between CXCR4 and SDF-1 plays an important role in multiple phases of tumorigenesis, including tumor growth, invasion, angiogenesis, and metastasis.

The expression “specifically binding to CXCR4”, as used herein refers to the ability of the conjugates of the invention to bind more frequently, more rapidly, with greater duration and/or with greater affinity to CXCR4 or cell expressing same than it does with alternative receptors or cells without substantially binding to other molecules.

Binding affinity is measured, for instance, as described by Tamamura et al. by the oil-cushion method [see Hesselgesset et al, 1998, J. Immunol., 160:877-883] comprising contacting the peptide with CXCR4-transfected cell line (e.g. CHO cells) and a labeled CXCR4 ligand (e.g. ¹²⁵I-SDF-1α) and measuring the inhibition percentage of the targeting peptide against the binding of the labeled CXCR4 ligand.

Specific binding can be exhibited, e.g., by a low affinity targeting agent having a Kd of at least about 10⁻⁴ M. e.g., if CXCR4 has more than one binding site for a ligand, a ligand having low affinity can be useful for targeting. Specific binding also can be exhibited by a high affinity ligands, e.g. a ligand having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater. Both low and high affinity-targeting ligands are useful for incorporation in the conjugates of the present invention.

The expression “facilitate endosomal escape”, as used herein, refers to the ability of the polycationic peptide or of the endosomal escape peptide to induce the release of the fusion proteins from the endosomal compartment after internalization by receptor-mediated endocytosis.

The ability of the conjugate of the invention to be internalized by cells expressing CXCR4 may be conveniently determined by fluorescence methods in the case that the conjugate comprises a fluorescent protein, such as GFP. Such fusion proteins can be obtained by preparing a recombinant nucleic acid wherein the nucleic acids encoding the T22 peptide and the fluorescent protein are fused in frame and expressed in an adequate host cell or organism. The fusion protein is then contacted with a culture of cells expressing CXCR4 or in vivo with a tissue which expresses CXCR4 for an appropriate amount of time, after which fluorescence microscopy may be used to determine whether the construct penetrated the cell. Presence of fluorescence in the cytoplasm may be further investigated by comparing the fluorescence microscopy image resulting from the fluorescent protein to that obtained with a known cytoplasmic stain.

In an even more preferred embodiment of the invention, the CXCR4 ligand is selected from the group comprising the T22 peptide (SEQ ID NO: 5), the V1 peptide (SEQ ID NO: 6), the CXCL12 peptide (SEQ ID NO: 7), the vCCL2 peptide (SEQ ID NO: 8) or a functionally equivalent variant thereof.

The T22 peptide corresponds to a peptide derived from the protein polyphemusin II (extracted from hemocyte debris from Lymulus polyphemus). The vCCL2 corresponds to the viral macrophage inflammatory protein-II, an homologue of human chemokine CCL2 encoded by human herpesvirus 8. The V1 peptide corresponds to residues 1-21 of the N-terminus of vCCL2. CXCL12, C-X-C motif chemokine 12, also known as stromal cell-derived factor 1 (SDF1), is a member of the chemokine family that acts as a pro-inflammatory mediator. All four peptides are known to have interactions with the CXCR4 receptor, as shown in Liang, X. 2008. Chem. Biol. Drug. Des. 72:91-110.

In one embodiment, the targeting peptide is the selected from the group consisting of:
the T140 peptide having the sequence RRX₁CYRKX₂PYRX₃CR (SEQ ID NO: 9) wherein X₁ is L-3-(2-naphtyl)alanine, X₂ is D-Lys and X₃ is L-Citrulline.
the TN14003 peptide having the sequence RRX₁CYX₂KX₃PYRX₄CR (SEQ ID NO: 10) wherein X₁ is L-3-(2-naphtyl)alanine, X₂ is L-Citrulline, X₃ is dLys and X₄ is L-Citrulline,
the TC14012 peptide having the sequence RRX₁CYEKX₂PYRX₃CR (SEQ ID NO: 11) wherein X₁ is L-3-(2-naphtyl)alanine, X₂ is D-Citrulline and X₃ is L-Citrulline,
the TE14011 peptide having the sequence RRX₁CYX₂KX₃PYRX₄CR (SEQ ID NO: 12) wherein X₁ is L-3-(2-naphtyl)alanine, X₂ is L-Citrulline, X₃ is D-Glu and X₄ is L-Citrulline and
the TZ14011 peptide having the sequence RRX₁CYX₂KX₃PYRX₄CR (SEQ ID NO: 13) wherein X₁ is L-3-(2-naphtyl)alanine, X₂ is L-Citrulline, X₃ is D-Lys and X₄ is L-Citrulline or the variant thereof wherein the N-terminal Arginine residue is acetylated (known Ac-TZ14011).

The terms “functional variant” and “functionally equivalent variant” are interchangeable and are herein understood as all those peptides derived from the T22, the VI, the CXCL12, and/or the vCCL2 peptides by means of modification, insertion and/or deletion of one or more amino acids, provided that the function of binding to CXCR4 and internalizing the fusion protein is substantially maintained.

In one embodiment, functionally equivalent variants of the cationic polypeptides are those showing a degree of identity with respect to the human T22, V1, CXCL12 and/or the vCCL2 peptides, according to their respective SEQ ID NOs, greater than at least 40%, at least 50%, at least 60%, at least 70%, at least 80%, at least 85%, at least 90%, at least 95%, at least 96%, at least 97%, at least 98% or at least 99%. The degree of identity between two amino acid sequences can be determined by conventional methods, for example, by means of standard sequence alignment algorithms known in the state of the art, such as, for example BLAST [Altschul S. F. et al., J. Mol. Biol., 1990 Oct. 5; 215(3):403-10]. The cationic polypeptides of the invention may include post-translational modifications, such as glycosylation, acetylation, isoprenylation, myristoylation, proteolytic processing, etc.

Alternatively, suitable functional variants of the cationic polypeptide are those wherein one or more positions contain an amino acid which is a conservative substitution of the amino acid present in the T22, V1, CXCL12, and/or vCCL2 peptides mentioned above. “Conservative amino acid substitutions” result from replacing one amino acid with another having similar structural and/or chemical properties For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). Selection of such conservative amino acid substitutions is within the skill of one of ordinary skill in the art and is described, for example by Dordo et al. et al., [J. Mol. Biol, 1999, 217; 721-739] and Taylor et al., [J. Theor. Biol., 1986, 119:205-218].

A suitable assay for determining whether a given peptide can be seen as a functionally equivalent variant thereof is, for instance, the following assay: a putative T22, V1, CXCL12 or vCCL2 peptide variant is fused in frame with a marker polypeptide (e.g. a fluorescent protein). Such fusion proteins can be obtained by preparing a recombinant nucleic acid wherein the nucleic acids encoding the peptide and the fluorescent protein are fused in frame and expressed in an adequate host cell or organism. The fusion protein is then contacted with a culture of cells CXCR4 (e.g. HeLa cells) for an appropriate amount of time after which fluorescence microscopy may be used to determine whether the construct penetrated the cell. If the peptide is a functionally equivalent variant of the corresponding peptide, the marker protein will be internalized and presence of fluorescence in the cytoplasm of the cell will be visible. Furthermore, the performance of the functionally equivalent variant can be assayed by comparing the fluorescence microscopy image resulting from the fluorescent protein to that obtained with a known cytoplasmic stain (e.g. DAPI).

(iii) GW-H1 Peptide

The GW-H1 peptide was previously described by Chen and colleagues [Chen, Y-L. S. et al. 2012. Peptides, 36:257-265]. The GW-H1 peptide was first selected as an antimicrobial peptide but it is also characterized by its capability to bind to cell membranes, internalize itself to the cytoplasm, and migrate to the nuclei in eukaryotic cells. Once inside the cell, GW-H1 is capable induce apoptosis. It has been proposed that GW-H1 exerts its cytolytic activity by folding into an amphipathic helix [Chen and colleagues, supra]. Therefore, this peptide is supposed to exert its cell lytic effects by two sequential events consisting on binding to cell membranes followed by permeabilization.

In a preferred embodiment of the invention, the polycationic peptide of the invention is the GW-H1 peptide, which has the SEQ ID NO: 14.

### (iv) CD44 Ligand

CD44 is a cell-surface transmembrane glycoprotein involved in cell-cell and cell-matrix interactions, cell adhesion and migration. CD44 has been implicated in inflammation and in diseases such as cancer [Bajorath, J. 2000. Proteins. 39:103-111]. Many isoforms are known, which are expressed in a cell-specific manner and also differentially glycosylated.

Accordingly, a “CD44 ligand” will be a molecule capable of binding to CD44. CD44 is the major surface receptor for Hyaluronan, a component of the extracellular matrix, but it has other ligands, such as chondroitin sulfate, the heparin-biding domain of fibronectin, osteopontin, serglycin, collagen and laminin. Besides, CD44 can also interact with metalloproteinases and selectins.

In an embodiment of the invention, the polycationic peptide of the invention is a CD44 ligand. In a preferred embodiment of the invention, the CD44 ligand is selected from the group consisting of A5G27 (SEQ ID NO: 15) and FNI/II/V (SEQ ID NO: 16).

The peptide FNI/II/V corresponds to the HBFN-fragment V of Fibronectin. The peptide A5G27 corresponds to a peptide of the α5 chain of Laminin [Pesarrodona, M. et al. 2014. Int. J. of Pharmaceutics. 473:286-295].

### (v) Peptide Capable of Crossing the Blood-Brain Barrier

It is well known in the art that one major obstacle for the development of therapeutic approaches for brain pathologies is the blood-brain barrier (BBB). The brain is shielded against potentially toxic substances by the presence of two barrier systems: the blood-brain barrier (BBB) and the blood-cerebrospinal fluid barrier (BCSFB). The BBB is considered to be the major route for the uptake of serum ligands since its surface area is approximately 5000-fold greater than that of BCSFB. The brain endothelium, which constitutes the BBB, represents the major obstacle for the use of potential drugs against many disorders of the CNS. As a general rule, only small lipophilic molecules may pass across the BBB, i.e., from circulating systemic blood to brain. Many drugs that have a smaller size or higher hydrophobicity show promising results in animal studies for treating CNS disorders.

Therefore, a “peptide capable of crossing the blood-brain barrier” will be a peptide capable of transporting itself as well as any molecule it is bound to, preferably a protein, from the blood torrent to the CNS.

In 1983 it was reported that a peptide, β-Casomorphin-5 could overcome the BBB [Ermisch, A. et al. 1983. J. of Neurochemistry. 41:1229-1233]. Since then, many other peptides with BBB-permeating properties have been identified, characterized and catalogued, and in 2012 a comprehensive database was established, as reported by Van Dorpe et al. [Van Dorpe, S. et al. 2012. Brain Struct. Funct. 217:687-718]. Most of the peptides listed in the aforementioned database are suitable for the fusion protein of the invention.

In an embodiment of the invention, the polycationic peptide of the invention is a peptide capable of crossing the blood-brain barrier. In a preferred embodiment of the invention, the peptide capable of crossing the blood-brain barrier is a selected from the group consisting of Seq-1-7 (SEQ ID NO: 17), Seq-1-8 (SEQ ID NO: 18), and Angiopep-2-7 (SEQ ID NO: 19).

### (vi) Cell Penetrating Peptide (CPP)

The terms “cell-penetrating peptide” (CPP) refers to a peptide, typically of about 5-60 amino acid residues in length, that can facilitate cellular uptake of molecular cargo, particularly proteins they are a part of. Proteins can present one or more CPPs. CPPs can also be characterized as being able to facilitate the movement or traversal of molecular cargo across/through one or more of a lipid bilayer, cell membrane, organelle membrane, vesicle membrane, or cell wall. A CPP herein will be polycationic. Examples of CPPs useful herein, and further description of CPPs in general, are disclosed in Schmidt et al. [2010. FEBS Lett. 584:1806-1813], Holm et al. [2006. Nature Protocols 1:1001-1005], Yandek et al, [2007. Biophys. J. 92:2434-2444], Morris et al. [2001. Nat. Biotechnol. 19:1173-1176]. and U.S. Patent Application Publication No. 2014/0068797. CPPs do not depend on transporters or receptors, facilitating the traffic of the proteins they are part of directly through the lipid bilayer without the need of participation by any other cell components.

(vii) Nucleolin-Binding Peptide

Nucleolin is an eukaryotic phosphoprotein that participates in ribosomal synthesis and maturation. This protein is present in multiple cellular locations. It has been described how cell-surface nucleolin is involved in signal transduction in cancerous cells [Reyes-Reyes, E. & Akiyama, S. K. 2008. Exp. Cell Res. 314:2212-2223] and also how the use of an antagonist of cell-surface nucleolin suppresses tumor growth and angiogenesis [Destouches, D. et al. 2008. PLoS One. 3(6):e2518].

Accordingly, a “nucleolin-binding peptide” is a peptide capable of binding to the nucleolin protein in a cell, preferably to the cell-surface expressed fraction of nucleolin.

In an embodiment of the invention, the polycationic peptide of the invention is a nucleolin-binding peptide.

The International Patent Application Publication with number WO 2011/031477 A2 offers numerous examples of nucleolin-binding peptides that would be suitable for use in the fusion protein of the invention.

In a preferred embodiment of the invention, the nucleolin-binding peptide of the invention is the peptide of sequence SEQ ID NO: 20.

### B. Positively Charged Amino Acid-Rich Region

The term “positively charged amino acid” or “second positively charged amino acid-rich region” as used herein, refers to a polypeptide sequence, different from the polycationic region or first positively charged amino acid-rich region characterized in that it contains multiple positively charged amino acids. In addition, the positively charged amino acid-rich region may be formed exclusively by positively charged amino acids or may contain other amino acids provided that the overall net charge of the region at pH 7 is positive. Thus, the positively charged amino acid-rich region sequence may comprise 33%, preferably 40%, preferably 45%, preferably 50%, preferably 55%, preferably 60%, preferably 65%, preferably 70%, preferably 75%, preferably 80%, preferably 85%, more preferably 90%, more preferably 95%, even more preferably 99%, yet even more preferably 100% of the amino acid residues of its complete sequence as positively charged amino acids residues.

The positively charged amino acid-rich region may contain only one type of positively charged amino acid or may contain more than one type of positively charged amino acid. In one embodiment, the positively charged amino acid-rich region is a polyhistidine region. In one embodiment, the positively charged amino acid-rich region is a polyarginine region. In one embodiment, the positively charged amino acid-rich region is a polyhistidine region. In one embodiment, the positively charged amino acid-rich region comprises lysine and arginines residues. In one embodiment, the positively charged amino acid-rich region comprises lysine and histidine residues. In one embodiment, the positively charged amino acid-rich region comprises arginine and histidine residues. In one embodiment, the positively charged amino acid-rich region comprises lysine, arginine and histidine residues

In some embodiments, the positively charged amino acid-rich region comprises at least 2, at least 3, at least 4, at least 5, at least 6, at least 7, at least 8, at least 9, at least 10, at least 11, at least 12, at least 13, at least 14, or at least 15 positively charged amino acids residues, wherein the positively charged amino acid can be histidine, lysine, arginine or combinations thereof.

In some embodiments, the positively charged amino acid-rich region comprises fewer than 100, fewer than 90, fewer than 80, fewer than 70, fewer than 60, fewer than 50, fewer than 40, fewer than 30, fewer than 29, fewer than 28, fewer than 27, fewer than 26, fewer than 25, fewer than 24, fewer than 23, fewer than 22, fewer than 21, fewer than 20, fewer than 19, fewer than 18, fewer than 17, fewer than 16, fewer than 15, fewer than 14, fewer than 13, fewer than 12, fewer than 11, fewer than 10 or less positively charged amino acids residues, wherein the positively charged amino acid can be histidine, lysine, arginine or combinations thereof.

In some embodiments, the positively charged amino acid-rich region comprises between 2 and 50 amino acids, between 2 and 40 amino acids, between 2 and 30 amino acids, between 2 and 25 amino acids, between 2 and 20 amino acids, between 2 and 10 amino acids or between 2 and 8 amino acids.

In some embodiments, the positively charged amino acid-rich region comprises between 3 and 50 amino acids, between 3 and 40 amino acids, between 3 and 30 amino acids, between 3 and 25 amino acids, between 3 and 20 amino acids, between 3 and 10 amino acids or between 3 and 8 amino acids. In some embodiments, the positively charged amino acid-rich region comprises between 4 and 50 amino acids, between 4 and 40 amino acids, between 4 and 30 amino acids, between 4 and 25 amino acids, between 4 and 20 amino acids, between 4 and 10 amino acids or between 4 and 8 amino acids. In some embodiments, the positively charged amino acid-rich region comprises between 5 and 50 amino acids, between 5 and 40 amino acids, between 5 and 30 amino acids, between 5 and 25 amino acids, between 5 and 20 amino acids, between 5 and 10 amino acids or between 5 and 8 amino acids.

In an embodiment of the invention, the positively charged amino acid-rich region of the fusion protein of the invention is a polyhistidine region. In a preferred embodiment of the invention, the polyhistidine region comprises between 2 and 10 contiguous histidine residues.

In an embodiment of the invention, the positively charged amino acid-rich region of the fusion protein of the invention is a polyarginine region. In a preferred embodiment of the invention, the polyarginine region comprises between 2 and 10 contiguous arginine residues.

In an embodiment of the invention, the positively charged amino acid-rich region of the fusion protein of the invention is a polylysine region. In a preferred embodiment of the invention, the polylysine region comprises between 2 and 10 contiguous polylysine residues.

### C. Relative Positions of the Elements of the Fusion Proteins and Linking Elements

The different elements of the fusion protein (polycationic peptide, intervening polypeptide region, and positively charged amino acid-rich region region) of the invention can be placed in any relative order provided that the polycationic peptide and the positively charged amino acid-rich region is functional on any position of the fusion protein and also the intervening polypeptide region remains functional totally or in part.

As used herein, the terms “N-terminal end”, “N-terminus”, and “amino-terminal end” of a polypeptide are indistinct. Equally, the terms “C-terminal end”, “C-terminus”, and “carboxi-terminal end” are considered equivalent. The terms are of common usage for the person skilled in the art regarding the free moieties of the amino acids at the ends of the polypeptide chains comprised by a protein.

Thus, in an embodiment of the invention, the polycationic peptide of the fusion protein is located at the N-terminal end of the protein, while the positively charged amino acid-rich region of the fusion protein is located at the C-terminal end of the protein. In another embodiment of the invention, the positively charged amino acid-rich region of the fusion protein is located at the N-terminal end of the protein, while the polycationic peptide of the fusion protein is located at the C-terminal end of the protein. In another embodiment of the invention, the intervening polypeptide region can be located at either the C-terminal end or the N-terminal end of the fusion protein, while the polycationic peptide is in the middle position of the fusion protein and the positively charged amino acid-rich region is at the end of the fusion protein opposite the Intervening polypeptide region, or the positively charged amino acid-rich region is in the middle position of the fusion protein and the polycationic peptide is located at the end of the fusion protein opposite the Intervening polypeptide region.

Accordingly, the relative order of the elements of the fusion protein according to the invention, can be:
N-Polycationic peptide-Intervening region polypeptide-positively charged amino acid-rich region-C;
N-positively charged amino acid-rich region-Intervening region polypeptide-Polycationic peptide-C;
N-Polycationic peptide-positively charged amino acid-rich region-Intervening region polypeptide-C;
N-positively charged amino acid rich region-Polycationic peptide-Intervening region polypeptide-C;
N-Intervening region polypeptide-Polycationic peptide-positively charged amino acid-rich region-C; or
N-Intervening region polypeptide-positively charged amino acid-rich region-Polycationic peptide-C

The terms “N-terminal end” and “C-terminal end” do not mean that the components need to be directly conjugated end-to-end, but that they maintain that relative order of positions regardless of the presence of additional elements at the end of either component or intercalated between them, such as linkers/spacers.

Therefore, the fusion protein of the invention comprises the aforementioned elements ((1) polycationic peptide, (2) intervening polypeptide region, and (3) positively charged amino acid-rich region) and these can be conjugated end-to-end but also may include one or more optional peptide or polypeptide “linkers” or “spacers” intercalated between them, linked, preferably by peptidic bond.

According to the invention, the spacer or linker amino acid sequences can act as a hinge region between components (1) and (2), (2) and (3), and (1) and (3) allowing them to move independently from one another while maintaining the three-dimensional form of the individual domains, such that the presence of peptide spacers or linkers does not alter the functionality of any of the components (1), (2) and (3). In this sense, a preferred intermediate amino acid sequence according to the invention would be a hinge region characterized by a structural ductility allowing this movement. In a particular embodiment, said intermediate amino acid sequence is a flexible linker. The effect of the linker region is to provide space between the components (1) and (2) and (2) and (3). It is thus assured that the secondary and tertiary structure of component (1), (2) or (3) is not affected by the presence of either of the others. The spacer is of a polypeptide nature. The linker peptide preferably comprises at least 2 amino acids, at least 3 amino acids, at least 5 amino acids, at least 10 amino acids, at least 15 amino acids, at least 20 amino acids, at least 30 amino acids, at least 40 amino acids, at least 50 amino acids, at least 60 amino acids, at least 70 amino acids, at least 80 amino acids, at least 90 amino acids or approximately 100 amino acids.

The spacer or linker can be bound to components flanking the two components of the conjugates of the invention by means of covalent bonds, preferably by peptide bonds; and also preferably the spacer is essentially afunctional, and/or is not prone to proteolytic cleavage, and/or does not comprise any cysteine residue. Similarly, the three-dimensional structure of the spacer is preferably linear or substantially linear.

The preferred examples of spacer or linker peptides include those that have been used to bind proteins without substantially deteriorating the function of the bound peptides or at least without substantially deteriorating the function of one of the bound peptides. More preferably the spacers or linkers used to bind peptides comprise coiled coil structures.

Preferred examples of linker peptides comprise 2 or more amino acids selected from the group consisting of glycine, serine, alanine and threonine. A preferred example of a flexible linker is a polyglycine linker. The possible examples of linker/spacer sequences include SGGTSGSTSGTGST (SEQ ID NO: 21), AGSSTGSSTGPGSTT (SEQ ID NO: 22) or GGSGGAP (SEQ ID NO: 23). These sequences have been used for binding designed coiled coils to other protein domains [Muller, K. M., Arndt, K. M. and Alber, T., Meth. Enzimology, 2000, 328: 261-281]. Further non-limiting examples of suitable linkers comprise the amino acid sequence GGGVEGGG (SEQ ID NO: 24), the sequence of 10 amino acid residues of the upper hinge region of murine IgG3 (PKPSTPPGSS, SEQ ID NO: 25), which has been used for the production of dimerized antibodies by means of a coiled coil [Pack, P. and Pluckthun, A., 1992, Biochemistry 31:1579-1584], the peptide of sequence APAETKAEPMT (SEQ ID NO: 26), the peptide of sequence GAP, the peptide of sequence AAA and the peptide of sequence AAALE.

Alternatively, the components of the fusion proteins of the invention can be connected by peptides the sequence of which contains a cleavage target site for a protease, thus allowing the separation of any of the components. Protease cleavage sites suitable for their incorporation into the polypeptides of the invention include enterokinase (cleavage site DDDDK, SEQ ID NO: 27), factor Xa (cleavage site IEDGR, SEQ ID NO: 28), thrombin (cleavage site LVPRGS, SEQ ID NO: 29), TEV protease (cleavage site ENLYFQG, SEQ ID NO: 30), PreScission protease (cleavage site LEVLFQGP, SEQ ID NO: 31), furin (cleavage site GNRVRRSV, SEQ ID NO. 46 or RHRQPRGWEQL, SEQ ID NO. 47), inteins and the like. In a preferred embodiment the target cleave site is for the protease furin (cleavage site GNRVRRSV, SEQ ID NO. 46 or RHRQPRGWEQL, SEQ ID NO. 47).

In another preferred embodiment, the cleavage target site for a protease is located between any of the components of the fusion protein of the invention. In a more preferred embodiment, the fusion protein comprises several cleavage target sites, each comprised between different components of the fusion protein, this is between the polycationinc peptide and the intervening peptide, and/or between the intervening peptide and the positively charged amino acid-rich region, more concretely, at the C-terminus of the polyactioinc peptide, at the N-terminus of the intervening peptide, at the C-terminus of the intervening peptide, and/or at the N-terminus of the positively charged amino acid-rich region. In an even more preferred embodiment the cleavage targe site is located at between the polycationic peptide and the intervening peptide, yet more preferably at the N-terminus of the intervening polypeptide. In another preferred embodiment it is located at the C-terminus of the polycationic peptide.

In another preferred embodiment the cleavage site of the invention is located at the C-terminus or N-terminus of a linking group as described herein, which is located between any of the components of the fusion proteins of the invention.

Thus, in an embodiment of the invention, the polycationic peptide is bound to the intervening polypeptide region through a linker. In another embodiment of the invention, the intervening polypeptide region is bound to the positively charged amino acid-rich region through a linker. In yet another embodiment of the invention, the polycationic peptide is bound to the intervening polypeptide region through a linker and the intervening polypeptide region is bound to the positively charged amino acid region through a linker also.

As the person skilled in the art will acknowledge, the linkers connecting the polycationic peptide to the intervening polypeptide region and the intervening polypeptide region to the positively charged amino acid-rich region may comprise the same sequence or different ones with the aforementioned limitation that the presence and/or sequence of the linkers does not result in functional alterations of the polycationic peptide, the intervening polypeptide region, and/or the positively charged amino acid-rich region (for instance, but not limited to, due to secondary or tertiary structure modifications of the fusion protein or formation of disulfide bonds).

The aforementioned considerations regarding the relative positions from the N-terminal end to the C-terminal end of the elements of the fusion protein apply also in the presence of linkers between them, independently of the number of them or what elements they are placed between. Therefore, the possible combinations and relative orders of elements will be the following (wherein the numbering stated above for the elements is retained: (1) polycationic peptide, (2) intervening polypeptide region, (3) positively charged amino acid-rich region):
N-(1)-(2)-(3)-C
N-(1)-linker-(2)-(3)-C
N-(1)-(2)-linker-(3)-C
N-(1)-linker-(2)-linker-(3)-C
N-(3)-(2)-(1)-C
N-(3)-linker-(2)-(1)-C
N-(3)-(2)-linker-(1)-C
N-(3)-linker-(2)-linker-(3)-C
N-(2)-(1)-(3)-C
N-(2)-linker-(1)-(3)-C
N-(2)-(1)-linker-(3)-C
N-(2)-linker-(1)-linker-(3)-C
N-(2)-(3)-(1)-C
N-(2)-linker-(3)-(1)-C
N-(2)-(3)-linker-(1)-C
N-(2)-linker-(3)-linker-(1)-C
N-(1)-(3)-(2)-C
N-(1)-(3)-linker-(2)-C
N-(1)-linker-(3)-(2)-C
N-(1)-linker-(3)-linker-(2)-C
N-(3)-(1)-(2)-C
N-(3)-linker-(1)-(2)-C
N-(3)-(1)-linker-(2)-C
N-(3)-linker-(1)-linker-(2)-C

In a preferred embodiment of the invention, the linkers of the fusion protein of the invention comprise the sequence GGSSRSS (SEQ ID NO: 32) sequence of the GGGNS sequence (SEQ ID NO: 33).

The aforementioned considerations regarding the relative positions from the N-terminal end to the C-terminal end of the elements of the fusion protein apply also in the presence of protease cleavage sites or a polypeptide containing a protease cleavage site between them, independently of the number of them or what elements they are placed between. Therefore, the possible combinations and relative orders of elements will be the following (wherein the numbering stated above for the elements is retained: (1) polycationic peptide, (2) intervening polypeptide region, (3) positively charged amino acid-rich region) and wherein the term “protease cleavage site” is to be understood as polypeptide region consisting of or comprising a protease cleavage site:
N-(1)-(2)-(3)-C
N-(1)-protease cleavage site-(2)-(3)-C
N-(1)-(2)-protease cleavage site-(3)-C
N-(1)-protease cleavage site-(2)-protease cleavage site-(3)-C
N-(3)-(2)-(1)-C
N-(3)-protease cleavage site-(2)-(1)-C
N-(3)-(2)-protease cleavage site-(1)-C
N-(3)-protease cleavage site-(2)-protease cleavage site-(1)-C
N-(2)-(1)-(3)-C
N-(2)-protease cleavage site-(1)-(3)-C
N-(2)-(1)-protease cleavage site-(3)-C
N-(2)-protease cleavage site-(1)-protease cleavage site-(3)-C
N-(2)-(3)-(1)-C
N-(2)-protease cleavage site-(3)-(1)-C
N-(2)-(3)-protease cleavage site-(1)-C
N-(2)-protease cleavage site-(3)-protease cleavage site-(1)-C
N-(1)-(3)-(2)-C
N-(1)-(3)-protease cleavage site-(2)-C
N-(1)-protease cleavage site-(3)-(2)-C
N-(1)-protease cleavage site-(3)-protease cleavage site-(2)-C
N-(3)-(1)-(2)-C
N-(3)-protease cleavage site-(1)-(2)-C
N-(3)-(1)-protease cleavage site-(2)-C
N-(3)-protease cleavage site-(1)-protease cleavage site-(2)-C.

In alternative embodiments, the fusion proteins according to the invention contain both linker regions connecting the elements of the fusion protein as well as protease cleavage sites between them, independently of the number of them or what elements they are placed between. Therefore, the possible combinations and relative orders of elements will be the following (wherein the numbering stated above for the elements is retained: (1) polycationic peptide, (2) intervening polypeptide region, (3) positively charged amino acid-rich region) and wherein the term “protease cleavage site” is to be understood as polypeptide region consisting of or comprising a protease cleavage site:
N-(1)-(2)-(3)-C
N-(1)-linker-protease cleavage site-(2)-(3)-C
N-(1)-protease cleavage site-linker-(2)-(3)-C
N-(1)-linker-protease cleavage site-linker-(2)-(3)-C
N-(1)-(2)-protease cleavage site-linker-(3)-C
N-(1)-(2)-linker-protease cleavage site-(3)-C
N-(1)-(2)-linker-protease cleavage site-linker-(3)-C
N-(1)-linker-protease cleavage site-(2)-protease cleavage site-(3)-C
N-(1)-protease cleavage site-linker (2)-protease cleavage site-(3)-C
N-(1)-linker-protease cleavage site-linker (2)-protease cleavage site-(3)-C
N-(1)-protease cleavage site-(2)-linker-protease cleavage site-(3)-C
N-(1)-protease cleavage site-(2)-protease cleavage site-linker-(3)-C
N-(1)-protease cleavage site-(2)-linker-protease cleavage site-linker-(3)-C
N-(1)-linker-protease cleavage site-(2)-linker-protease cleavage site-(3)-C
N-(1)-protease cleavage site-linker (2)-protease cleavage site-linker-(3)-C
N-(1)-linker-protease cleavage site-linker (2)-linker-protease cleavage site
linker-(3)-C
N-(3)-linker-protease cleavage site-(2)-(1)-C
N-(3)-protease cleavage site-linker-(2)-(1)-C
N-(3)-linker-protease cleavage site-linker-(2)-(1)-C
N-(3)-(2)-linker-protease cleavage site-(1)-C
N-(3)-(2)-protease cleavage site-linker-(1)-C
N-(3)-(2)-linker-protease cleavage site-linker (1)-C
N-(3)-linker-protease cleavage site-(2)-protease cleavage site-(1)-C
N-(3)-protease cleavage site-linker-(2)-protease cleavage site-(1)-C
N-(3)-linker-protease cleavage site-linker-(2)-protease cleavage site-(1)-C
N-(3)-protease cleavage site-(2)-linker-protease cleavage site-(1)-C
N-(3)-protease cleavage site-(2)-protease cleavage site-linker-(1)-C
N-(3)-protease cleavage site-(2)-linker-protease cleavage site-linker-(1)-C
N-(3)-linker-protease cleavage site-(2)-linker-protease cleavage site-(1)-C
N-(3)-protease cleavage site-linker-(2)-linker-protease cleavage site (1)-C
N-(3)-linker-protease cleavage site-linker-(2)-linker-protease cleavage site-(1)-C
N-(3)-linker-protease cleavage site-(2)-protease cleavage site-linker-(1)-C
N-(3)-protease cleavage site-linker-(2)-protease cleavage site-linker-(1)-C
N-(3)-linker-protease cleavage site-linker-(2)-protease cleavage site-linker(1)-C
N-(3)-linker-protease cleavage site-linker-(2)-linker-protease cleavage site
linker-(1)-C
N-(2)-linker-protease cleavage site-(1)-(3)-C
N-(2)-protease cleavage site-linker-(1)-(3)-C
N-(2)-linker-protease cleavage site-linker-(1)-(3)-C
N-(2)-(1)-linker-protease cleavage site-(3)-C
N-(2)-(1)-protease cleavage site-linker-(3)-C
N-(2)-(1)-linker-protease cleavage site-linker-(3)-C
N-(2)-linker-protease cleavage site-(1)-protease cleavage site-(3)-C
N-(2)-protease cleavage site-linker-(1)-protease cleavage site-(3)-C
N-(2)-linker-protease cleavage site-linker-(1)-protease cleavage site-(3)-C
N-(2)-protease cleavage site-(1)-linker-protease cleavage site-(3)-C
N-(2)-protease cleavage site-(1)-protease cleavage site-linker-(3)-C
N-(2)-protease cleavage site-(1)-linker-protease cleavage site-linker-(3)-C
N-(2)-linker-protease cleavage site-(3)-(1)-C
N-(2)-protease cleavage site-linker-(3)-(1)-C
N-(2)-linker-protease cleavage site-linker-(3)-(1)-C
N-(2)-(3)-linker-protease cleavage site-(1)-C
N-(2)-(3)-protease cleavage site-linker-(1)-C
N-(2)-(3)-linker-protease cleavage site-linker-(1)-C
N-(2)-linker-protease cleavage site-(3)-protease cleavage site-(1)-C
N-(2)-protease cleavage site-linker-(3)-protease cleavage site-(1)-C
N-(2)-linker-protease cleavage site-linker-(3)-protease cleavage site-(1)-C
N-(2)-protease cleavage site-(3)-linker-protease cleavage site-(1)-C
N-(2)-protease cleavage site-(3)-protease cleavage site-linker-(1)-C
N-(2)-protease cleavage site-(3)-linker-protease cleavage site-linker-(1)-C
N-(1)-(3)-linker-protease cleavage site-(2)-C
N-(1)-(3)-protease cleavage site-linker-(2)-C
N-(1)-(3)-linker-protease cleavage site-linker-(2)-C
N-(1)-linker-protease cleavage site-(3)-(2)-C
N-(1)-protease cleavage site-linker-(3)-(2)-C
N-(1)-linker-protease cleavage site-linker-(3)-(2)-C
N-(1)-linker-protease cleavage site-(3)-protease cleavage site-(2)-C
N-(1)-protease cleavage site-linker-(3)-protease cleavage site-(2)-C
N-(1)-linker-protease cleavage site-linker-(3)-protease cleavage site-(2)-C
N-(1)-protease cleavage site-(3)-linker-protease cleavage site-(2)-C
N-(1)-protease cleavage site-(3)-protease cleavage site-linker-(2)-C
N-(1)-protease cleavage site-(3)-linker-protease cleavage site-linker-(2)-C
N-(3)-linker-protease cleavage site-(1)-(2)-C
N-(3)-protease cleavage site-linker-(1)-(2)-C
N-(3)-linker-protease cleavage site-linker-(1)-(2)-C
N-(3)-(1)-linker-protease cleavage site-(2)-C
N-(3)-(1)-protease cleavage site-linker-(2)-C
N-(3)-(1)-linker-protease cleavage site-linker-(2)-C
N-(3)-linker-protease cleavage site-(1)-protease cleavage site-(2)-C.
N-(3)-protease cleavage site-linker-(1)-protease cleavage site-(2)-C.
N-(3)-linker-protease cleavage site-linker-(1)-protease cleavage site-(2)-C.
N-(3)-linker-protease cleavage site-(1)-linker-protease cleavage site-(2)-C.
N-(3)-protease cleavage site-linker-(1)-linker-protease cleavage site-(2)-C.
N-(3)-linker-protease cleavage site-linker-(1)-linker-protease cleavage site-(2)-C.
N-(3)-linker-protease cleavage site-(1)-protease cleavage site-linker-(2)-C.
N-(3)-protease cleavage site-linker-(1)-protease cleavage site-linker-(2)-C.
N-(3)-linker-protease cleavage site-linker-(1)-protease cleavage site-linker-(2)-C.
N-(3)-linker-protease cleavage site-(1)-linker-protease cleavage site-linker
(2)-C.
N-(3)-protease cleavage site-linker-(1)-linker-protease cleavage site-linker-(2)-C.
N-(3)-linker-protease cleavage site-linker-(1)-linker-protease cleavage site-linker-(2)-C.
N-(3)-protease cleavage site-(1)-linker-protease cleavage site-(2)-C.
N-(3)-protease cleavage site-(1)-protease cleavage site-linker-(2)-C.
N-(3)-protease cleavage site-(1)-linker-protease cleavage site-linker (2)-C.

### D. Intervening Polypeptide Region

The terms “intervening polypeptide region” and “intervening region” are herein considered equivalent.

The intervening polypeptide region of the fusion proteins of the invention comprises a physiologically functional peptide, meaning that its interaction with the cellular components results in physiological changes. Accordingly, linker regions connecting the different elements of the fusion protein according to the invention are not considered intervening regions. Thus, in preferred embodiments, the intervening region comprises at least 5, at least 10, at least 15, at least 20, at least 25, at least 30, at least 35, at least 40, at least 50, at least 60, at least 70, at least 80, at least 90, at least 100 or more amino acids.

In an embodiment of the invention, the intervening polypeptide region of the fusion proteins of the invention is a therapeutic agent.

The term “therapeutic” is used in a generic sense and includes treating agents, prophylactic agents, and replacement agents.

The nature of the intervening region is polypeptidic, as it is part of the fusion protein of the invention with the polycationic peptide and the positively charged amino acid-rich region.

Suitable polypeptides that can be used as components of the intervening region include any polypeptide which is capable of promoting a decrease in cell proliferation rates.

Examples of therapeutic proteins suitable for use in the intervening region of the fusion proteins of the invention include, but are not limited to, a cytotoxic polypeptide, an antiangiogenic polypeptide, a polypeptide encoded by a tumor suppressor gene, a polypeptide encoded by a polynucleotide which is capable of activating the immune response towards a tumor.

Thus, in an embodiment of the invention, the therapeutic agent of the intervening region of the fusion protein of the invention is selected from the group consisting of
(i) a cytotoxic polypeptide,
(ii) an antiangiogenic polypeptide,
(iii) a polypeptide encoded by a tumor suppressor gene,
(iv) a pro-apoptotic polypeptide,
(v) a polypeptide having anti-metastatic activity,
(vi) a polypeptide encoded by a polynucleotide which is capable of activating the immune response towards a tumor,
(vii) a chemotherapy agent,
(viii) an antiangiogenic molecule,
(ix) a polypeptide encoded by a suicide gene,
(x) a chaperone or an inhibitor of protein aggregation.

### (i) Cytotoxic Polypeptides

As used herein, the term cytotoxic polypeptide refers to an agent that is capable of inhibiting cell function. The agent may inhibit proliferation or may be toxic to cells. Any polypeptides that when internalized by a cell interfere with or detrimentally alter cellular metabolism or in any manner inhibit cell growth or proliferation are included within the ambit of this term, including, but not limited to, agents whose toxic effects are mediated when transported into the cell and also those whose toxic effects are mediated at the cell surface. Useful cytotoxic polypeptides include proteinaceous toxins and bacterial toxins.

Examples of proteinaceous cell toxins useful for incorporation into the conjugates according to the invention include, but are not limited to, type one and type two ribosome inactivating proteins (RIP). Useful type one plant RIPs include, but are not limited to, dianthin 30, dianthin 32, lychnin, saporins 1-9, pokeweed activated protein (PAP), PAP II, PAP-R, PAP-S, PAP-C, mapalmin, dodecandrin, bryodin-L, bryodin, Colicin 1 and 2, luffin-A, luffin-B, luffin-S, 19K-protein synthesis inhibitory protein (PSI), 15K-PSI, 9K-PSI, alpha-kirilowin, beta-kirilowin, gelonin, momordin, momordin-II, momordin-Ic, MAP-30, alpha-momorcharin, beta-momorcharin, trichosanthin, TAP-29, trichokirin; barley RIP; flax RIP, tritin, corn RIP, Asparin 1 and 2 [Stirpe et al., 1992. Bio/Technology 10:405-12]. Useful type two RIPs include, but are not limited to, volkensin, ricin, nigrin-b, CIP-29, abrin, modeccin, ebulitin-[alpha], ebulitin-[beta], ebultin-[gamma], vircumin, porrectin, as well as the biologically active enzymatic subunits thereof [Stirpe et al., 1992. Bio/Technology 10:405-12; Pastan et al., 1992. Annu. Rev. Biochem. 61:331-54; Brinkmann and Pastan, 1994. Biochim. et Biophys. Acta 1198:27-45,; and Sandvig and Van Deurs, 1996. Physiol. Rev. 76:949-66].

Examples of bacterial toxins useful as cell toxins include, but are not limited to, shiga toxin and shiga-like toxins (i.e., toxins that have the same activity or structure), as well as the catalytic subunits and biologically functional fragments thereof. Additional examples of useful bacterial toxins include, but are not limited to, Pseudomonas exotoxin and Diphtheria toxin [Pastan et al., 1992. Annu. Rev. Biochem. 61:331-54; and Brinkmann and Pastan, 1994. Biochim. et Biophys. Acta 1198:27-45]. Truncated forms and mutants of the toxin enzymatic subunits also can be used as a cell toxin moiety. Other targeted agents include, but are not limited to the more than 34 described Colicin family of RNase toxins which include colicins A, B, D, E1-9, cloacin DF13 and the fungal RNase, [alpha]-sarcin [Ogawa et al. 1999. Science 283: 2097-100; Smarda et al., 1998. Folia Microbiol (Praha) 43:563-82; Wool et al., 1992. Trends Biochem. Sci., 17: 266-69].

### (ii) Antiangiogenic Polypeptides

Proliferation of tumor cells relies heavily on extensive tumor vascularization, which accompanies cancer progression. Thus, inhibition of new blood vessel formation with anti-angiogenic agents and targeted destruction of existing blood vessels have been introduced as effective and relatively non-toxic approaches to tumor treatment.

The term “anti-angiogenic polypeptide”, as used herein, denotes a polypeptide capable of inhibiting angiogenesis. Suitable antiangiogenic polypeptides include, without limitation, angiostatin, endostatin, anti-angiogenic anti-thrombin III, sFRP-4 as described in WO2007115376, and an anti-VEGF antibody such as anibizumab, bevacizumab (avastin), Fab IMC 1121 and F200 Fab.

(iii) Polypeptides Encoded by a Tumor Suppressor Gene

As used herein, a “tumor suppressor” is a gene or gene product that has a normal biological role of restraining unregulated growth of a cell. The functional counterpart to a tumor suppressor is an oncogene-genes that promote normal cell growth may be known as “proto-oncogenes” A mutation that activates such a gene or gene product further converts it to an “oncogene”, which continues the cell growth activity, but in a dysregulated manner Examples of tumor suppressor genes and gene products are well known in the literature and may include PTC, BRCA1, BRCA2, p16, APC, RB, WTI, EXT1, p53, NF1, TSC2, NF2, VHL, ST7, ST14, PTEN, APC, CD95 or SPARC.

### (iv) Pro-Apoptotic Polypeptides

The term “pro-apoptotic polypeptides”, as used herein, refers to a protein which is capable of inducing cell death in a cell or cell population. The overexpression of these proteins involved in apoptosis displaces the careful balance between anti-apoptotic and pro-apoptotic factors towards an apoptotic outcome. Suitable pro-apoptotic polypeptides include, without limitation, pro-apoptotic members of the BCL-2 family of proteins such as BAX, BAK, BOK/MTD, BID, BAD, BIK/NBK, BLK, HRK, BIM/BOD, BNIP3, NIX, NOXA, PUMA, BMF, EGL-I, and viral homologs, caspases such as caspase-8, the adenovirus E4orf4 gene, p53 pathway genes, pro-apoptotic ligands such as TNF, FasL, TRAIL and/or their receptors, such as TNFR, Fas, TRAIL-R1 and TRAIL-R2.

(v) Polypeptides with Anti-Metastatic Activity

The term “metastasis suppressor” as used herein, refers to a protein that acts to slow or prevent metastases (secondary tumors) from spreading in the body of an organism with cancer. Suitable metastasis suppressor include, without limitation, proteins such as BRMS 1, CRSP3, DRG1, KAI1, KISS-1, NM23, a TIMP-family protein and uteroglobin.

### (vi) Polypeptides Encoded by a Polynucleotide Capable of Activating the Immune Response Towards a Tumor

As used herein, an immunostimulatory polypeptide agent is a polypeptide encoded by a polynucleotide which is capable of activating or stimulating the immune response (including enhancing a pre-existing immune response) in a subject to whom it is administered, whether alone or in combination with another agent. Suitable non-limiting examples of immunostimulatory peptides include flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

(vii) Chemotherapy Agents

It will be understood that the term “chemotherapeutic agents” refers to anti-cancer agents.

As used herein, an anti-cancer agent is an agent that at least partially inhibits the development or progression of a cancer, including inhibiting in whole or in part symptoms associated with the cancer even if only for the short term.

Suitable anti-cancer agents include Interferon alpha-2a; Interferon alpha-2b; Interferon alpha-n1; Interferon alpha-n3; Interferon beta-I a; Interferon gamma-I b.

The anti-cancer agent may be an enzyme inhibitor including without limitation tyrosine kinase inhibitor, a CDK inhibitor, a MAP kinase inhibitor, or an EGFR inhibitor. The CDK inhibitor may be without limitation p21, p27, p57, p15, p16, p18, or p19.

The anti-cancer agent may be an antibody or an antibody fragment including without limitation an antibody or an antibody fragment including but not limited to bevacizumab (AVASTIN), trastuzumab (HERCEPTIN), alemtuzumab (CAMPATH, indicated for B cell chronic lymphocytic leukemia,), gemtuzumab (MYLOTARG, hP67.6, anti-CD33, indicated for leukemia such as acute myeloid leukemia), rituximab (RITUXAN), tositumomab (BEXXAR, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX, indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (anti-TAG-72), I0R-C5, 10R-T6 (anti-CD 1), IOR EGF/R3, celogovab (ONCOSCINT OV 103), epratuzumab (LYMPHOCIDE), pemtumomab (THERAGYN), and Gliomab-H (indicated for brain cancer, melanoma).

(viii) Antiangiogenic Molecules

It is also contemplated that in certain embodiments the intervening region of the fusion protein of the invention corresponds to a protein that acts as an angiogenesis inhibitor is targeted to a tumor. These agents include, in addition to the anti-angiogenic polypeptides mentioned above, Marimastat; AG3340; COL-3, BMS-275291, Thalidomide, Endostatin, SU5416, SU6668, EMD121974, 2-methoxyoestradiol, carboxiamidotriazole, CMlOl, pentosan polysulphate, angiopoietin 2 (Regeneron), herbimycin A, PNU145156E, 16K prolactin fragment, Linomide, thalidomide, pentoxifylline, genistein, TNP470, endostatin, paclitaxel, accutin, angiostatin, cidofovir, vincristine, bleomycin, AGM-1470, platelet factor 4 or minocycline. Also included are VEGF inhibitors including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin.

### (ix) Polypeptide Encoded by a Suicide Gene

In the context of the invention, a “polypeptide encoded by a suicide gene” refers to a polypeptide the expression of which results in cell expressing it killing itself through apoptosis. This approach comprises the selective expression of the suicide gene only in particular cells, though the use of specific promoters, for instance, that would activate only in cells actually suffering the disease to be suppressed.

This approach comprises the use of pairs of enzyme and pro-drug, in which the enzyme is used to transform the target cells previously to the administration of the pro-drug, which under the action of the enzyme becomes a product toxic for the cell that kickstarts the apoptotic process. Usually, the enzymes of these systems of suicide gene therapy are usually not found in the same organism in which they are intended to be expressed, and so in mammals have been used enzymes obtained from bacteria, fungi or other organisms. This strategy has several known examples [reviewed in Karjoo, Z. et al. 2016. Adv. Drug Deliv. Rev. 99 (Pt. A):123-128], such as the thymidine kinase/ganciclovir system, the cytosine deaminase/5-fluorocytosine system, the nitroreductase/CB1954 system, carboxypeptidase G2/Nitrogen mustard system, cytochrome P450/oxazaphosphorine system, purine nucleoside phosphorylase/6-methylpurine deoxyriboside (PNP/MEP), the horseradish peroxidase/indole-3-acetic acid system (HRP/IAA), and the carboxylesterase/irinotecan (CE/irinotecan) system, the truncated EGFR, inducible caspase (“iCasp”), the the E. coli gpt gene, the E. coli Deo gene and nitroreductase.

### (x) Chaperone and Inhibitors of Protein Aggregation

As used herein, “chaperone polypeptide” or “chaperon” refers to a protein molecule that assists in folding or unfolding of protein molecules and/or assembly or disassembly of macromolecular structures. Exemplary chaperones include, but are not limited to, ABCE1 ATP-binding cassette sub-family E member 1; AHSA1 Activator of 90 kDa heat shock protein ATPase homolog 1; ANP32B acidic leucine-rich nuclear phosphoprotein 32 family; BAG6 Large proline-rich protein BAG6; BCS1L mitochondrial chaperone BCS1; CALR calreticulin; CANX calnexin; CCT2 T-complex protein 1 subunit beta CCT3 T-complex protein 1 subunit gamma CCT4 T-complex protein 1 subunit delta CCT5 T-complex protein 1 subunit epsilon CCT6A T-complex protein 1 subunit zbeta CCT7 T-complex protein 1 subunit beta CD74 H-2 class II histocompatibility antigen gamma chai; CDC37 Hsp90 co-chaperone Cdc37; CLGN calmegin; DNAJA1 DnaJ homolog subfamily A member 1; DNAJC1 DnaJ homolog subfamily C member 1; DNAJC11 DnaJ homolog subfamily C member 11; HSP90AA1 Heat shock protein HSP 90-alpha HSP90AB1 Heat shock protein HSP 90-beta HSP90B1 Endoplasmin; HSPA1B Heat shock 70 kDa protein 1A/1B; HSPA2 Heat shock-related 70 kDa protein 2; HSPA8 Heat shock cognate 71 kDa protein; HSPA9 Stress-70 protein, mitochondrial; HSPD1 60 kDa heat shock protein, mitochondrial; HYOU1 Hypoxia up-regulated protein 1; NDUFAF2 Mimitin, mitochondrial; SCO1 Protein SCO1 homolog, mitochondrial; SCO2 Protein SCO2 homolog, mitochondrial; ST13 Hsc70-interacting protein; TBCD Tubulin-specific chaperone D; TCP1 T-complex protein 1 subunit alpha TIMMDC1 Translocase of inner mitochondrial membrane domain; and TMEM126B Transmembrane protein 126B.

Thus, in an embodiment of the invention, the therapeutic agent of the intervening region of the fusion protein of the invention is a cytotoxic polypeptide.

In a preferred embodiment of the invention, the cytotoxic polypeptide of the intervening region of the fusion protein is selected from the group consisting of the BH3 domain of BAK, PUMA GW-H1, the Diphtheria toxin, the Pseudomonas exotoxin and Ricin. In a further preferred embodiment of the invention, the cytotoxic polypeptide of the intervening region of the fusion protein is a truncated form or a mutant of the peptide selected from the group indicated just before, preferably from the group consisting of the Diphtheria toxin, the Pseudomonas exotoxin and Ricin.

As used herein “BAK” refers to the well-known pro-apoptotic factor belonging to the Bcl-2 protein family that triggers programmed cell death by caspase-dependent apoptotic pathway through inactivating anti-apoptotic proteins, permeabilizing the mitochondrial membrane, and consequently, releasing cytochrome C and other mitochondrial cell death factors. [as seen in Llambi, F. et al. 2011. Mol. Cell, 44:517-31]. In one embodiment, BAK refers to full length BAK (SEQ ID NO: 34). In other embodiment, BAK refers to any truncated form thereof containing the functional BH3 domain (SEQ ID NO: 35). The experiments provided in the present invention show that BH3 BAK was still functional as assembled into cell-targeted nanoparticles.

As used herein, “PUMA” refers to a protein characterized by a full sequence corresponding to SEQ ID NO: 36) which is a (Bcl-2 homology 3) BH3-only protein that triggers cell death by interacting with pro and antiapoptotic proteins of the Bcl-2 family.

As used herein, GW-H1 refers to a polypeptide having the sequence of SEQ ID NO: 14 which exerts its cytolytic activity by folding into an amphipathic helix. As shown in the examples of the present invention, GW-H1 shows a milder effect than the other tested constructs but in this form the nanomaterial is supposed to exert cell lytic effects by two sequential events consisting on binding to cell membranes followed by permeabilization.

As used herein “the Diphtheria toxin” refers to the exotoxin of the Corynebacterium diphtheriae, and “the Pseudomonas exotoxin” refers to the exotoxin A of the Pseudomonas aeruginosa which belongs to the family of ADP-ribosylating toxins. Both toxins are proteins that act on eukaryotic Elongation Factor-2 (eEF-2), basically inhibiting the translational activity of the cell that incorporates them and inducing apoptosis. The structure of both toxins presents a receptor-binding domain (that binds to a surface receptor of the cell and induces endocytosis; heparin binding epidermal growth factor precursor in the case of diphtheria toxin, CD91 in the case of the exotoxin A), a translocation domain, and a catalytic domain, herein also referred to as “active segment”, that performs the action on eEF-2. The catalytic domain or active segment of the diphtheria toxin corresponds to SEQ ID NO: 37, while the catalytic domain or active segment of the exotoxin A of P. aeruginosa corresponds to SEQ ID NO: 38 [an overview is provided in Shapira, A. & Benhar, I., 2010, Toxins, 2:2519-2583].

In a preferred embodiment, embodiment the diphtheria toxin of the invention is a truncated or mutant form of the exotoxin of the Corynebacterium diphtheriae. In a further preferred embodiment, the diphtheria toxin of the invention contains the translocation and catalytic domains of the diphtheria toxin. Said diphtheria toxin is referred herein as DITOX and has the sequence of SEQ ID NO.43.

In another preferred embodiment the Pseudomonas exotoxin of the invention is a truncated or mutant form of the exotoxin A of the Pseudomonas aeruginosa. In a further preferred embodiment the Pseudomonas exotoxin of the invention is based on the de-immunized catalytic domain of Pseudomonas aeruginosa exotoxin A in which point mutations that disrupt B and T cell epitopes have been incorporated. Said Pseudomonas exotoxin is referred herein as PE24 and has the sequence of SEQ ID NO. 44.

As used herein “Ricin” refers to the ribosome inactivating protein (RIP) originally extracted from the seeds of Ricinus communis of approximately 65 KDa which consists of two chains linked by a disulfide bond: the chain A with N-glycosidase enzymatic activity and the chain B with lectin properties which binds carbohydrate ligands on target cell surface. In a preferred embodiment the Ricin of the invention is a truncated or mutant form of the Ricin extracted from the seeds of Ricinus communis. In a further preferred embodiment the Ricin of the invention is a mutated version of the ricin A chain. In an even more preferred embodiment, said mutated ricin A chain consists on a ricin A chain with the mutation N132A, to suppress the vascular leak syndrome while maintaining the cytotoxic activity when administered. Said mutated Ricin A chain is referred herein as mRTA and has the sequence of SEQ ID NO. 45. In a preferred embodiment, the Ricin of the invention consists on the mRTA.

In a preferred embodiment, the intervening polypeptide is a bacterial toxin, the polycationic peptide is T22 and the positively charged amino acid-rich region is a polyhistidine, and, more particularly, an hexahistidine, wherein the T22 peptide and the bacterial toxin are connected by a linker having the sequence GGSSRSS and a furin cleavage site having the sequence GNRVRRSV. In a preferred embodiment, the bacterial toxin is a modified Diphtheria toxin comprising the A-fragment and the T-domain of the B-fragment but lacking the R-domain of the B-fragment. In a more preferred embodiment the bacterial toxin is the modified Diphtheria toxin corresponding to SEQ ID NO. 37, even more preferably the bacterial toxin is the modified Diphtheria toxin DITOX corresponding to SEQ ID NO. 43. In another embodiment, the bacteria toxin is the Pseudomonas exotoxin. In a more preferred embodiment the bacteria toxin is the Pseudomonas exotoxin with SEQ ID NO. 38, even more preferably the bacteria toxin is the Pseudomonas exotoxin PE24 with SEQ ID NO. 44.

In a preferred embodiment, the intervening polypeptide is a bacterial toxin, the polycationic peptide is T22 and the positively charged amino acid-rich region is a polyhistidine, and, more particularly, an hexahistidine wherein the T22 peptide and the bacterial toxin are connected by a linker having the sequence GGSSRSS, a furin cleavage site having the sequence RHRQPRGWEQL and a second linker having the GGS sequence and further comprising a KDEL sequence at the C-terminus after the positively charged amino acid-rich region. In a preferred embodiment, the bacterial toxin is a modified Diphtheria toxin comprising the A-fragment and the T-domain of the B-fragment but lacking the R-domain of the B-fragment. In a more preferred embodiment the bacterial toxin is the modified Diphtheria toxin corresponding to SEQ ID NO. 37. In a yet more preferred embodiment the bacterial toxin is the modified Diphtheria toxin DITOX corresponding to SEQ ID NO. 43. In another embodiment, the bacteria toxin is the Pseudomonas exotoxin. In a more preferred embodiment the bacteria toxin is the Pseudomonas exotoxin with SEQ ID NO. 38, even more preferably the Pseudomonas exotoxin PE24 with SEQ ID NO. 44.

In a preferred embodiment, the intervening polypeptide is ricin, the polycationic peptide is T22 and the positively charged amino acid-rich region is a polyhistidine and, more particularly, an hexahistidine and further comprising a KDEL sequence at the C-terminus after the positively charged amino acid-rich region. In a preferred embodiment, the fusion protein further comprises a linker region at the C-terminus of the T22 peptide comprising the sequence GGSSRSS. In another embodiment, the fusion protein further comprises a cleavage site for furin having the sequence RHRQPRGWEQL which connects the C-terminus of the linker region and a second linker region having the sequence GGS. In a preferred embodiment, the intervening polypeptide is a modified ricin carrying a N132A mutation aimed at suppressing the vascular leak syndrome. In another preferred embodiment, the intervening polypeptide is the ricin A chain. In another embodiment, the intervening polypeptide is the ricin A chain carrying a N132A mutation.

In the fusion protein of the invention, the intervening polypeptide region is not a fluorescent protein or p53.

In a preferred embodiment, the intervening polypeptide is not a fluorescent protein. It will be understood that the fusion protein of the invention may still comprise one or more fluorescent proteins within its structure provided that the fluorescent protein is not the intervening polypeptide. Accordingly, in one embodiment, if the fusion protein according to the invention contains a single intervening polypeptide, then this polypeptide is not a fluorescent protein. In another embodiment, if the fusion protein of the invention contains one or more additional polypeptides in addition to the intervening polypeptide, then the additional polypeptide or polypeptides may be a fluorescent protein. The term “intervening polypeptide” does not include any linker region forming part of fusion protein and connecting the different elements of the fusion protein. The fluorescent protein is selected from the group consisting of green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. In other embodiments, the intervening polypeptide is not a fluorescent protein selected from the group consisting of the mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like.

In preferred embodiments, the intervening polypeptide is not p53 or a p53 isoform encoded by the TP53 gene such as p53a, p53p, p53y, A40p53a, A40p53p, A40p53y, A133p53a, A133p53p, A133p53y, A160p53a, A160p53p, A160p53y and the like.

### E. Reporter Proteins

In another embodiment of the invention, the fusion protein of the invention further comprises a reporter protein. It will be understood that the reporter protein, as used herein, is different from the intervening polypeptide.

The person skilled in the art will acknowledge the term “reporter protein” as referring to a protein resulting from the expression of a “reporter gene”. Reporter proteins are well known and commonly used in the art as markers suitable for multiple purposes, such as location of the expression of the reporter genes in tissues, cells or subcellular locations, protein-protein interactions, transport across the plasmatic membranes or endomembranes, vesicular traffic, ligand-receptor interactions, etcetera.

Useful reporter proteins in the context of the present invention include luciferase-4-monooxygenase from Photinus pyralis, β-galactosidase, thymidine kinase, and the like. Preferred reporter proteins suitable for the fusion protein of the invention are also fluorescent proteins, such as the green fluorescent protein (GFP, first discovered in Aequorea victoria), the red fluorescent protein (REP), the yellow fluorescent protein (YFP), the blue fluorescent protein (BFP) or any other variant, examples of which can be found in Kremers et al. [Kremers, G-J- et al. 2011. J. Cell Sci. 124:157-160].

Thus, in a preferred embodiment of the invention, the reporter protein of the fusion protein of the invention is a fluorescent protein.

The fluorescent protein comprised by the fusion protein of the invention is directly adjacent to the positively charged amino acid-rich region or separated by a linker. The relative position of the positively charged amino acid-rich region, however, remains as per the aforementioned considerations about the relative position of the elements of the fusion protein. Hence, independently of the position of the fusion protein, the fluorescent protein is always adjacent to it, either directly or separated by a linker.

Accordingly, in the embodiments of the invention comprising a fluorescent protein the possible relative positions of the elements of the fusion protein of the invention would fit the following scheme (wherein FP refers to a fluorescent protein and the numbering stated above for the elements is retained: (1) polycationic peptide, (2) intervening polypeptide region, (3) positively charged amino acid-rich region):
N-(1)-(2)-FP-(3)-C
N-(1)-linker-(2)-FP-(3)-C
N-(1)-(2)-linker-FP-(3)-C
N-(1)-linker-(2)-linker-FP-(3)-C
N-(3)-FP-(2)-(1)-C
N-(3)-FP-linker-(2)-(1)-C
N-(3)-FP-(2)-linker-(1)-C
N-(3)-FP-linker-(2)-linker-(3)-C
N-(1)-(2)-FP-linker-(3)-C
N-(1)-linker-(2)-FP-linker-(3)-C
N-(1)-(2)-linker-FP-linker-(3)-C
N-(1)-linker-(2)-linker-FP-linker-(3)-C
N-(3)-linker-FP-(2)-(1)-C
N-(3)-linker-FP-linker-(2)-(1)-C
N-(3)-linker-FP-(2)-linker-(1)-C
N-(3)-linker-FP-linker-(2)-linker-(3)-C
N-(2)-(1)-FP-(3)-C
N-(2)-linker-(1)-FP-(3)-C
N-(2)-(1)-linker-FP-(3)-C
N-(2)-linker-(1)-linker-FP-(3)-C
N-(2)-FP-(3)-(1)-C
N-(2)-(3)-FP-(1)-C
N-(2)-linker-FP-(3)-(1)-C
N-(2)-linker-(3)-FP-(1)-C
N-(2)-FP-(3)-linker-(1)-C
N-(2)-(3)-FP-linker-(1)-C
N-(2)-linker-FP-(3)-linker-(1)-C
N-(2)-linker-(3)FP-linker-(1)-C
N-(1)-FP-(3)-(2)-C
N-(1)-(3)-FP-(2)-C
N-(1)-FP-(3)-linker-(2)-C
N-(1)-(3)-FP-linker-(2)-C
N-(1)-linker-FP-(3)-(2)-C
N-(1)-linker-(3)-FP-(2)-C
N-(1)-linker-FP-(3)-linker-(2)-C
N-(1)-linker-(3)-FP-linker-(2)-C
N-FP-(3)-(1)-(2)-C
N-(3)-FP-(1)-(2)-C
N-FP-(3)-linker-(1)-(2)-C
N-(3)-FP-linker-(1)-(2)-C
N-FP-(3)-(1)-linker-(2)-C
N-(3)-FP-(1)-linker-(2)-C
N-FP-(3)-linker-(1)-linker-(2)-C
N-(3)-FP-linker-(1)-linker-(2)-C
   Nanoparticles Comprising Multiple Copies Fusion Proteins of the Invention and Methods for their Preparation

In a second aspect, the invention relates to a method to prepare nanoparticles comprising multiple copies of the fusion protein according to the first aspect of the invention comprising placing a preparation of said fusion protein in a low salt buffer.

As the person skilled in the art will recognize, “nanoparticles” are microscopic particles whose size is measured in nanometers. The nanoparticles of the invention comprise the nanoparticles that result from the assembly of multiple copies of the fusion protein of the invention as defined in the previous section. In the method for preparing nanoparticles with the fusion proteins of the invention, the preparation of the fusion protein of the invention comprises the monomeric form of the fusion proteins of the invention, which are thermodynamically favored to form non-covalent electrostatic unions and spontaneously aggregate in the conditions of the low salt buffer.

The person skilled in the art will acknowledge that the size of the nanoparticles can be in the range between 1 and 1000 nm, more preferably between 2.5 and 500 nm, even more preferably between 5 and 250 nm, and yet even more preferably between 10 and 100 nm.

It will be understood that the expression “low salt buffer” comprises any buffer solution resulting from the dissolution of one or more salts in water with the capability to moderate changes in pH, wherein the amount of dissolved salt or salts results in an osmolarity lower or equal to that of the physiological fluids, such as the cytoplasm or the extracellular medium, for instance. Thus, the low salt buffer is understood to keep pH and osmolarity inside the range of physiological values and will be used inside the range of physiological temperatures.

The person skilled in the art will recognize that the range of physiological temperatures can oscillate between 15 and 45° C., more preferably between 2° and 40° C., even more preferably between 25 and 39° C., yet even more preferably between 3° and 37° C. The person skilled in the art will also acknowledge that the osmolarity of the low salt buffer will be in the range between 100 and 400 milli-osmoles/L (mOsm/L), preferably between 150 and 350 mOsm/L, more preferably between 200 and 300 mOsm/L, even more preferably between 225 and 275 mOsm/L.

Low salt buffers suitable for the invention, for instance, are the Tris-dextrose buffer (20 mM Tris+5% dextrose, pH 7.4), the Tris-NaCl buffer (20 mM Tris, 500 NaCl, pH 7.4), the PBS-glycerol buffer (phosphate buffered saline, PBS, pH 7.4, which is well known in the art, +10% glycerol), Tris Buffered Saline (TBS)-dextrose (20 mM Tris-HCl buffer pH 7.5, well known in the art, 200NaCl, +5% dextrose), Tris Buffered Saline-Tween 20 (TBST) buffer (10 mM Tris-HCl pH 7.5, 200 mM NaCl, +0.01% Tween 20), or any physiological buffer known in the art with a pH not lower than 6.

In a preferred embodiment of the invention, the low salt buffer of the method of the invention is selected from the group consisting of a carbonate buffer, a Tris buffer and a phosphate buffer.

In a particularly preferred embodiment of the invention, the low salt buffer of the method of the invention is a carbonate buffer that comprises sodium bicarbonate at a concentration between 100 and 300 nM. In another particularly preferred embodiment of the invention, the low salt buffer of the method of the invention is a Tris buffer that comprises Tris at a concentration of between 10 and 30 nM. In another particularly preferred embodiment of the method of the invention, the low salt buffer of the invention is a phosphate buffer that comprises Na₂HPO₄ and NaH₂PO₄ at a total concentration of between 5 mM and 20 mM.

In an even more preferred embodiment of the invention, the low salt buffer of the method of the invention further comprises dextrose and/or glycerol.

In a yet more preferred embodiment of the invention, the low salt buffer of the method of the invention has a pH between 6.5 and 7.5.

In an even yet more preferred embodiment of the invention, the low salt buffer of the method of the invention is selected from the group consisting of
(i) 166 mM NaHCO₃, pH 7.4
(ii) 20 mM Tris, 500 mM NaCl, 5% dextrose, pH 7.4
(iii) 140 mM NaCl, 7.5 mM Na₂HPO₄, 2.5 mM NaH₂PO₄, 10% glycerol, pH 7.4

In another aspect of the invention, the invention relates to nanoparticles comprising multiple copies of the fusion protein of the first aspect of the invention or prepared according to the method or the invention for preparing nanoparticles.

Thus, the nanoparticles of the invention comprise assembled complexes of multiple copies of the fusion proteins of the invention, which result from the electrostatic interaction between regions in their structures favoring their non-covalent binding and coupling in physiological conditions. Since the method of the invention for the preparation of nanoparticles comprises placing a preparation of the fusion protein of the invention in a low salt buffer, it is understood that the nanoparticles thus formed comprise also an assembled complex of multiple copies of the fusion protein.

In a preferred embodiment of the invention, the nanoparticles of the invention have a diameter between 10 and 100 nm.

### Polynucleotide, Vector, and Host Cells of the Invention

In another aspect of the invention, the invention relates to a polynucleotide encoding the fusion protein of the first aspect invention, a vector comprising the aforementioned polynucleotide, and a host cell comprising the aforementioned polynucleotide or the aforementioned vector.

The terms “nucleic acid” and “polynucleotide”, as used herein interchangeably, refer to a polymer composed of nucleotide units (ribonucleotides, deoxyribonucleotides, related naturally occurring structural variants and synthetic non-naturally occurring analogs thereof or combinations thereof) linked via phosphodiester bonds, related naturally occurring structural variants and synthetic non-naturally occurring analogs thereof.

The person skilled in the art will acknowledge that the polynucleotide encodes the polypeptide or protein sequence of the fusion protein of the invention that corresponds to the first aspect of the invention. The polynucleotide of the invention therefore comprises the sequence encoding all of the elements comprised in the fusion protein: the polycationic polypeptide, the intervening peptide region, the positively charged amino acid-rich region, and any other elements that may be part of the fusion protein such as the reporter protein, linkers, and so on and so forth.

It is understood that the nucleic acids or polynucleotides of the invention include coding regions and the adequate regulatory signals for promoting expression in cells to give rise to the biologically active fusion protein.

Generally, nucleic acids containing a coding region will be operably linked to appropriate regulatory sequences. Such regulatory sequence will at least comprise a promoter sequence. As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is only active in specific types of differentiated cells/tissues.

In principle, any promoter can be used for the gene constructs of the present invention provided that said promoter is compatible with the cells in which the polynucleotide is to be expressed. Thus, promoters suitable for the embodiment of the present invention include, without being necessarily limited to, constitutive promoters such as the derivatives of the genomes of eukaryotic viruses such as the polyoma virus, adenovirus, SV40, CMV, avian sarcoma virus, hepatitis B virus, the promoter of the metallothionein gene, the promoter of the herpes simplex virus thymidine kinase gene, retrovirus LTR regions, the promoter of the immunoglobulin gene, the promoter of the actin gene, the promoter of the EF-1alpha gene as well as inducible promoters in which the expression of the protein depends on the addition of a molecule or an exogenous signal, such as the tetracycline system, the NFκB/UV light system, the Cre/Lox system and the promoter of heat shock genes, the regulatable promoters of RNA polymerase II described in WO/2006/135436 as well as tissue-specific promoters.

The polynucleotides of the invention encoding the fusion protein of the invention can be part of a vector. Thus, in another embodiment, the invention relates to a vector comprising a polynucleotide of the invention. A person skilled in the art will understand that there is no limitation as regards the type of vector which can be used because said vector can be a cloning vector suitable for propagation and for obtaining the polynucleotides or expression vectors in different heterologous organisms suitable for purifying the fusion proteins of the invention. Thus, suitable vectors according to the present invention include expression vectors in prokaryotes such as pET (such as pET14b), pUC18, pUC19, Bluescript and their derivatives, mp18, mp19, pBR322, pMB9, ColE1, pCR1, RP4, phages and shuttle vectors such as pSA3 and pAT28, expression vectors in yeasts such as vectors of the type of 2 micron plasmids, integration plasmids, YEP vectors, centromeric plasmids and the like, expression vectors in insect cells such as the pAC series and pVL series vectors, expression vectors in plants such as vectors of expression in plants such as pIBI, pEarleyGate, pAVA, pCAMBIA, pGSA, pGWB, pMDC, pMY, pORE series vectors and the like and expression vectors in superior eukaryotic cells based on viral vectors (adenoviruses, viruses associated to adenoviruses as well as retroviruses and lentiviruses) as well as non-viral vectors such as pSilencer 4.1-CMV (Ambion), pcDNA3, pcDNA3.1/hyg pHCMV/Zeo, pCR3.1, pEFI/His, pIND/GS, pRc/HCMV2, pSV40/Zeo2, pTRACER-HCMV, pUB6/V5-His, pVAXl, pZeoSV2, pCI, pSVL and pKSV-10, pBPV-1, pML2d and pTDT1.

The vector of the invention can be used to transform, transfect, or infect cells which can be transformed, transfected or infected by said vector. Said cells can be prokaryotic or eukaryotic. By way of example, the vector wherein said DNA sequence is introduced can be a plasmid or a vector which, when it is introduced in a host cell, is integrated in the genome of said cell and replicates together with the chromosome (or chromosomes) in which it has been integrated. Said vector can be obtained by conventional methods known by the persons skilled in the art [Sambrook et al., 2001, “Molecular cloning, to Laboratory Manual”, 2nd ed., Cold Spring Harbor Laboratory Press, N.Y. Vol 1-3 a].

Therefore, the invention also relates to a cell comprising a polynucleotide or a vector of the invention, for which said cell has been able to be transformed, transfected or infected with a polynucleotide or vector provided by this invention. The transformed, transfected or infected cells can be obtained by conventional methods known by persons skilled in the art [Sambrook et al., 2001, mentioned above].

Host cells suitable for the expression of the conjugates of the invention include, without being limited to, mammal, plant, insect, fungal and bacterial cells. Bacterial cells include, without being limited to, Gram-positive bacterial cells such as species of the Bacillus, Streptomyces, Listeria and Staphylococcus genera and Gram-negative bacterial cells such as cells of the Escherichia, Salmonella and Pseudomonas genera. Fungal cells preferably include cells of yeasts such as Saccharomyces cerevisiae, Pichia pastoris and Hansenula polymorpha. Insect cells include, without being limited to, Drosophila and Sf9 cells. Plant cells include, among others, cells of crop plants such as cereals, medicinal, ornamental or bulbous plants. Suitable mammal cells in the present invention include epithelial cell lines (human, ovine, porcine, etc.), osteosarcoma cell lines (human, etc.), neuroblastoma cell lines (human, etc.), epithelial carcinomas (human, etc.), glial cells (murine, etc.), hepatic cell lines (from monkey, etc.), CHO (Chinese Hamster Ovary) cells, COS cells, BHK cells, HeLa cells, 911, AT1080, A549, 293 or PER.C6, NTERA-2 human ECC cells, D3 cells of the mESC line, human embryonic stem cells such as HS293, BGVO1, SHEF1, SHEF2, HS181, NIH3T3 cells, 293T, REH and MCF-7 and hMSC cells.

In a preferred embodiment of the invention, the polynucleotide, the vector, and the host cell of the invention are suitable for the expression of the biologically active form of the fusion protein of the invention.

### Uses in Medicine of the Fusion Protein, the Polynucleotide, the Vector, and the Nanoparticle of the Invention

In another aspect, the invention relates to a fusion protein, a polynucleotide, a vector, a host cell or a nanoparticle according to the invention for use in medicine.

It will be understood by the person skilled in the art that by use in medicine, the fusion protein, polynucleotide, vector, host cell, or nanoparticle of the invention can be administered to a patient in order to induce a therapeutic response. The therapeutic response comprises the suppression, reduction or arrest of the causes of the pathological condition or the disease suffered by a patient; the elimination, reduction, arrest or amelioration of the symptoms of the condition or disease; or the extinction, arrest or slowing down of the progression of the condition or disease in the patient.

The person skilled in the art will acknowledge that the fusion protein, polynucleotide, vector, host cell or nanoparticle of the invention suitable for use in medicine may be presented accompanied by a pharmaceutically acceptable carrier. As used herein, the term “pharmaceutically acceptable carrier” means a non-toxic, inert solid, semi-solid or liquid filler, diluent, encapsulating material or formulation auxiliary of any type. Remington's Pharmaceutical Sciences. Ed. by Gennaro, Mack Publishing, Easton, Pa., 1995 discloses various carriers used in formulating pharmaceutical compositions and known techniques for the preparation thereof.

Accordingly, the compositions comprising the fusion protein, polynucleotide, vector, host cell, or nanoparticle of the invention and a pharmaceutically acceptable carrier are pharmaceutical compositions.

The pharmaceutical compositions of this invention can be administered to a patient by any means known in the art including oral and parenteral routes. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

### A—Use of the Fusion Protein, the Polynucleotide, the Vector, the Host Cell, or the Nanoparticle of the Invention in the Treatment of Cancer.

Another embodiment of the invention relates to a fusion protein, a polynucleotide of the invention, the vector of the invention, the host cell of the invention comprising the vector or the polynucleotide and expressing the fusion protein, and the nanoparticle of the invention, or their corresponding pharmaceutical compositions, wherein the polycationic peptide is a sequence capable of specifically interacting with a receptor on a cell surface which is capable of promoting the internalization of the fusion protein into the cell, wherein said cell expressing the receptor is a tumor cell present in cancer, and wherein the intervening polypeptide region is an antitumor peptide, for use in the treatment of cancer.

As used herein, the terms “treat”, “treatment” and “treating” refer to the reduction or amelioration of the progression, severity and/or duration of cancer, or the amelioration of one or more symptoms (preferably, one or more discernible symptoms) of cancer. The terms “treat”, “treatment” and “treating” also refer to the amelioration of at least one measurable physical parameter of cancer, such as growth of a tumor, not necessarily discernible by the patient. Furthermore, “treat”, “treatment” and “treating” refer also to the inhibition of the progression of cancer, either physically by, e.g., stabilization of a discernible symptom, physiologically by, e.g., stabilization of a physical parameter, or both. “Treat”, “treatment” and “treating” may refer, too, to the reduction or stabilization of tumor size or cancerous cell count.

The term “cancer” refers to a group of diseases involving abnormal, uncontrolled cell growth and proliferation (neoplasia) with the potential to invade or spread (metastasize) to other tissues, organs or, in general, distant parts of the organism; metastasis is one of the hallmarks of the malignancy of cancer and cancerous tumors. The abnormal growth and/or proliferation of cancerous cells is the result of a combination of genetic and environmental factors that alter their normal physiology. The growth and/or proliferation abnormalities of cancerous cells result in physiological disorders and, in many cases, death of the individual, due to the dysfunctionality or loss of functionality of the cell types, tissues and organs affected.

The term “cancer” includes, but is not restricted to, cancer of the breast, heart, small intestine, colon, spleen, kidney, bladder, head, neck, ovaries, prostate gland, brain, pancreas, skin, bone, bone marrow, blood, thymus, womb, testicles, hepatobiliary system and liver; in addition to tumors such as, but not limited to, adenoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, hepatobiliary cancer, osteosarcoma, retinoblastoma, rhabdomyosarcoma, sarcoma and teratoma. Furthermore, this term includes acrolentiginous melanoma, actinic keratosis adenocarcinoma, adenoid cystic carcinoma, adenomas, adenosarcoma, adenosquamus carcinoma, astrocytic tumors, Bartholin gland carcinoma, basal cell carcinoma, bronchial gland carcinoma, capillary carcinoid, carcinoma, carcinosarcoma, cholangiocarcinoma, cystadenoma, endodermal sinus tumor, endometrial hyperplasia, endometrial stromal sarcoma, endometrioid adenocarcinoma, ependymal sarcoma, Ewing sarcoma, focal nodular hyperplasia, germ cell tumors, glioblastoma, glucagonoma, hemangioblastoma, hemagioendothelioma, hemagioma, hepatic adenoma, hepatic adenomastosis, hepatocellular carcinoma, hepatobilliary cancer, insulinoma, intraepithelial neoplasia, squamous cell intraepithelial neoplasia, invasive squamous-cell carcinoma, large cell carcinoma, leiomyosarcoma, melanoma, malignant melonoma, malignant mesothelial tumor, meduloblastoma, medulloepithelioma, mucoepidermoid carcinoma, neuroblastoma, neuroepithelial adenocarcinoma, nodular melanoma, osteosarcoma, papillary serous adenocarcinoma, pituitary tumors, plasmacytoma, pseudosarcoma, pulmonary blastoma, renal cell carcinoma, retinoblastoma, rhabdomyosarcoma, sarcoma, serous carcinoma, microcytic carcinoma, soft tissue carcinoma, somatostatin secreting tumor, squamous carcinoma, squamous cell carcinoma, undifferentiated carcinoma, uveal melanoma, verrucous carcinoma, vipoma, Wilm tumor, intracerebral cancer, head and neck cancer, rectal cancer, astrocytoma, glioblastoma, microcytic cancer and non-microcytic cancer, metastatic melanoma, androgen-independent metastatic prostate cancer, androgen-dependent metastatic prostate cancer and breast cancer.

Thus, in a preferred embodiment of the invention, the antitumor peptide of the fusion protein, the polynucleotide, the vector, the host cell, or the nanoparticle of the invention is selected from the group consisting of
(i) a cytotoxic polypeptide,
(ii) an antiangiogenic polypeptide,
(iii) a polypeptide encoded by a tumor suppressor gene,
(iv) a pro-apoptotic polypeptide,
(v) a polypeptide having anti-metastatic activity,
(vi) a polypeptide encoded by a polynucleotide which is capable of activating the immune response towards a tumor,
(vii) a chemotherapy agent,
(viii) an antiangiogenic molecule and
(ix) a polypeptide encoded by a suicide gene.

In a more preferred embodiment of the invention, the antitumor peptide of the fusion protein, the polynucleotide, the vector, the host cell, or the nanoparticle of the invention is selected from the group consisting of the BH3 domain of BAK, PUMA, GW-H1, the Diphtheria toxin, the Pseudomonas exotoxin and Ricin. In a further preferred embodiment of the invention, the antitumor peptide of the fusion protein, the polynucleotide, the vector, the host cell, or the nanoparticle of the invention is a truncated form or a mutant of the peptide selected from the group indicated just before, preferably from the group consisting of the Diphtheria toxin, the Pseudomonas exotoxin and Ricin. Preferred sequences of said peptides are indicated above in the “Intervening polypeptide region” section.

In an even more preferred embodiment of the invention, the polycationic peptide of the fusion protein, the polynucleotide, the vector, the host cell or the nanoparticle of the invention is a CXCR4 ligand, and the cancer targeted to be treated with the fusion protein, the polynucleotide, the vector, the host cell, or the nanoparticle of the invention is characterized by comprising cells which express the CXCR4 receptor.

In a yet more preferred embodiment of the invention, the CXCR4 ligand of the fusion protein, the polynucleotide, the vector, the host cell, or the nanoparticle of the invention is selected from the group comprising the T22 peptide, the V1 peptide, the CXCL12 peptide, the vCCL2 peptide or a functionally equivalent variant thereof.

In another more preferred embodiment of the invention, the cancer to be treated with the fusion protein, the polynucleotide, the vector, the host cell, or the nanoparticle of the invention is selected from the group consisting of pancreatic and colorectal cancer.

The protein CD44 is another well-known key regulator of progression and metastasis in cancer cells (as reviewed in Senbanjo, L. T. & Chellaiah, M. A. 2017. Front. Cell Dev. Biol. 5:18).

Thus, in another preferred embodiment of the invention, the cancer to be treated with the fusion protein, the polynucleotide, the vector, the host cell, or the nanoparticle of the invention is characterized by the expression of CD44.

Another more preferred embodiment of the invention relates to the fusion protein, the polynucleotide, the vector, the host cell, or the nanoparticle of the invention, for use in the treatment of cancer, wherein the cancer is characterized by the expression of CD44, wherein the intervening region polypeptide is an antitumor peptide selected from one of the groups already listed, wherein the polycationic peptide region is a CD44 ligand, and wherein the CD44 ligand is A5G27 or FNI/II/V.

Another even more preferred embodiment of the invention relates to the fusion protein, the polynucleotide, the vector, the host cell, or the nanoparticle of the invention, for use in the treatment of cancer, wherein the cancer is characterized by the expression of CD44, wherein the intervening region polypeptide is an antitumor peptide, wherein the polycationic peptide region is a CD44 ligand selected between A5G27 and FNI/II/V, and wherein the cancer is colon, liver, prostate or breast cancer.

Another preferred embodiment of the invention relates to the fusion protein, the polynucleotide, the vector, the host cell, or the nanoparticle of the invention of the invention, wherein the polycationic peptide is a peptide capable of crossing the blood-brain barrier, and wherein the intervening region polypeptide is an antitumor peptide, for use in the treatment of cancer of the central nervous system.

Another more preferred embodiment of the invention relates to the fusion protein, the polynucleotide, the vector, the host cell, or the nanoparticle of the invention, wherein the polycationic peptide is a peptide capable of crossing the blood-brain barrier, and wherein the antitumor peptide is selected from one of the groups already listed, for use in the treatment of a cancer of the central nervous system.

An even more preferred embodiment of the invention relates to the fusion protein, the polynucleotide, the vector, the host cell, or the nanoparticle of the invention, wherein the polycationic peptide is a peptide capable of crossing the blood-brain barrier selected from the group consisting of Seq-1-7, Seq-1-8, and Angiopep-2-7, and wherein the antitumor peptide is selected from one of the groups already listed, for use in the treatment of a cancer of the central nervous system.

A yet even more preferred embodiment of the invention relates to the fusion protein, the polynucleotide, the vector, the host cell, or the nanoparticle of the invention, wherein the polycationic peptide is a peptide selected from the group consisting of Seq-1-7, Seq-1-8, and Angiopep-2-7, and wherein the antitumor peptide is selected from one of the groups already listed, for use in the treatment of a cancer of the central nervous system, wherein the cancer central nervous system is a glioma.

### B—Use of the Fusion Protein, the Polynucleotide, the Vector, the Host Cell, or the Nanoparticle of the Invention in the Treatment of Bacterial Infections

Another embodiment of the invention relates to the fusion protein, the polynucleotide, the vector, the host cell, or the nanoparticle of the invention for use in the treatment of a disease caused by a bacterial infection.

As used herein, the terms “treat”, “treatment” and “treating” refer to the reduction or amelioration of the progression, severity and/or duration of a bacterial infection, or the amelioration of one or more symptoms (preferably, one or more discernible symptoms) of a bacterial infection. The terms “treat”, “treatment” and “treating” also refer to the amelioration of at least one measurable physical parameter of a bacterial infection, such as presence of bacterial toxins, not necessarily discernible by the patient. Furthermore, “treat”, “treatment” and “treating” refer also to the inhibition of the progression of a bacterial infection, either physically by, e.g., stabilization of a discernible symptom, physiologically by, e.g., stabilization of a physical parameter, or both. “Treat”, “treatment” and “treating” may refer, too, to the reduction or stabilization of the bacterial cell count.

The term “bacteria”, as used herein, refers to Prokaryotes of the domain Bacteria. Non-limiting examples of bacterial genera that may be used in the method of the present invention include: Actinomyces, Bacillus, Bacteroides, Bartonella, Bordetella, Borrelia, Brucella, Burkholderia, Campylobacter, Chlamydia, Clostridium, Corynebacterium, Coxiella, Ehrlichia, Enterococcus, Escherichia, Francisella, Haemophilus, Helicobacter, Klebsiella, Legionella, Leptospira, Listeria, Moraxella, Mycobacterium, Mycoplasma, Neisseria, Nocardia, Pseudomonas, Rickettsia, Salmonella, Shigella, Staphylococcus, Streptobacillus, Streptococcus, Treponema, Ureaplasma, Vibrio and Yersinia. Individual Prokaryotes of the domain Bacteria are denominated bacterium.

The invention contemplates the suitability of the fusion protein, the polynucleotide, the vector, the host cell, or the nanoparticle for the treatment of infections of bacteria such as Neisseria spp, including N. gonorrhea and N. meningitides, Streptococcus pyogenes Streptococcus agalactiae, Streptococcus mutans; Haemophilus ducreyi; Moraxella spp., including M. catarrhalis, also known as Branhamella catarrhalis Bordetella spp., including B. pertussis, B. parapertussis and B. bronchiseptica, Mycobacterium spp., including M. tuberculosis, M. bovis, M. leprae, M. avium, M. paratuberculosis, M. smegmatis; Legionella spp, including L. pneumophila, Escherichia spp., including enterotoxic E. coli, enterohemorragic E. coli and enteropathogenic E. coli, Vibrio spp, including V. cholera, Shigella spp., including S. sonnei, S. dysenteriae, S. flexnerii; Yersinia spp., including Y. enterocolitica, Y. pestis, Y. pseudotuberculosis; Campylobacter spp., including C. jejuni, Salmonella spp., including S. typhi, S. enterica and S. bongori; Listeria spp., including L. monocytogenes; Helicobacter spp., including H. pylori, Pseudomonas spp., including P. aeruginosa; Staphylococcus spp., including S. aureus, S. epidermidis; Enterococcus spp., including E. faecalis, E. faecium; Clostridium spp., including C. tetani, C. botulinum, C. dificile, Bacillus spp., including B. anthracis; Corynebacterium spp., including C. diphtheria, Borrelia spp., including B. burgdorferi, B. garinii, B. afzelii, B. andersonii, B. hermsii; Ehrlichia spp., including E. equi and the agent of the Human Granulocytic Ehrlichiosis; Rickettsia spp., including R. rickettsii; Chlamydia spp., including C. trachomatis, Chlamydia pneumoniae, C. psittaci; Leptospira spp., including L. interrogans; Treponema spp., including T. pallidum, T. denticola, T. hyodysenteriae, Mycobacterium tuberculosis, Streptococcus spp., including S. pneumoniae, Haemophilus spp., including H. influenzae type B, and non typeable H. influenza, among others and without limitation.

### C—Use of the Fusion Protein, the Polynucleotide, the Vector, the Host Cell, or the Nanoparticle of the Invention in the Treatment of Viral Infections

Another embodiment of the invention, relates to the fusion protein, the polynucleotide, the vector, the host cell, or the nanoparticle of the invention, wherein the polycationic peptide is capable of specifically interacting with a receptor on the cell surface of a cell infected by a virus causing an infection; and wherein the intervening polypeptide region is an antiviral agent, for use in the treatment of a disease caused by a viral infection.

As used herein, the terms “treat”, “treatment” and “treating” refer to the reduction or amelioration of the progression, severity and/or duration of a viral infection, or the amelioration of one or more symptoms (preferably, one or more discernible symptoms) of a viral infection. The terms “treat”, “treatment” and “treating” also refer to the amelioration of at least one measurable physical parameter of a bacterial infection, such as viral titer, not necessarily discernible by the patient. Furthermore, “treat”, “treatment” and “treating” refer also to the inhibition of the progression of a viral infection, either physically by, e.g., stabilization of a discernible symptom, physiologically by, e.g., stabilization of a physical parameter, or both. “Treat”, “treatment” and “treating” may refer, too, to the reduction or stabilization of the viral titer.

The term “virus”, as used herein, refers to a small infectious agent that can replicate only inside the living cells of organisms. Non-limiting examples of viral families that may be used in the method of the present invention include Adenoviridae, African swine fever-like viruses, Arenaviridae, Arteriviridae, Astroviridae, Baculoviridae, Birnaviridae, Bunyaviridae, Caliciviridae, Circoviridae, Coronaviridae, Deltavirus, Filoviridae, Flaviviridae, Hepadnaviridae, Hepeviridae, Herpesviridae, Orthomyxoviridae, Paramyxoviridae, Picomaviridae, Poxyviridae, Reoviridae, Retroviridae and Rhabdoviridae.

Examples of viral infections that the fusion protein, the polynucleotide, the vector, the host cell, or the nanoparticle of the invention are suitable to treat include those of Human Immunodeficiency Virus (HIV-1), human herpes viruses, like HSV1 or HSV2, cytomegalovirus, especially Human, Epstein Barr virus, Varicella Zoster Virus, hepatitis virus such as hepatitis B virus, hepatitis C virus, paramyxoviruses such as Respiratory Syncytial virus, parainfluenza virus, rubella virus, measles virus, mumps virus, human papilloma viruses, flaviviruses (e.g. Yellow Fever Virus, Dengue Virus, Tick-borne encephalitis virus, Japanese Encephalitis Virus), Influenza virus, rotavirus, and the like.

In an even more preferred embodiment of the invention, the antiviral agent of the fusion protein, the polynucleotide, the vector, the host cell, or the nanoparticle of the invention is selected from the group consisting of
(i) A cytotoxic polypeptide,
(ii) A pro-apoptotic polypeptide,
(iii) A polypeptide encoded by a suicide gene; and
(iv) An antiretroviral polypeptide

Cytotoxic polypeptides (i), pro-apoptotic polypeptides (ii) and polypeptides encoded by a suicide gene have already been discussed in the section corresponding to the fusion protein.

Antiretroviral agents are one subtype of the antiviral class of antimicrobials. Antiretroviral agents are used specifically for treating viral infections caused by retroviruses. Retroviruses comprise the Retroviridae family of viruses, which includes genera such as Alpharetrovirus, Betaretrovirus, and Lentivirus, to name a few. They are characterized by being single-stranded, positive-sense RNA-genome viruses. Retroviruses generate, through their own reverse transcriptase, a double stranded DNA copy of their genome that integrates in the genome of their host cell. The person skilled in the art will recognize that “antiretroviral agents” comprises any molecules or compounds capable of interfering with the normal replication cycle of a retrovirus at any of its stages. Thus, an antiretroviral polypeptide (iv), as used herein refers to a polypeptide with antiretroviral properties.

Antiretroviral polypeptides suitable for the invention are, for instance, “entry inhibitors”, also known as “fusion inhibitors”, peptides which interfere with the binding, fusion and entry of the retrovirus to the host cell. Examples of this group are efuvirtide, a biomimetic peptide that competes with the fusion machinery of HIV-1, and peptide T, a peptide that blocks chemokine receptors CCR2 and CCR5.

Also comprised as entry inhibitors are antibodies specific against the receptors used by retroviruses to fuse with the cell. Non-limiting examples of these receptors suitable to be blocked with antibodies, are CD4, CCR2, CCR5, and CXCR4.

The term “antibody”, as used herein, refers to a glycoprotein that exhibits specific binding activity for a particular protein, which is referred to as “antigen”. The term “antibody” comprises whole monoclonal antibodies or polyclonal antibodies, or fragments thereof, and includes human antibodies, humanised antibodies, chimeric antibodies and antibodies of a non-human origin. “Monoclonal antibodies” are homogenous, highly specific antibody populations directed against a single site or antigenic “determinant”. “Polyclonal antibodies” include heterogeneous antibody populations directed against different antigenic determinants.

As used herein, the antibodies suitable for the invention encompass not only full length antibodies (e.g., IgG), but also antigen-binding fragments thereof, for example, Fab, Fab′, F(ab′)2, Fv fragments, human antibodies, humanised antibodies, chimeric antibodies, antibodies of a non-human origin, recombinant antibodies, and polypeptides derived from immunoglobulins produced by means of genetic engineering techniques, for example, single chain Fv (scFv), diabodies, heavy chain or fragments thereof, light chain or fragment thereof, VH or dimers thereof, VL or dimers thereof, Fv fragments stabilized by means of disulfide bridges (dsFv), molecules with single chain variable region domains (Abs), minibodies, scFv-Fc, and fusion proteins comprising an antibody, or any other modified configuration of the immunoglobulin molecule that comprises an antigen recognition site of a desired specificity. The antibody of the invention may also be a bispecific antibody. An antibody fragment may refer to an antigen binding fragment. An antibody includes an antibody of any class, namely IgA, IgD, IgE, IgG (or sub-classes thereof), and IgM, and the antibody need not be of any particular class.

Thus, a yet more preferred embodiment of the invention relates to the fusion protein, polynucleotide, vector, host cell, or nanoparticle of the invention, wherein the polycationic peptide is a CXCR4 ligand, and wherein the cell is an HIV-infected cell, for use in the treatment of HIV infection.

A yet even more preferred embodiment of the invention relates to the fusion protein, the polynucleotide, the vector, the host cell, or nanoparticle of the invention, wherein the CXCR4 ligand is selected from the group consisting of SEQ ID NO: 5, SEQ ID NO: 6, SEQ ID NO: 7 and SEQ ID NO: 8 or a functionally equivalent variant thereof for use in the treatment of a viral infection.

### D—Use of the Fusion Protein, the Polynucleotide, the Vector, the Host Cell, or the Nanoparticle of the Invention in the Treatment of Neurodegenerative Diseases

Protein aggregation is a biological phenomenon which results from the accumulation of misfolded proteins, whether intra- or extracellularly. The resulting protein aggregates can originate diseases and, in fact, it has been found their involvement in a wide range of diseases known as amyloidoses. The amyloidoses comprise several well-studied neurodegenerative diseases, like ALS, Alzheimer's, Parkinson's and prion disease.

Aggregation occurs due to errors in the physiological folding of proteins into their natural three-dimensional conformation, which is the most thermodynamically favorable (also known as “native state”). The folding process is driven by the tendency of hydrophobic portions of the protein to shield itself from the hydrophilic environment of the cell by burying into the interior of the protein. Thus, the exterior of a protein is typically hydrophilic, whereas the interior is typically hydrophobic. Protein structures are then stabilized by non-covalent electrostatic interactions and disulfide bonds, well known to the person skilled in the art, that originate the secondary and tertiary structures of the proteins.

The errors that lead to misfolding or unfolding of the protein may be originated by alterations in the amino acid sequence of the protein. Should these errors not be corrected, for instance through “chaperone proteins” (as the person skilled in the art will know, chaperone proteins or “chaperones” are proteins which assist as a scaffolding for the correct folding of other proteins into their correct conformation and tertiary or tridimensional structure), the misfolded or unfolded proteins will aggregate due to the natural interaction of their hydrophobic regions with one another as a way to limit their exposure to the hydrophilic environment of the cells [Roberts, C. J., 2007. Biotechnology & Bioengineering, 98(5):927-938].

Thus, another embodiment of the invention relates to the fusion protein, the polynucleotide, the vector, or the nanoparticle of the invention, wherein the polycationic peptide is a peptide capable of crossing the blood-brain barrier, and wherein the intervening polypeptide region is a chaperone or an inhibitor of protein aggregation, for use in the treatment of a neurodegenerative disease.

Suitable chaperones or inhibitors of protein aggregation are as defined above. Diseases that can be treated using the fusion proteins, nanoparticles, vectors or host cells according to the invention include Alzheimer's disease, Pick's disease, Alphal-antitrypsin deficiency, Parkinson's disease and other synucleinopathies, Creutzfeldt-Jakob disease, Retinal ganglion cell degeneration in glaucoma, Cerebral β-amyloid angiopathy, Prion diseases, Tauopathies, Frontotemporal lobar degeneration, Type II diabetes, Amyotrophic lateral sclerosis, Huntington's disease and other trinucleotide repeat disorders, Familial Danish dementia, Familial English dementia, Hereditary cerebral hemorrhage with amyloidosis, Alexander disease, Seipinopathies, Familial amyloidotic neuropathy, Senile systemic amyloidosis, Lysozyme amyloidosis, Fibrinogen amyloidosis, Dialysis amyloidosis, Inclusion body myositis/myopathy, Cataracts, Retinitis pigmentosa with rhodopsin mutations, Medullary thyroid carcinoma, Cardiac atrial amyloidosis, Pituitary prolactinoma, Hereditary lattice corneal dystrophy, Cutaneous lichen amyloidosis, Mallory bodies, Corneal lactoferrin amyloidosis, Pulmonary alveolar proteinosis, Odontogenic tumor amyloid, Seminal vesicle amyloid, Apolipoprotein C2 amyloidosis, Apolipoprotein C3 amyloidosis, Lect2 amyloidosis, Insulin amyloidosis, Galectin-7 amyloidosis (primary localized cutaneous amyloidosis), Corneodesmosin amyloidosis, Enfuvirtide amyloidosis, Cystic Fibrosis, Sickle cell disease, Hereditary cerebral hemorrhage with amyloidosis, AL amyloidosis AH amyloidosis, AA amyloidosis, Aortic medial amyloidosis, ApoAI amyloidosis, ApoAII amyloidosis, ApoAIV amyloidosis and Familial amyloidosis of the Finnish type.

Accordingly, a preferred embodiment of the invention relates to the fusion protein, the polynucleotide, the vector, the host cell, or the nanoparticle of the invention, wherein the intervening polypeptide region is a chaperone or an inhibitor of protein aggregation, for use in the treatment of a neurodegenerative disease, wherein the polycationic peptide capable of crossing the blood-brain barrier is selected from the group consisting of Seq-1-7, Seq-1-8, and Angiopep-2-7.

The invention is described below by way of the following examples which are to be taken as merely illustrative and not limiting the scope of the invention.

### EXAMPLES

### Materials and Methods Regarding Fusion Proteins T22-BAK-GFP-H6, T22-GFP-H6, T22-GWH1-GFP-H6, and T22-PUMAGFP-H6

### Protein Design, Production and Purification

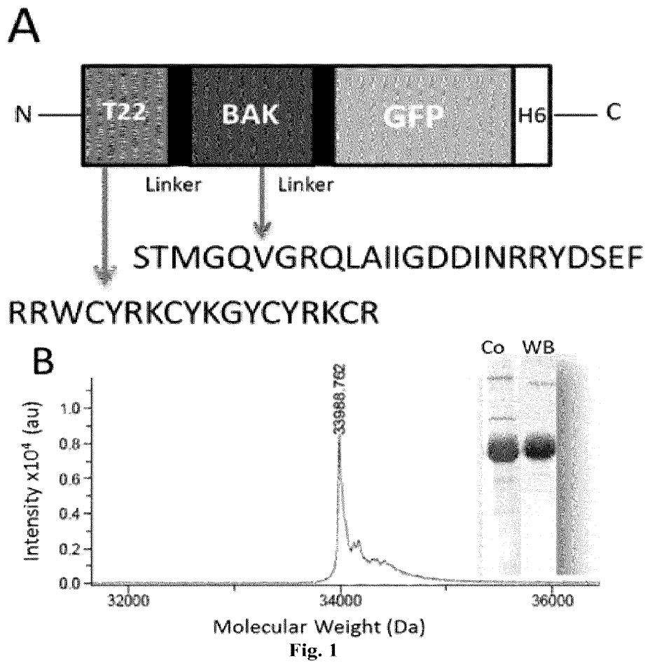

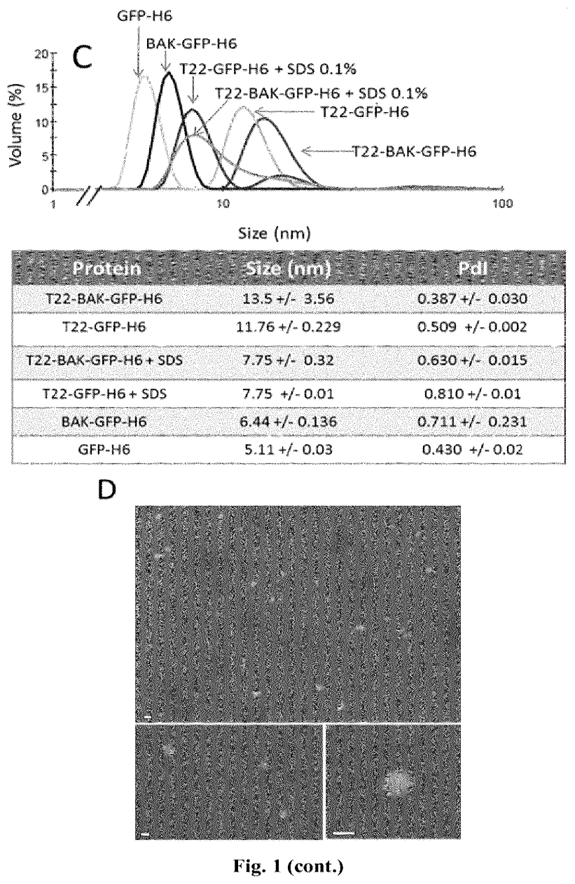

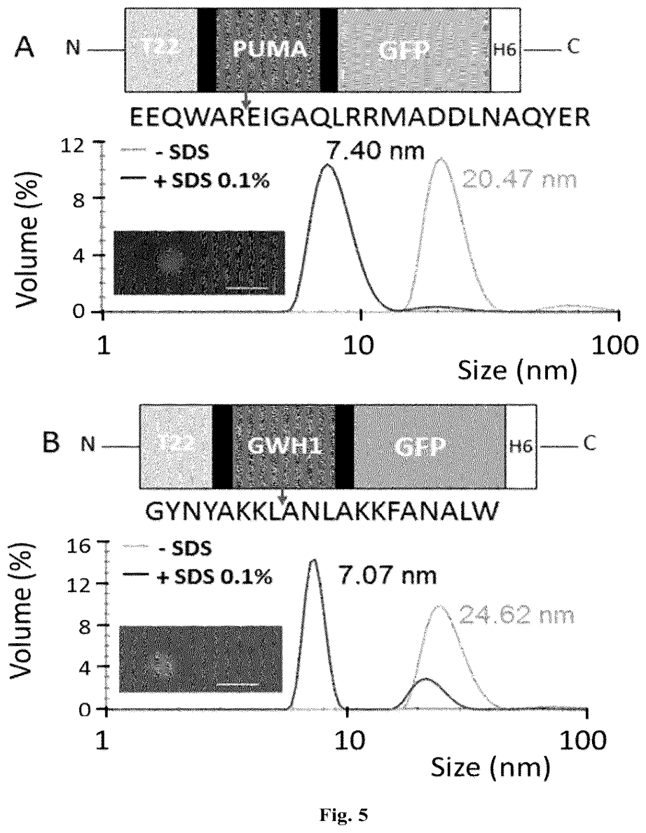

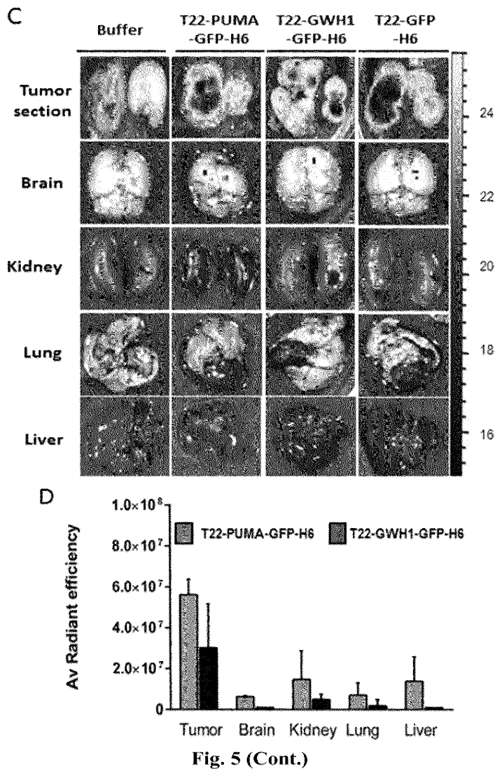

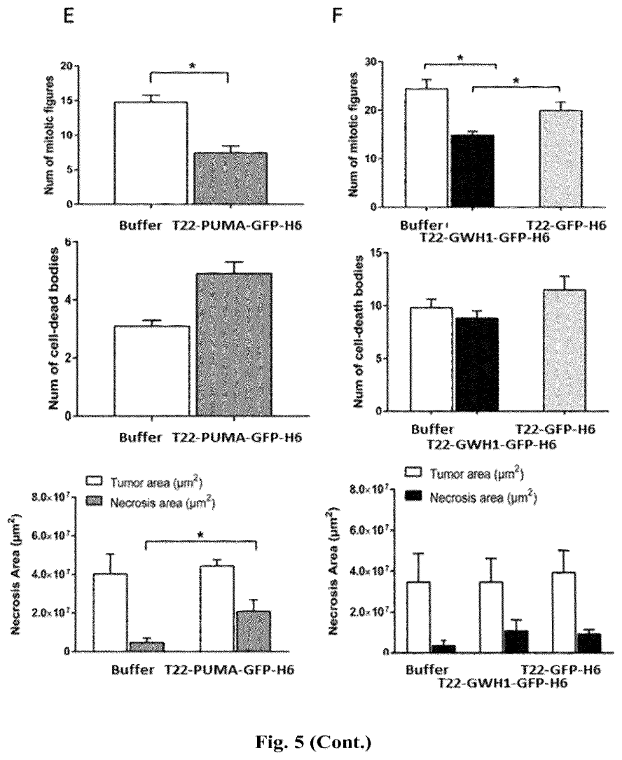

The engineered fusion proteins were named according to their modular organization (FIGS. 1, 5; T22-BAK-GFP-H6, T22-GFP-H6, T22-GWH1-GFP-H6, and T22-PUMAGFP-H6). Synthetic genes were designed in house and obtained from GeneArt inserted into the prokaryotic expression pET-22b vector. The encoded proteins were produced in plasmid bearing Escherichia coli Origami B (BL21, OmpT-, Lon-, TrxB-, Gor-, Novagen) cells, cultured in 2 L-shaker flasks with 500 ml of LB medium with 100 μg/ml ampicillin, 15 μg/ml kanamycin and 12.5 μg/ml tetracycline at 37° C. Recombinant gene expression was induced at an OD550 around 0.5-0.7 upon the addition of 0.1 mM isopropyl-β-d-thiogalactopyronaside (IPTG) and then, bacterial cells were kept growing 3 hours at 37° C. for the T22-BAK-GFP-H6 fusion protein production and overnight at 20° C. for T22-GFP-H6, T22-GWH1-GFP-H6 and T22-PUMA-GFP-H6 production.

Bacterial cells were then harvested by centrifugation at 5000 g for 15 min at 4° C. and resuspended in wash buffer (20 mM Tris-HCl, 500 mM NaCl, 10 mM imidazol, pH 8.0) in the presence of EDTA-free protease inhibitor (Complete EDTA-Free; Roche, Basel, Switzerland). Cells were disrupted at 1200 psi in a French Press (Thermo FA-078A) and lysates were centrifuged for 45 min (15,000 g at 4° C.).

All proteins were purified by His-tag affinity chromatography using HiTrap Chelating HP 1 ml columns (GE Healthcare, Piscataway, NJ, USA) by AKTA purifier FPLC (GE Healthcare). After filtering the soluble fraction, samples were loaded onto the column and washed with 10 column volumes of wash buffer. Elution was achieved by a linear gradient of 20 mM Tris-HCl, 500 mM NaCl, 500 mM imidazole, pH 8.0 and purified fractions were collected and analyzed by SDS-PAGE and Western Blotting with anti-His monoclonal antibody (Santa Cruz Biotechnology, Heidelberg, Germany) to observe the protein of interest.

Proteins were dialyzed overnight at 4° C., against sodium bicarbonate buffer with salt (166 mM NaHCO3 pH 7.4+333 mM NaCl). These buffers were the final solvents for further experiments. Protein integrity and purity were checked by mass spectrometry (MALDI-TOF) and quantified by Bradford's assay.

### Fluorescence Determination, Dynamic Light Scattering (DLS) and Field Emission Scanning Electron Microscopy (FESEM)

The fluorescence of the fusion proteins was determined in a Varian Cary Eclipse fluorescence spectrophotometer (Agilent Technologies, Palo Alto, CA, USA) at 510 nm using an excitation wavelength of 450 nm. Volume size distribution of nanoparticles and monomeric GFP protein fusions were determined by DLS at 633 nm (Zetasizer Nano ZS, Malvern Instruments Limited, Malvern, UK).

For fluorescence determination, protein samples were diluted in the corresponding storage buffer to 0.5 mg/ml, in 100 μl final volume. For DLS analyses, proteins (stored at −80° C.) were thawed and 50 μl of each sample was used. Field emission scanning electron microscopy (FESEM) qualitative analyses were performed with Zeiss Merlin (Zeiss, Oberkochen, Germany) field emission scanning electron microscope operating at 1 kV and equipped with a high resolution in-lens secondary electron detector. Microdrops of diluted purified proteins were deposited onto silicon wafer surfaces (Ted Pella, Reading, CA, USA), air-dried and immediately observed.

### Cell Culture and Flow Cytometry

The CXCR4+ HeLa cell line (ATCC-CCL-2) was cultured in Eagle's Minimum Essential Medium (Gibco, Rockville, MD, USA) supplemented with 10% fetal calf serum (Gibco®), and incubated at 37° C. and 5% CO2 in a humidified atmosphere. Meanwhile SW1417 cell line was maintained in Dulbecco's Modified Eagle's Medium (DMEM: Gibco® GlutaMAX™, Thermo Fisher Scientific, Waltham, MA, USA) supplemented with 10% fetal calf serum (Gibco®), and incubated at 37° C. and 10% CO2 in a humidified atmosphere. HeLa and SW1417 cell lines were cultured on 24-well plate at 3×10⁴ and 12×10⁴ cells/well respectively for 24 h until reaching 70% confluence.

Nanoparticles and monomeric proteins were added at different concentrations (ranging from 0.1 to 2 μM) to the cell culture in the presence of Optipro medium (Gibco®) 24 h before the flow cytometry analysis. Cell samples were analyzed on a FACSCanto system (Becton Dickinson, Franklin Lakes, NJ, USA) using a 15 W air-cooled argon-ion laser at 488 nm excitation. GFP fluorescence emission was measured with a detector D (530/30 nm band pass filter) after treatment with 1 mg/ml trypsin (Gibco®) for 15 min.

Specific internalization of nanoparticles was measured using AMD3100/CXCR4+ inhibitor (octahydrochloride hydrate, Sigma-Aldrich, Steinheim, Germany). For this experiment, T22-BAK-GFP-H6 was labeled with ATTO488 (41698, Sigma-Aldrich) during 1 h in darkness at room temperature to obtain a more fluorescent protein. T22-BAK-GFP-H6-ATTO488 was added at 25 nM during 1 h of incubation in presence of AMD3100 at 1:10 ratio.

### Confocal Microscopy

HeLa cells were grown on Mat-Tek culture dishes (MatTek Corporation, Ashland, MA, USA). Medium was removed and cells were washed with DPBS, OptiPro medium supplemented with L-glutamine and proteins were added 24 h before staining at 2 μM. Nuclei were labelled with 0.2 μg/ml Hoechst 33342 (Molecular Probes, Eugene, OR, USA) and the plasma membranes with 2.5 μg/ml CellMask™ Deep Red (Molecular Probes) in darkness for 10 min. Live cells were recorded by TCS-SP5 confocal laser scanning microscopy (Leica Microsystems, Heidelberg, Germany) using a Plan Apo 63×/1.4 (oil HC×PL APO lambdablue) objective.

To determine the location of particles inside the cell, stacks of 10-20 sections were collected at 0.5 m Z-intervals with a pinhole setting of 1 Airy unit. Images were processed and the 3-D reconstruction was generated using Imaris version 7.2.1.0 software (Bitplane, Zurich, Switzerland).

### Biodistribution

Five-week-old female Swiss nu/nu mice weighing between 18 and 20 g (Charles River, L'Arbresle, France) and maintained in SPF conditions, were used for in vivo studies. All the in vivo procedures were approved by the Hospital de Sant Pau Animal Ethics Committee and performed according to European Council directives.

To generate the subcutaneous (SC) mouse model, we obtained 10 mg of SP5 CCR tumor tissue from donor animals and implanted subcutaneously in the subcutis of swiss nu/nu mice. When tumors reached 500 mm3 approximately, mice were randomly allocated and administered with T22-BAK-GFP-H6, BAK-GFP-H6 and T22-GFP-H6 nanoparticles at 330 μg/mouse dose.

Short (2 and 5 h) and long times (24 and 48 h) were assayed to explore the biological effects of the administered nanoparticles. For that, mice were euthanized and tumor and brain, pancreas, lung and heart, kidney, liver and bone marrow were collected and examined separately for ex-vivo GFP fluorescence in an IVIS® Spectrum equipment (PerkinElmer Inc, Waltham, MA, USA). The fluorescent signal (FLI) was first digitalized, displayed as a pseudocolor overlay and expressed as radiant efficiency. The FLI ratio was calculated dividing the FLI signal from the protein-treated mice by the FLI auto-fluorescent signal of control mice.

Finally, all organs were collected and fixed with 4% formaldehyde in phosphate-buffered solution for 24 h. These samples were then embedded in paraffin for histological and immunohistochemical analyses as well as for determination of mitotic and apoptotic index and necrosis evaluation.

### Histopathology and Immunohistochemistry Analyses

Four-micrometer-thick sections were stained with hematoxylin and eosin (H&E), and a complete histopathological analysis was performed by two independent observers. The presence and location of the His tag in the protein materials and of the proteolyzed PARP and the active cleaved-Caspase 3 protein in tissue sections were assessed by immunohistochemistry using the DAKO immunosystem equipment and standard protocols. A primary antibody against the His tag (1:1000; MBL International, Woburn, MA, USA), anti-PARP p85 fragment pAb (1:300; Promega, Madison, WI, USA) or anti-active caspase 3 antibody (1:300, BD PharMigen, San Diego, CA, USA) were incubated for 25 min after incubation with the secondary antibody in tumor tissues at 2, 5, 24 and, 48 h. The number of stained cells was quantified by two independent blinded counters who recorded the number of positive cells per 10 high-power fields (magnification 400×). Representative pictures were taken using Cell∧ B software (Olympus Soft Imaging v 3.3, Nagano, Japan).

### Assessment of Mitotic, Apoptotic, Necrotic Rates

Tumor slices were also processed to assess proliferation capacity by counting the number of mitotic figures per ten high-power fields (magnification ×400) in H&E stained tumors. Apoptotic induction was evaluated by the presence of cell death bodies in H&E and also by Hoechst staining in tumor slices. Hoechst 33258 (Sigma-Aldrich, Steinheim, Germany) staining was performed in Triton X-100 (0.5%) permeabilized sections. Slides were then stained with Hoechst 33258 (1:5000 in PBS) for 1 h, rinsed with water, mounting and analyzed under fluorescence microscope (λex=334 nm/λem=465 nm).

The number of apoptotic bodies was quantified by two independent blinded recording the number of condensed and/or defragmented nuclei per 10 high-power fields (magnification 400×). Necrosis area in tumors was quantified using Cell∧ B software at 15× magnification and representative pictures were taken using the same Cell∧ B software at 400× magnification.

Materials and Methods Regarding the Protein Nanoparticles Based on Diptheria Toxin (DITOX) and Pseudomonas aeruginosa Exotoxin (PE24)

### Protein Design, Production and Purification

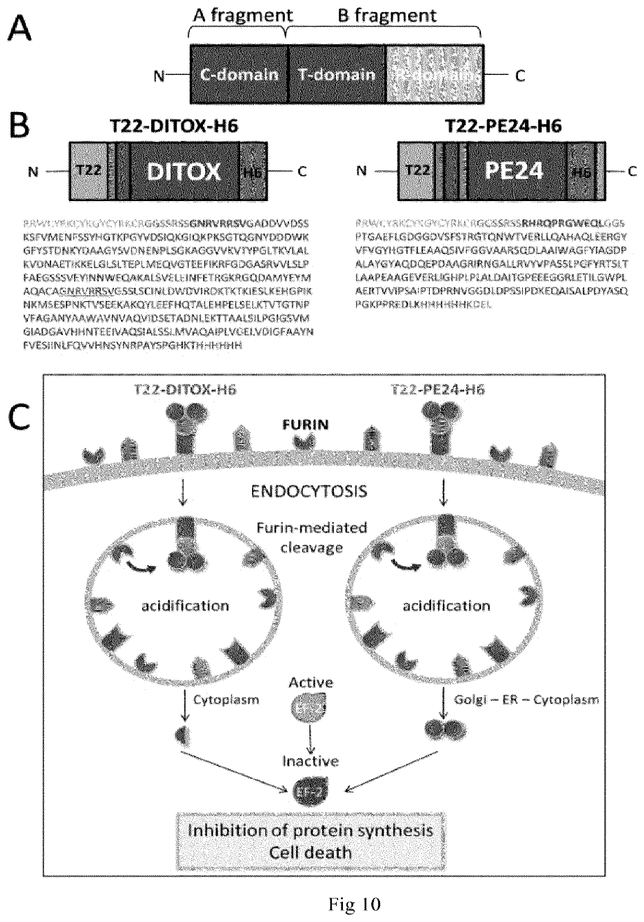

Synthetic genes encoding the self-assembling modular proteins T22-DITOX-H6 and T22-PE24-H6 respectively were designed in-house (FIG. 10A) and provided by Geneart (ThermoFisher).DITOX contains the translocation and catalytic domains of the diphtheria toxin from Corynebacterium diphtheriae. PE24 is based in the de-immunized catalytic domain of Pseudomonas aeruginosa exotoxin A in which point mutations that disrupt B and T cell epitopes have been incorporated. Moreover, it has been added a KDEL sequence in the C-terminus of T22-PE24-H6, which enables the binding to KDEL receptors more efficiently at the Golgi apparatus during subsequent intracellular trafficking. Furin cleavage sites were inserted between the CXCR4 ligand T22 and the functional toxin (FIG. 10A) to release the amino terminal peptide once internalized into target cells. This has been designed so as the natural version of both toxins act with free amino termini, and the recombinant versions proved to be active also show this terminal end in absence of additional peptide segments. Both gene fusions were inserted into the plasmid pET22b, and the recombinant versions of the vector were transformed by heat shock in Escherichia coli Origami B (BL21, OmpT-, Lon-, TrxB-, Gor-, Novagen, Darmstadt, Germany). Transformed cells were grown at 37° C. overnight in LB medium supplemented with 100 μg/ml ampicillin, 12.5 μg/ml tetracycline and 15 μg/ml kanamycin. The encoded proteins were produced at 20° C. overnight upon addition of 0.1 and 1 mM IPTG (isopropyl-β-D-thiogalactopyranoside) for T22-DITOX-H6 and T22-PE24-H6 respectively, when the OD550 of the cell culture reached around 0.5-0.7. Bacterial cells were centrifuged during 15 min (5000 g at 4° C.) and kept at −80° C. until use. Pellets were thaw and resuspended in Wash buffer (20 mM Tris-HCl pH 8.0, 500 mM NaCl, 10 mM imidazole) in presence of protease inhibitors (Complete EDTA-Free, Roche Diagnostics, Indianapolis, IN, USA). Cell disruption was performed by French Press (Thermo FA-078A) at 1200 psi. The lysates were then centrifuged for 45 min (15,000 g at 4° C.), and the soluble fraction was filtered using a pore diameter of 0.2 μm. Proteins were then purified through the His-tag by Immobilized Metal Affinity Chromatography (IMAC) using a HiTrap Chelating HP 1 ml column (GE Healthcare, Piscataway, NJ, USA) with an AKTA purifier FPLC (GE Healthcare). Elution was achieved using a lineal gradient of Elution buffer (20 mM Tris-HCl pH 8.0, 500 mM NaCl and 500 mM imidazole). The eluted fractions were collected, dialyzed against carbonate buffer (166 mM NaCO3H pH 8) and centrifuged for 15 min (15,000 g at 4° C.) to remove insoluble aggregates. The integrity and purity of the proteins was analyzed by mass spectrometry (MALDITOF), SDS-PAGE and Western blotting using anti-His monoclonal antibody (Santa Cruz Biotechnology, Santa Cruz, CA, USA). Protein concentration was determined by Bradford's assay. The nomenclature used for the fusion proteins has been established according to their modular organization.

### Furin Cleavage Design and Detection

To promote the intracellular release of ligand-free toxins of the T22-DITOX-H6 and T22-PE24-H6 fusion proteins, two different furin cleavage sites, naturally acting in the respective toxin precursors to activate translocation, were included in T22-DITOX-H6 and T22-PE24-H6 (FIG. 10A). Efficiency of cleavage in the platform was assessed in T22-DITOX-H6, since the expected fragments should exhibit fully distinguishable molecular masses suitable for quantitative analysis. For that, HeLa cell extracts exposed to 1 μM protein for 24 h were submitted to a Western Blot analysis. After protein incubation, cells were collected, centrifuged, suspended in DPBS and disrupted by sonication. The Western Blot bands were quantified using Image Lab Software version 5.2.1. Two additional modular proteins were also constructed in which these engineered furin cleavage sites were not included, namely T22-DITOX-H6 F- and T22-PE24-H6 F-. Their amino acid sequence exactly matched that of the equivalent constructs T22-DITOX-H6 and T22-PE24-H6 at exception of the boldface dark blue peptide (FIG. 10 A), corresponding to the protease target site. These non-cleavable constructs were used for a comparative analysis of protein cytotoxicity.

### Fluorescence Labelling and Dynamic Light Scattering

Regarding the fluorescence labelling and dynamic light scattering of the T22-DITOX-H6 and T22-PE24-H6 fusion proteins, said fusion proteins were labelled with ATTO 488 (Sigma Aldrich, Buchs, Switzerland) to track their internalization when performing in vitro and in vivo experiments. The conjugation was performed at a molar ratio of 1:2 at room temperature in darkness. The reaction mixture was gently stirred every 15 min during 1 h, centrifuged for 15 min (15,000 g at 4° C.) and dialyzed overnight in the original buffer (166 mM NaCO3H pH 8) to eliminate free ATTO.

Fluorescence of the nanoparticles at 0.1 mg/ml was determined by a Varian Cary Eclipse fluorescence spectrophotometer (Agilent Technologies, Mulgrave, Australia) at 523 nm using an excitation wavelength of 488 nm. For comparative analyses, the intensity of fluorescence was corrected by protein amounts to render specific emission values. Stability of dye conjugation was assessed through the incubation of T22-DITOX-H6* at a final concentration of 0.5 μg/μl in human serum (52257-5ML, Sigma, St Louis, MO, USA) for 48 h at 37° C., with gentle agitation. Then, the sample was dialyzed in 300 ml of carbonate buffer (166 mM NaCO3H, pH 8) for 2 h to remove the free ATTO that might have been released from the nanoparticle. In parallel a positive control was dialyzed containing the same amount of free ATTO. The fluorescence of buffers obtained after the dialysis was measured in the fluorimeter. The volume size distribution of all nanoparticles was determined by dynamic light scattering (DLS) at 633 nm (Zetasizer Nano ZS, Malvern Instruments Limited, Malvern, Worcestershire, UK).

### Ultrastructural Characterization

Size and shape of T22-DITOX-H6 and T22-PE24-H6 nanoparticles at nearly native state were evaluated with a field emission scanning electron microscope (FESEM) Zeiss Merlin (Zeiss, Oberkochen, Germany) operating at 1 kV. Drops of 3 μl of each protein sample were directly deposited on silicon wafers (Ted Pella Inc., Reading, CA, USA) for 1 min, excess blotted with Whatman filter paper number 1 (GE Healthcare, Piscataway, NJ, USA), air dried, and observed without coating with a high resolution in-lens secondary electron detector. For each sample, representative images of different fields were captured at magnifications from 120,000× to 200,000×.

### Cell Culture and Flow Cytometry

CXCR4+ cervical, colorectal and pancreatic cancer cell lines were used to study the performance of the recombinant proteins in vitro (HeLa ATCC-CCL-2, SW1417 ATCC-CCL-238 and Panc-1 ATCC-CCL-1469). HeLa cells were maintained in Eagle's Minimum Essential Medium (Gibco®, Rockville, MD, USA), whereas SW1417 and Panc-1 in Dulbecco's Modified Eagle's Medium (Gibco®). All of them were supplemented with 10% foetal bovine serum (Gibco®) and incubated in a humidified atmosphere at 37° C. and 5% of CO2 (at 10% for SW1417 cells).

In order to monitor protein internalization, HeLa cells were cultured on 24-well plates at 3×10⁴ cells/well for 24 h until reaching 70% confluence. Proteins were incubated for 1 h at different concentrations (100, 500 and 1000 nM) in presence of OptiPRO™ SFM supplemented with L-glutamine. Additionally, specific internalization through CXCR4 receptor was proved adding a specific antagonist, AMD3100, which is expected to inhibit the interaction with T22. This chemical inhibitor was added 1 h prior protein incubation at a ratio of 1:10. Furthermore, kinetics of the internalization was performed at a concentration of 1 μM, after different periods of incubation (0, 20, 30, 60, 120, and 240 min). After protein exposure, cells were detached using 1 mg/ml Trypsin-EDTA (Gibco®) for 15 min at 37° C., a harsh protocol designed to remove externally attached protein (Richard J. P. et al., J. Biol. Chem. 2003, 278: 585-590). The obtained samples were analyzed by a FACS-Canto system (Becton Dickinson, Franklin Lakes, NJ, USA) using a 15 mW air-cooled argon ion laser at 488 nm excitation. Experiments were performed in duplicate.

### Confocal Laser Scanning Microscopy

For confocal microscopy HeLa cells were grown on Mat-Tek plates (MatTek Corporation, Ashland, MA, USA). Upon exposure to the materials cell nuclei were labelled with 5 μg/ml Hoechst 33342 (ThermoFischer, Waltham, MA, USA) and the plasma membrane with 2.5 μg/ml CellMask™ Deep Red (ThermoFischer) for 10 min at room temperature. Cells were then washed in PBS buffer (Sigma-Aldrich, Steinheim, Germany). The confocal images of the HeLa cells were collected on an inverted TCS SP5 Leica Spectral confocal microscope (Leica Microsystems, Wetzlar, Germany) using 63× (1.4 NA) oil immersion objective lenses. Excitation was reached via a 405 nm blue diode laser (nucleic acids), 488 nm line of an argon ion laser (nanoparticles) and 633 nm line of a HeNe laser (Cell membrane). Optimized emission detection bandwidths were configured to avoid inter-channel crosstalk and multitrack sequential acquisition setting were used. The confocal pinhole was set to 1 Airy unit and z-stacks acquisition intervals were selected to satisfy Nyquist_sampling criteria. Three-dimensional images were processed using the Surpass Module in Imaris X64 v.7.2.1. software (Bitplane, Zurich, Switzerland).

### Cell Viability Assays

The CellTiter-Glo® Luminescent Cell Viability Assay (Promega, Madison, WI, USA) was used to determine the cytotoxicity of T22-DITOX-H6, T22-PE24-H6, T22-DITOX-H6 F- and T22-PE24-H6 F- nanoparticles on HeLa, SW1417 CXCR4+ or SW1417 CXCR4− cell lines. Cells were cultured in opaque-walled 96-well plates at 3500 or 6000 cells/well during 24 h at 37° C. until reaching 70% confluence. All protein incubations were performed in the corresponding medium according to the cell line used. Inhibition of cell death was analyzed by adding AMD3100, a chemical antagonist of CXCR4, at a ratio of 1:10, 1 h prior to protein incubation. T22-GFP-H6, a non-functional T22-bearing protein, was also used as a competitor of T22-empowered toxins at a final concentration of 2 μM. After protein incubation, a single reagent provided by the manufacturer was added to cultured cells, which prompted lysis and generated a luminescent signal proportional to the amount of ATP present in the sample. The ATP generated is directly related to the quantity of living cells that remain in the well. Then, plates were measured in a conventional luminometer, Victor3 (Perkin Elmer, Waltham, MA, USA). Viability of Panc-1 cells, that overexpress luciferase, was determined with an alternative non fluorescence kit (EZ4U) under the same experimental conditions. The cell viability experiments were performed in triplicate.

Biodistribution, Pharmacokinetics and Apoptotic Induction Analyses in CXCR4+ Colorectal Cancer Mouse Model after Single Dose Administration of Nanoparticles

All in vivo experiments were approved by the institutional animal Ethics Committee of Hospital Sant Pau. We used 5 week-old female Swiss Nu/Nu mice, weighing 18-20 g (Charles River, L'Abresle, France), maintained in specific pathogen-free conditions. To generate the subcutaneous

(SC) mouse model, we implanted subcutaneously 10 mg of the patient-derived M5 colorectal (CCR) tumor tissue from donor animals in the mouse subcutis. At day 15, when tumors reached approximately 500 mm3, mice received 50 μg single i.v. bolus of T22-DITOXH6* (n=3) or 300 μg single i.v. bolus of T22-PE24-H6* (n=3) in NaCO3H, pH=8 buffer. Control animals received the same buffer (n=3) or 0.25 μg of free ATTO 488 (n=2). At 5, 24 and 48 h mice were euthanized and subcutaneous tumors and organs (brain, lung, liver, kidney and heart) were collected. Biodistribution of ATTO-labelled nanoparticles in tumor and non-tumor organs was determined by measuring the emitted fluorescence in ex vivo tissue sections (3 mm thick) using the IVIS® Spectrum (Perkin Elmer, Santa Clara, CA, USA) platform. The fluorescent signal (FLI), which correlates to the amount of administered protein accumulated in each tissue, was first digitalized,
displayed as a pseudocolor overlay, and expressed as radiant efficiency [(p/s/cm2/sr)/μW/cm2]. The FLI values were calculated subtracting FLI signal from experimental mice by FLI auto-fluorescence of control mice. Samples were first fixed with 4% formaldehyde in PBS for 24 h to be embedded in paraffin for histopathological evaluation and apoptotic index analyses. Pharmacokinetic analyses were performed after a 300 μg single i.v. bolus administration of T22-PE24-H6* in 12 Swiss nude mice, or after a 50 μg single bolus administration of T22-DITOX-H6* also in 12 animals.

Three mice per each time point, at 0, 1, 2, 5, 24 and 48 h after the administration were sacrificed and approximately 1 ml of blood EDTA anticoagulated collection tubes were obtained. The exact volume of plasma obtained and the fluorescent emission at each time point were measured, and the concentration of nanoparticle as referred to the fluorescence emitted and concentration of the administered dose calculated. Apoptotic induction analyses were performed in 4 m sections of tumors and normal organs (liver, lung, spleen, heart, kidney and brain) stained with hematoxylin and eosin (H&E), which were histopathologically analyzed by two independent observers. Apoptotic induction was evaluated by both, the presence of cell death bodies in H&E stained and Hoechst stained tumor slices. Triton X-100 (0.5%) permeabilized sections were then stained with Hoechst 33258 (Sigma-Aldrich) diluted, 1:5000 in PBS, for 1 h, rinsed with water, mounted and analyzed under fluorescence microscope (λex=334 nm/λem=465 nm). The number of apoptotic cell bodies was quantified by recording the number of condensed and/or defragmented nuclei per 10 high-power fields (magnification 400×), in blinded samples evaluated by two independent researchers, using Cell∧B s.

Antitumor Effect in a CXCR4+ CRC Model after Nanoparticle Repeated Dose Administration

To generate the CXCR4+ colorectal xenograft mouse models, we used the patient-derived M5 colorectal tumor tissue. Ten mg fragments obtained from donor animals were implanted in the subcutis of Swiss nu/nu mice to generate subcutaneous (SC) tumors as described above (n=9). Once tumors reached approximately 120 mm3, mice were randomized in Control, T22-PE24-H6 and T22-DITOX-H6 groups and received intravenous doses of T22-PE24-H6 or T22-DITOX-H6, both at a repeated dose regime of 10 μg, 3 times a week, per 8 doses. The control group received buffer using the same administration schedule. Mouse body weight was registered over the experimental period 3 times a week. Seventeen days after the initiation of nanoparticle administration, mice were euthanized and the subcutaneous tumors were taken to measure their final tumor volume and to count the number of apoptotic figures in 5 high-power fields (magnification 400×), of H&E stained tumor sections as described above.

### Statistical Analysis

The specificity of nanoparticle-promoted cell death and the pairwise data comparisons were checked with a one-way ANOVA and Tukey's tests, respectively. Pairwise divergences of internalization and cell death were evaluated using Student's t-tests, whereas Mann-Whitney U tests were used to pairwise comparisons of the number of apoptotic bodies. Differences between groups were considered significant at p<0.05 and differences between relevant data are indicated by letters or as Y for 0.01<p<0.05 and § for p<0.01 in the Figures. All statistical analyses were performed using SPSS version 11.0 package (IBM, NY, USA), and values were expressed as mean±standard error of the mean (SEM).

Materials and Methods Regarding Protein Nanoparticles Based on Recombinant Ricin (mRTA)

### Genetic Design and Protein Production

The recombinant protein T22-mRTA-H6 (FIG. 17 A) was designed to include the highly specific CXCR4 ligand T22 at the amino terminus followed by a mutated version of the ricin A chain, and a hexahistidine tail at the carboxy terminus. The mutation N132A was introduced to suppress the vascular leak syndrome in potential future in vivo administrations, keeping the cytotoxic activity. In addition, a furin cleave site was also incorporated to allow the release of the accessory N-terminal region in the endosome and the intracellular activity of ricin in a quasi-native sequence format. A KDEL motif was also incorporated to favor endosomal escape. The plasmid construct pET22b-T22-mRTA-H6, encoding the protein under the control of the bacteriophage T7 promoter, was generated by GeneArt and transformed into Escherichia coli Origami B cells.

### Production and Purification of Soluble Protein

Recombinant bacteria were cultured in lysogeny broth (LB) medium with 100 μg/ml ampicillin, 15 μg/ml kanamycin and 12.5 μg/ml of tetracycline, at 37° C. and 250 rpm. The recombinant gene expression was induced by adding 0.1 mM isopropyl-β-thiogalactopyronaside (IPTG) when the OD of the culture reached a value between 0.5 and 0.7. Cultures were subsequently incubated overnight at 20° C. and 250 rpm. Cells were harvested and centrifuged (5,000 g, 15 min, 4° C.). The cell pellet was resuspended in Wash Buffer (51 mM sodium phosphate buffer, pH=8, 158.6 mM trehalose dihydrate, 0.01% Polysorbate-20, 15 mM imidazole, 300 mM NaCl) in presence of protease inhibitor cocktail Complete EDTA-Free (Roche). Bacterial cells were sonicated twice at 10% amplitude and once at 15% of amplitude for 10 min each round, centrifuged (15,000 g, 45 min, 4° C.) and soluble fraction purified by affinity chromatography with a HiTrap Chelating HP column in an AKTA purifier FPLC, (GE Healthcare). After the samples were filtered (0.22 m) and injected into the column, the fractions to be collected were eluted at approximately 30% Elution Buffer (51 mM sodium phosphate, pH=8, 158.6 mM trehalose dihydrate, 0.01% Polysorbate-20, 500 mM imidazole, 300 mM NaCl). The buffer exchange was done in Centricon Centrifugal Tubes Ultracel 10,000 NMWL. T22-mRTA-H6 was found to be highly stable in 51 mM sodium phosphate pH=6.2, 60 mg/ml α-trehalose dehydrate, 0.01% polysorbate-20. Protein purity was analyzed by SDS electrophoresis on TGX Stain-Free gels (Bio-Rad), followed by Western blotting using an anti-His monoclonal antibody (Santa Cruz Biotechnology). Sodium dodecyl sulfate polyacrylamide gel electrophoresis (SDS-PAGE) on TGX Stain-Free Gels (Bio-Rad) was conducted to analyze the protein. Samples were diluted in denaturing buffer (0.53 M Tris Base, 5.52 M glycerol, 0.27 M SDS, 2.84 M β-mercaptoethanol, 7.99 M urea) at a 3:1 molar ratio, boiled at 96° C. for 10 min and loaded into the gels lanes. For the Western Blot, an anti-His monoclonal antibody was used (Santa Cruz Biotechnology) followed by a goat anti mouse IgG (H+L)-HRP secondary antibody (Ref: 170-6516) conjugate (Bio-Rad, Ref: 170-6516). Images were observed using ChemiDoc Touch Imaging System. Protein production has been partially performed by the ICTS “NANBIOSIS”, more specifically by the Protein Production Platform of CIBER-BBN/IBB (http://www.nanbiosis.es/unit/ul-protein-production-platform-ppp/).

### Quantitative Protein Analysis

Protein purity was analyzed by sodium dodecyl sulphate polyacrylamide gel electrophoresis (SDS-PAGE) on a Chemi Doc Touch Imaging System (Bio-Rad). Briefly, both soluble and insoluble samples were mixed with in denaturing buffer (0.53M Tris Base, 5.52 M glycerol, 0.27 M sodium dodecylsulphate (SDS), 2.84 M β-mercaptoethanol, 7.99 M urea) at a ratio 3:1, boiled for 5 or 45 min, respectively, and loaded onto the gels. For the Western Blot, an anti-His monoclonal antibody was used (Santa Cruz Biotechnology) followed by a goat anti mouse IgG (H+L)-HRP secondary antibody conjugate (Bio-Rad). Gels were scanned at high resolution and bands were quantified with Quantity One Software (Bio-Rad) using a known protein standard of soluble recombinant T22-mRTA-H6.

### Quantitative and Qualitative Analyses of Soluble Protein

Protein molecular weight was verified by mass spectrometry (MALDI-TOF), and concentration determined by Bradford Assay (Dye Reagent Concentrate Bio-Rad kit). Volume size distribution of protein nanoparticles was determined by Dynamic Light Scattering (DLS). For that, a 50 μl aliquot (stored at −80° C.) was thawed and the volume size distribution of nanoparticles was immediately determined at 633 nm (Zetasizer Nano ZS, Malvern Instruments Limited). Far-UV circular dichroism (CD) was determined at 25° C. in a Jasco J-715 spectropolarimeter to assess the secondary structure of T22-mRTA-H6, which was dissolved at 0.35 mg/ml in 166 mM sodium bicarbonate buffer, pH 8. The CD spectra were obtained in a 1 mm path-length cuvette over a wavelength range of 190-260 nm, at a scan rate of 50 nm/min, a response of 1 s and a band-with of 1 nm. Six scans were accumulated. The magnitude of secondary structure was analyzed using the JASCO spectramanager analysis software. To investigate potential intermolecular β-sheet structure in the protein nanoparticles, conventional methods for Thioflavin T (ThT) staining weere adapted.

Briefly, protein aliquots (10 μl) were added to 90 μl of 50 μM (Sigma Aldrich) in phosphate buffered saline (PBS), pH 7,4 and stirred for 1 min. The final protein concentration was 0.17 mg/ml. ThT was excited at 450 nm and the fluorescence emission spectra was recorded in the range of 460 to 565 nm with a Varian Cary Eclipse spectrofluorimeter. The cross-β-sheet structure was monitored by the enhancement of the free dye fluorescence emission.

### Cell Culture and Determination of Cell Viability and Apoptosis

HeLa cells (ATCC-CCL-2) were cultured at 37° C. in a 5% CO2 humidified atmosphere in MEM-Alpha media supplemented with 10% fetal calf serum (Gibco Thermo Fisher Scientific (TFS)). They were seeded in an opaque 96-well plate (3×104 cells/well) for 24 h. When insoluble T22-mRTA-H6 was assayed, the media was supplemented with 2% penicillin, 10,000 U/ml streptomycin (Gibco, TFS). The next day soluble T22-mRTA-H6 was added and cells were exposed for 24, 48 and 72 h). Cells were also exposed to insoluble protein version during 24, 48, 72, 96, 120, and 144 h. Cell viability was determined by CellTiterGlo Luminescent Cell Viability Assay (Promega) in a Multilabel Plater Reader Victor3 (Perkin Elmer). For the CXCR4 specificity assay, the CXCR4 antagonist AMD3100 was added at 10:1 molar ratio 1 h before the incorporation of the protein. Antagonist and protein were incubated in a final volume of 10 μl that were mixed with 90 μl of culture media. All soluble protein experiments were done in triplicate and insoluble protein with six replicates. On the other hand, the AML cell lines THP1 (ACC-16) and MV411 (ACC-102), as well as 3T3 mouse fibroblasts (ACC-173), were purchased from DSMZ (Leibniz Institute DSMZGerman Collection of Microorganisms and Cell Cultures, Braunschweig, Germany). THP1 was cultured in RPMI-1640 medium supplemented with 10% FBS, 10 mmol/l Lglutamine 100 U/ml penicillin, 10 mg/ml streptomycin and 0.45 μg/ml fungizone. (Gibco, TFS). 3T3 cells were cultured with DMEM medium adding the same supplements. Cells were kept at 37° C. in a humidified atmosphere of 5% CO2. Cell viability assays with these cell lines were performed using the XTT Cell Viability Kit II (Roche Diagnostics) and absorbance was read in a spectrophotometer at 490 nm (BMG Labtech). The effect of the caspase inhibitor zVAD-fmk was evaluated pretreating for 1 hour cells seeded on 96-well plates (at 100 μM zVAD-fmk) and then exposing them to 100 nM T22-mRTA-H6 for 48 hours. The antitumor drug Ara-C(Cytosine β-D-arabinofuranoside hydrochloride) was purchased from Sigma Aldrich. To allow the follow-up of AML in mice, THP1 AML cell line was transfected with a plasmid encoding the luciferase gene that confers bioluminescence that can be noninvasively imaged (BLI) to the cells. Briefly, THP1 cells were harvested in 24-well plates, treated with 0.5 μg of DNA plasmid and mixed with Lipofectamine LTX and PLUS reagents (A12621, Invitrogen, TFS) in Opti-MEM Reduced Serum Medium (Gibco, TFS) according to the manufacturer's instructions. 48 hours later BLI levels were tested incubating cells with luciferin in an IVIS Spectrum In Vivo Imaging System (PerkinElmer, Waltham, MA, USA). Finally, transfected cells were selected with 1.5 mg/mL geneticin (G418 Sulfate, Gibco, TFS) and BLI was analyzed periodically to check the preservation of the plasmid in cells, called THP1-Luci cells. Internalization of T22-GFP-H6 in 3T3, MV411, THP1 and HeLa was determined by Fluorescence-activated cell sorting (FACS Calibur, BD).

Cells were exposed for 1 hour to the T22-GFP-H6 concentrations at 100 nM. Then, cells were washed with PBS and trypsinized (1 mg/ml trypsin, Life Technologies) in order to remove nonspecific binding of nanoparticles to the cell membrane. Finally, levels of intracellular GFP fluorescence were quantified by flow cytometry. Mean fluorescence intensity ratios are given as mean fluorescence intensity of the treated samples divided by the mean fluorescence intensity of the vehicles.

To evaluate cell apoptosis, nuclear staining was performed with the Hoescht 3342 dye (Sigma-Aldrich) in HeL1a cells exposed to 100 nM T22-mRTA-H6 or buffer for different times. Once the incubation was finished, the media was collected and centrifuged to obtain the suspended cells. They were rinsed with PBS and centrifuged again. The adhered cells were trypsinized and pulled together with those previously obtained. These cells were fixed (3.7% p-formaldehyde in PBS, pH 7.4) for 10 min at −20° C., washed with PBS and resuspended in 10 μl of PBS. Finally, cells were mounted on a slide with ProLong™ Gold Antifade Mountant with DAPI and observed for the appearance of the nuclei under a fluorescence microscope. In addition, externalizad phosphatidylserine protein-exposed cells was detected by Annexin Annexin V Detection Kit (APC, eBioscience) while dead cells were spotted with propidium iodide (PI), according to supplier instructions. Cell internalization was monitored using ATTO-labelled protein as described elsewhere.

### Determination of ROS Levels and Mitochondrial Damage

On the other hand, levels of cellular ROS were measured with the Cellular ROS Detection Assay Kit (Abcam). In brief, HeLa cells were exposed to 100 nM T22-mRTA-H6 (15 or 24 hours) or buffer. Then, cells were washed and incubated with ROS Detection Solution for 1 hour at 37° C., in the dark, adding 100 μM Pyocyanin (1 hour) to the positive controls. Afterwards, levels of fluorescence were read with a microplate reader (BMG Labtech) at Ex=488 nm and Em=520 nm. Values were expressed as relative fluorescence units after subtracting the background fluorescence of blanks. Finally, to measure mitochondrial membrane potential (Δψm), we used a mitochondrial potential detection kit (BD MitoScreen, BD Biosciences) according to manufacturer's instructions. Labelled cells were analyzed by flow cytometry and the data were expressed as percentage of cells containing depolarized mitochondria (loss of JC-1 red fluorescence).

### Flow Cytometry

CXCR4 membrane expression was determined by Fluorescence-activated cell sorting (FACS Calibur, BD). Cells were washed with PBS 0.5% BSA and incubated either with PE-Cy5 mouse anti-CXCR4 monoclonal antibody (BD Biosciences) or PE-Cy5 Mouse IdG2a isotype (BD Biosciences) as control. Results of fluorescence emission were analyzed with software Cell Quest Pro and expressed as the ratio between the mean fluorescence intensity of each sample and the isotype values.

### Electron Microscopy

The ultrastructure of soluble (in form of nanoparticles) and insoluble (in form of IBs) T22-mRTA-H6 was observed by field emission scanning electron microscopy (FESEM). Insoluble protein was resuspended in PBS and sonicated at 10% amplitude 0.5 s ON/OFF for 1 min. Drops of 10 μL of either soluble protein in storage buffer or insoluble protein in PBS were deposited during 1 min on silicon wafers (Ted Pella), excess of liquid eliminated, and air dried. Samples without coating were observed with an in-lens detector in a FESEM Zeiss Merlin (Zeiss) operating at 1 kV. Representative images were obtained at a wide range of magnifications (from 100,000× to 450,000×).

### Antineoplastic Effect in a Disseminated Acute Myeloid Leukemia (AML) Mouse Model

NSG (NOD-scid IL2Rgammanull) female mice (5 weeks old) were obtained from Charles River Laboratories (Wilmington, MA, USA) and housed in microisolator units with sterile food and water ad libitum. After 1 week in quarantine, NSG mice were intravenously (IV) injected with luciferase-transfected THP1 cells (THP1-Luci; 1×106 cells/200 L) and divided randomly into three different experimental groups. One group (VEHICLE; n=3) was IV injected with NaCO3H pH=8 buffer, a second group (T22mRTA; n=1) was administered with 10 μg of T22-mRTA-H6. Both groups were injected with a daily dose for a total of 10 doses. A third group (IB-T22mRTA; n=2) was subcutaneously (SC) injected once with 1 mg of T22-mRTA-H6 IBs. These treatments started 2 days after the IV injection of THP1-Luci cells in mice, which generated the disseminated AML model. Evolution of AML dissemination was monitored in IVIS Spectrum three times per week until the day of the euthanasia. Weight of the animals was measured the same day of BLI analysis. All mice were euthanized the day that the first of them presented relevant signs of disease such as 10% weight loss or lack of mobility. Animals were intraperitoneally injected with luciferin, and after 5 min mice were killed by cervical dislocation. Tissues were excised and the BLI levels of the organs ex vivo analyzed. After that, they were preserved in formaldehyde 3.7% and paraffin embedded for further immunohistochemistry analyses. The analysis and detection of BLI was performed using radiance photons in Living Image 4.4 Software both in in vivo and ex vivo studies. All procedures were conducted in accordance with the guidelines approved by the institutional animal Ethics Committee of Hospital Sant Pau.

### Histopathology and Immunohistochemical Staining

Sections of paraffin-embedded samples of infiltrated (liver, spleen, hindlimbs and backbone) and normal (lung, heart and kidney) organs were hematoxylin and eosin (H&E) stained and the presence of toxicity was analyzed. Moreover, in order to detect AML cells in infiltrated tissues, immunohistochemical analysis with anti-human CD45 antibody (DAKO) was done in paraffin-embedded tissue samples. Staining was performed in a Dako Autostainer Link 48, following the manufacturer's instructions. Two independent observers evaluated all samples, using an Olympus BX51 microscope (Olympus). Images were acquired using an Olympus DP72 digital camera and processed with CellD Imaging 3.3 software (Olympus).

### Statistical Analysis

Quantitative data are expressed as mean±standard error (SE). Previously to perform statistical analyses, all variables were tested for normality and homogeneity of variances employing the Shapiro-Wilk and the Levene test, respectively. Comparisons of soluble protein cytotoxicity effects and competition assays were made with Tukey's test. Meanwhile protein cytotoxicity assays were assessed by Mann-Whitney U tests. Significance was accepted at p<0.05.

### Example 1: Design and Characterization of the T22-BAK-GFP-H6 Fusion Protein and Nanoparticles

The inventors designed a fusion protein that comprised the cationic peptide T22, a potent CXCR4-ligand to the BAK BH3 domain, for the construction of a BAK-based building block. GFP was incorporated to the fusion platform to conveniently monitor the localization of the material and to explore the potential use of the material in diagnosis as well as in therapy (or for theragnosis). A schematic representation of the fusion protein can be seen in FIG. 1A.

The chimeric protein was biofabricated in Escherichia coli and purified by conventional procedures (as specified in the materials and methods section) in form of a unique and stable molecular species with the expected mass (FIG. 1B). As expected, the protein spontaneously assembled into discrete, monodisperse materials of about 13.5 nm in diameter, which upon treatment with SDS disassembled into building blocks of >7 nm (FIG. 1C). T22-BAK-GFP-H6 monomers were slightly larger than BAK-GFP-H6 protein (<7 nm), that remained unassembled because of the absence of the cationic T22. Disassembling was not observed upon 5 h incubation in Optipro complex culture medium (not shown), indicative of stability of nanoparticles in complex physiological media.

Also, T22-BAK-GFP-H6 nanoparticles were fluorescent, exhibiting a specific green fluorescence emission of 306.7±7.8 units/g, appropriate for quantitative imaging. High resolution scanning electron microscopy revealed these materials as planar objects with regular morphometry (FIG. 1D).

### Example 2: Functional Analysis of the T22-BAK-GFP-H6 Fusion Protein Nanoparticles

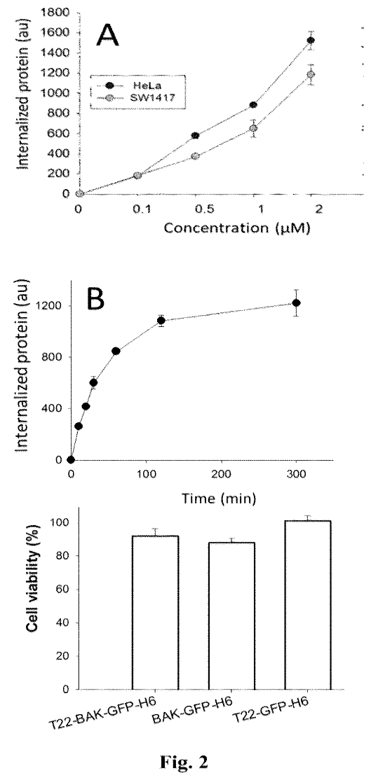

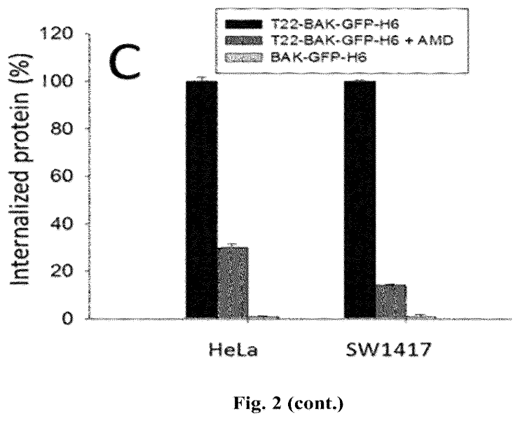

Regarding functional analyses, the inventors first determined the ability of protein nanoparticles to bind and penetrate, in a receptor-dependent way, CXCR4+ cells. Indeed, the assembled T22-BAK-GFP-H6 protein efficiently penetrated CXCR4+ HeLa and SW1417 cells (FIG. 2A). The kinetics of accumulation was compatible with receptor-mediated endocytosis (FIG. 2B), while the uptake was CXCR4-dependent, as the inhibitor of T22-CXCR4 interaction, AMD3100, [Unzueta, U. et al., 2012. Int. J. Nanomedicine. 7:4533-44.] dramatically reduced the intracellular fluorescence in both cell lines upon exposure.

The control T22-devoid construct failed to enter cells (FIG. 2C). The efficient penetration of T22-BAK-GFP-H6 was confirmed by the generic occurrence of fluorescence in most exposed cells (FIG. 2D, and by the intracellular accumulation of the material in the perinuclear region (FIG. 2E). T22-BAK-GFP-H6 was intrinsically non-toxic, as CXCR4− cell viability remained unaltered after prolonged exposure (FIG. 2B, inset).

### Example 3: In Vivo Accumulation and Distribution of the T22-BAK-GFP-H6 Fusion Protein Nanoparticles

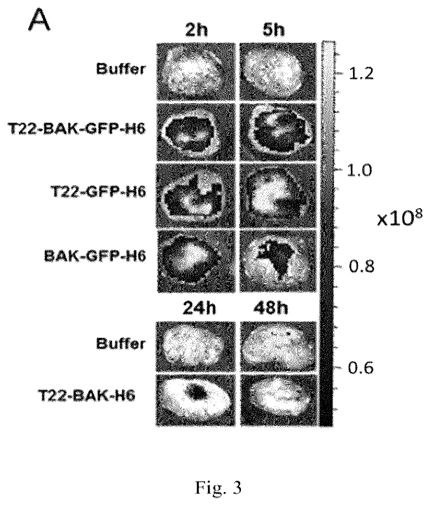

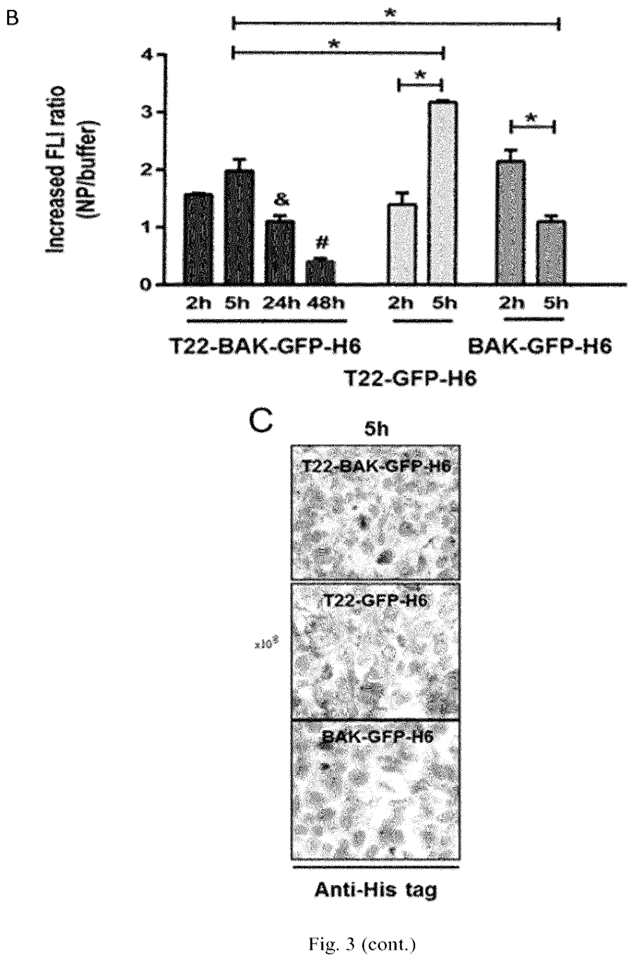

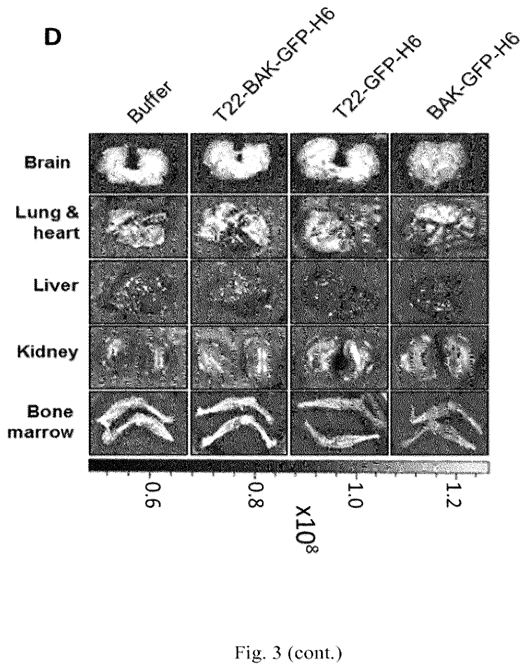

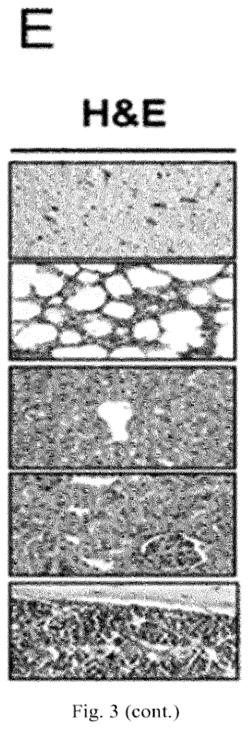

Given the high CXCR4-linked cell penetrability of T22-BAK-GFP-H6 nanoparticles the inventors tested the new material in a mouse model of CXCR4+ colorectal cancer, regarding biodistribution and capacity of the material to induce selective apoptosis in tumor tissues. The systemic administration of T22-BAK-GFP-H6 nanoparticles through the tail vein resulted in a transient accumulation of the material in the tumors, peaking at 5 h as determined by ex vivo fluorescence images and by IHC (FIG. 3A-3C).

Other relevant organs such as kidney showed only residual fluorescence emission levels (FIG. 3D), confirming not only the desired localization of the materials but also the absence of significant renal filtration, aggregation in lung or detectable toxicity in the time-course (FIGS. 3D, 3E). In particular, the absence of protein in kidney was indicative of a high stability of the oligomers, as monomeric or disassembled proteins, even targeted to specific tumor markers, accumulate in kidney [Cespedes, M. V. et al. 2014. ACS Nano, 8:4166-76].

At 24 but not at 48 h, the tumor still showed detectable fluorescence (TABLE 1), indicating prolonged permanence of nanoparticles in the target organ.

TABLE 1
Table 1: Quantitation of GFP fluorescence signal in brain, lung and heart,
liver, kidney and bone marrow tissues expressed as Fluorescent ratio. This was
calculated by dividing the fluorescence of each organ in protein-treated mice
by the autofluorescence measured in buffer-treated mice of the respective
organ of the time-course experiment.
Increased FLI ratio (NP/buffer)
Data expressed as mean ± SE.

| | Groups | | | | |
|---|---|---|---|---|---|
| | T22-BAK-GFP-H6 | | | | |
| Organs | (5 h) | (24 h) | (48 h) | T22-GFP-H6 | BAK-GFP-H6 |
| Brain | 1.21 +/− 0.07 | 0.99 +/− 0.10 | 1.14 +/− 0.08 | 1.26 +/− 0.06 | 1.18 +/− 0.10 |
| Lung & | 1.20 +/− 0.08 | 1.18 +/− 0.02 | 1.25 +/− 0.21 | 1.27 +/− 0.01 | 1.24 +/− 0.14 |
| heart | | | | | |
| Liver | 1.04 +/− 0.01 | 1.11 +/− 0.03 | 1.07 +/− 0.02 | 0.90 +/− 0.04 | 0.96 +/− 0.02 |
| Kidney | 1.07 +/− 0.20 | 1.15 +/− 0.15 | 1.08 +/− 0.14 | 1.36 +/− 0.2 | 1.76 +/− 0.40 |
| Bone | 1.05 +/− 0.02 | 1.19 +/− 0.04 | 0.94 +/− 0.02 | 0.99 +/− 0.03 | 1.03 +/− 0.01 |
| marrow | | | | | |

### Example 4: Effects of the T22-BAK-GFP-H6 Fusion Protein Nanoparticles on Apoptosis and Cell Cycle

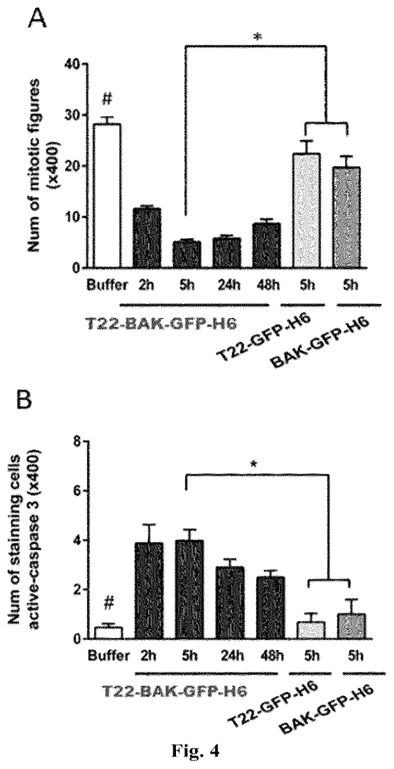

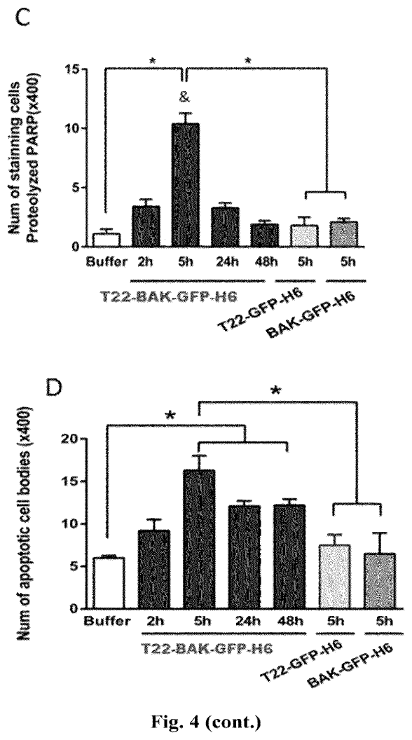

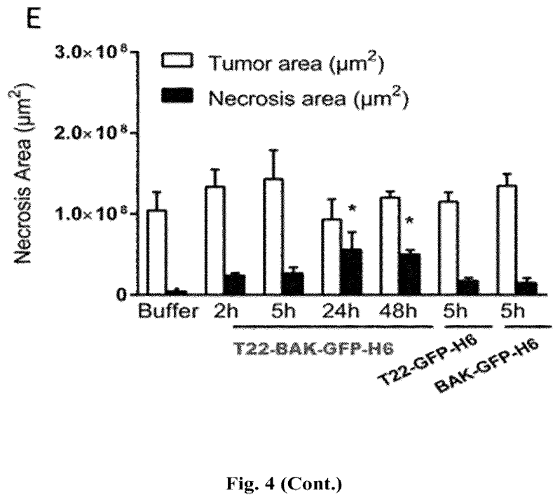

As compared to the parental T22-GFP-H6 or the untargeted BAK-GFP-H6 protein, T22-BAK-GFP-H6 induced a significantly decrease of mitotic figures (FIG. 4A). This was associated with caspase-3 activation, proteolysis of PARP, occurrence of apoptotic bodies and increased necrotic areas in tumor tissues shortly (2 h) after the administration of the material in mice (FIGS. 4B-4F). Tumor cell apoptosis peaked at 5 h and it was maintained for at least 48 h (FIG. 4A).

In contrast, the non-targeted BAK-GFP-H6 protein yielded only a negligible level of caspase-3 activation or apoptosis in tumors, since it did not differ from the background in buffer-treated tumors (FIG. 4F). Histological alterations were not observed in any of the explored non-target organs (FIG. 3E). These observations proved not only the molecular availability of the BAK BH3domain when delivered as regular nanoparticles but, as envisaged, that T22-BAK-GFP-H6 nanoparticles exhibited intrinsic biological activity.

### Example 5: Physical and Biological Characterization of the T22-PUMA-GFP-H6 and T22-GW-H1-GFP-H6 Fusion Proteins' Nanoparticles

At this stage the inventors delved deeper into the alternatives such a platform based on therapeutic protein-only nanoparticles would have. Hence, the inventors tested the formation of functional nanoscale materials based on the p53-upregulated modulator of apoptosis PUMA [Zhang, Y et al. 2009. Mol Biol Cell., 20:3077-87] and the antimicrobial peptide GWH1 [Chen, Y-L. S. et al. 2012. Peptides, 36:257-65], both also inducing apoptosis upon internalization in cancer cells. Under the same modular scheme than T22-BAK-GFP-H6, T22-PUMA-GFP-H6 (FIG. 5A) and T22-GWH1-GFP-H6 (FIG. 5B) form nanoparticles of 20 and 24 nm respectively, that as in three BAK-based construct retain the GFP fluorescence (not shown). When administered in vivo, both nanoparticles accumulate in tumor (FIGS. 5C-5D), with a minor occurrence of T22-GWH1-GFP-H6 in kidney. Both type of nanoparticles significantly reduced the mitotic rates and even with some variability, the rug materials tended to induce cell death and promote selective necrosis in tumoral tissues, this effect being significant in the case of the PUMA-based material (FIGS. 5E-5F).

### Example 6: Characterization of the H6-GFP-R9 and H6-R9-GFP Fusion Proteins

The inventors designed fusions protein comprising, in this order, an hexahistdine region, GFP and a polyarginine sequence (the H6-GFP-R9 fusion protein) and an hexahistidine region, a polyarginine sequence and GFP (the H6-R9-GFP fusion protein).

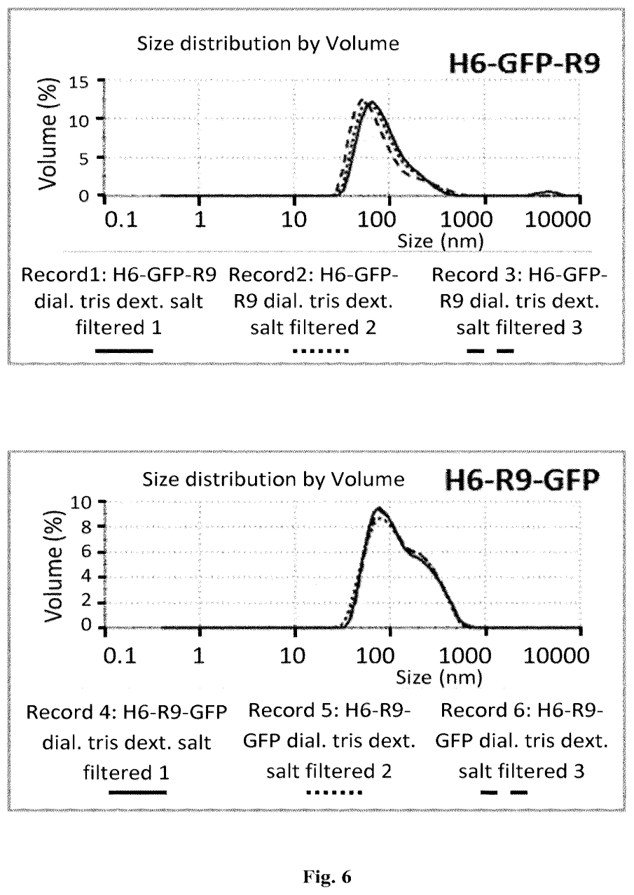

The chimeric proteins were biofabricated in Escherichia coli and purified by conventional procedures (as specified in the materials and methods section) in form of a unique and stable molecular species with the expected mass. The protein spontaneously assembled into discrete, monodisperse materials of about 40-70 nm in diameter in the case of the H6-GFP-R9 fusion protein and of 60-90 nm in diameter in the case of the H6-R9-GFP fusion protein (FIG. 6).

### Example 7: Characterization of GWH1-Based Protein Nanoparticles

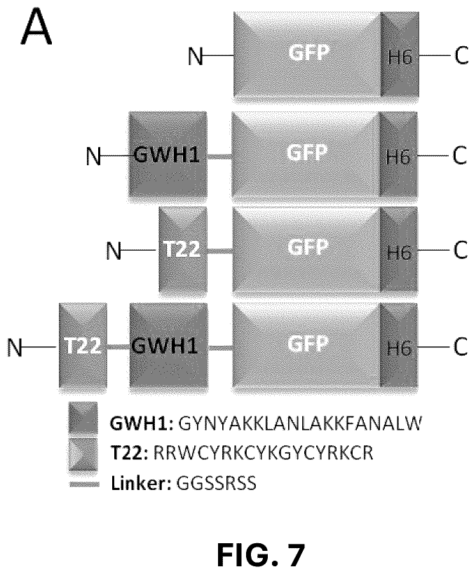

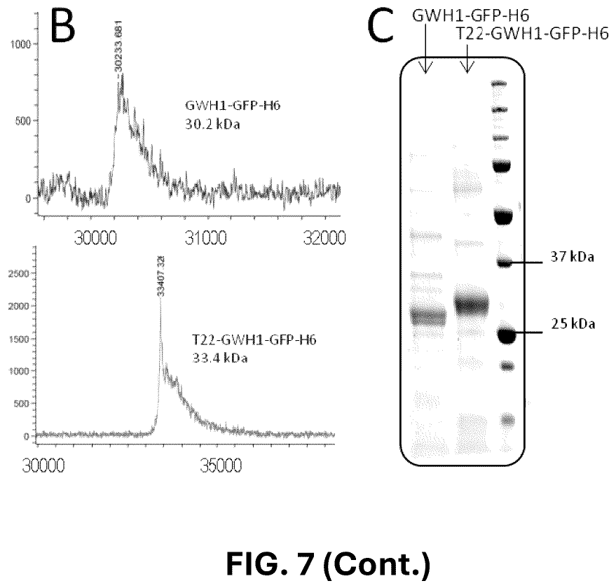

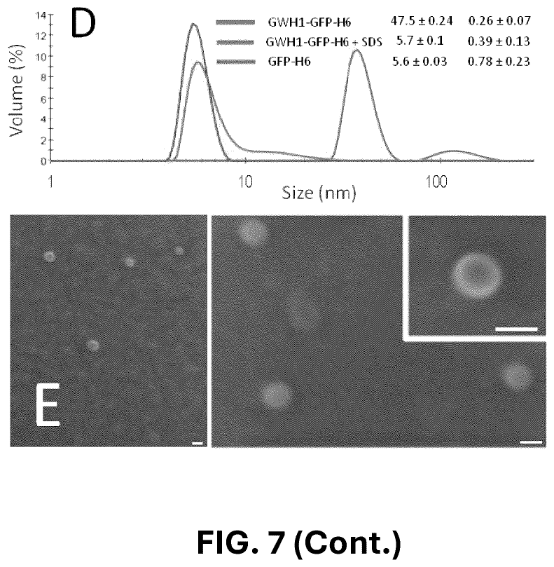

GWH1-GFP-H6 (FIG. 1A) was successfully produced in recombinant E. coli without visible signs of toxicity. Its purification in a single-step by His affinity chromatography rendered a protein species with the expected molecular mass of 30.2 kDa (FIG. 7 B, C). Since the GWH1 peptide is highly cationic and the combination of end terminal cationic peptides plus polyhistidines promotes protein self-assembling, we tested the potential of this protein to form oligomers. Indeed, the spontaneous formation of nanoparticles was detected by DLS in pure preparations of the protein (FIG. 7 D), indicative of the good performance of GWH1 as a nanoscale architectonic tag. Those nanoparticles, peaking at 47 nm, were fully disassembled by 0.1% SDS, rendering building blocks of 5 nm, that matched the size of the parental unassembled GFP-H6 (FIG. 7D). The formation of regular GWH1-GFP-H6 nanoparticles was fully assessed by FESEM (FIG. 1E).

### Example 8: Antibacterial Activities of GWH1-Based Protein Nanoparticles

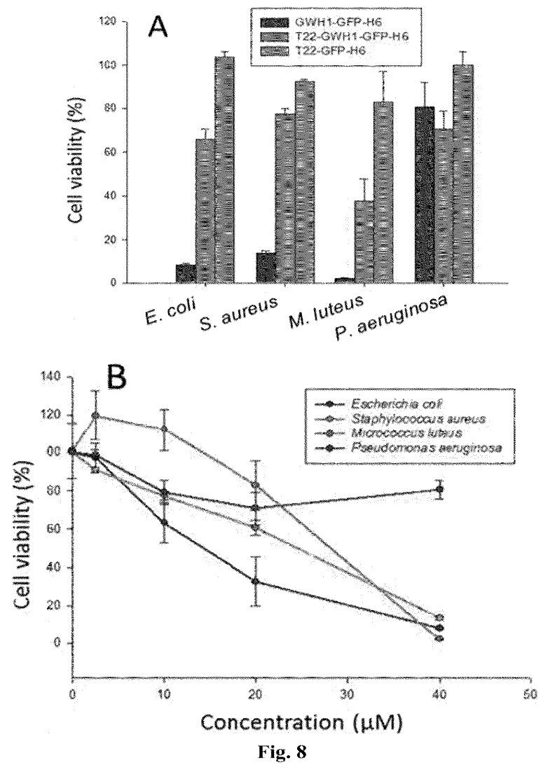

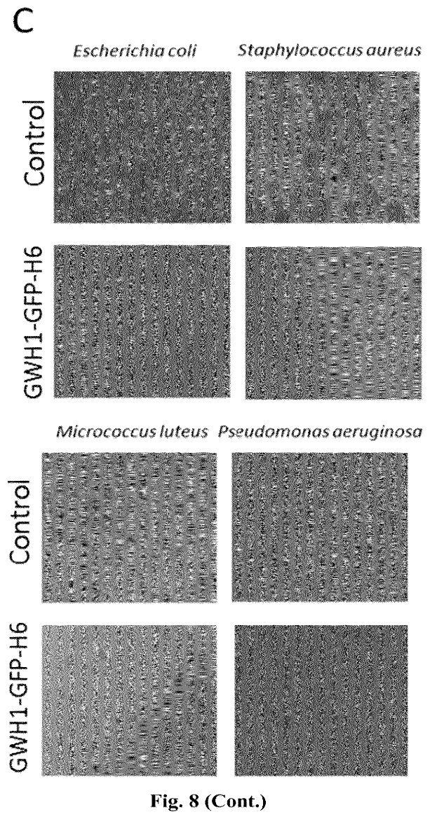

To test if GWH1-GFP-H6 would keep the antimicrobial activity upon assembly as protein nanoparticles we exposed cultures of several bacterial species to this material. As observed (FIG. 8 A), GWH1-GFP-H6 showed a potent antibiotic activity over three out of four species, that was clearly dose-dependent (FIG. 8B). The activity of the protein over P. aeruginosa was still evident but milder than in the rest of targets.

Bacterial death was in all cases clearly linked to cell lysis (FIG. 8C), strongly suggesting that the antimicrobial activity was reached by the conventional membrane activity of GWH1. Since it has not been previously described if a free AMP amino terminus is required for such activity (what happens in the case of other AMPs, and envisaging a future potential design of more complex GWH1-based recombinant constructs, we also tested T22-GWH1-GFP-H6 nanoparticles in the same assay. T22 is a cationic ligand of the cytokine receptor CXCR4, that might be clinically relevant to HIV infection (since this protein is a co-receptor of the virus) and to several human cancers such as pancreatic cancer, metastatic melanoma or osteosarcoma that overexpress this receptor [19-23]. As observed, T22-GWH1-GFP-H6 nanoparticles were still active over the target bacterial cells, although with a reduced efficiency (FIG. 8A). The antimicrobial properties of these materials were not provided by T22, as the related oligomeric construct T22-GFP-H6 did not showed any biological effect (FIG. 8A).

### Example 9: Cytotoxic Activities of GWH1-Based Protein Nanoparticles

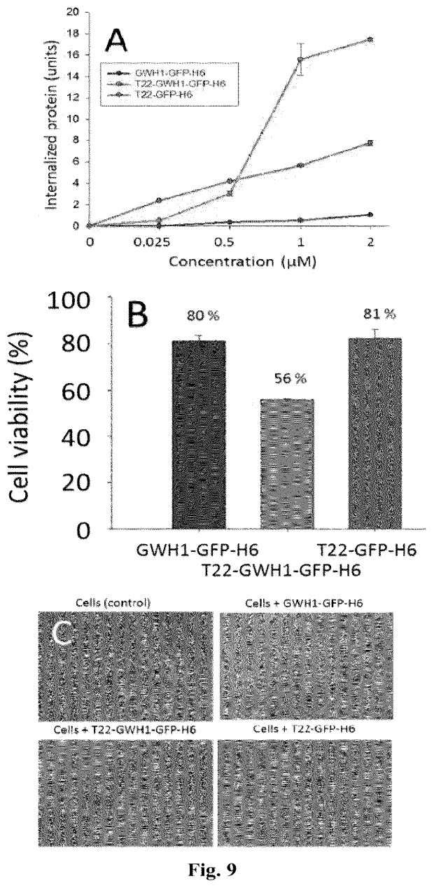

We were also interested in knowing if GWH1-GFP-H6 nanoparticles would also show cytotoxic potential. This is important since any residual anti-cellular activity of GWH1 nanoparticles would preclude their potential use as antimicrobials. Since GWH1-GFP-H6 exhibited a specific fluoresce that represented approximately 50% of that shown by a His-tagged GFP (not shown), we were able to monitor potential internalization in cultured human cells. As observed, GWH1-GFP-H6 did not internalize HeLa cells, while both related constructs carrying the CXCR4 ligand T22 were able to penetrate these cultured cells (FIG. 9A). GWH1-GFP-H6 nanoparticles were equally inefficient in promoting HeLa cell death, as it also occurred with the control T22-GFP-H6 (FIG. 9B). However, strong cytotoxicity was apparent in cells exposed to T22-GWH1-GFP-H6 nanoparticles (FIG. 9 B), indicating that the combination of an intracellular targeting agent (T22) and the AMP was efficient in cell killing.

### Example 10: Protein Nanoparticles Based on Diphtheria Toxin (DITOX) and Pseudomonas aeruginosa Exotoxin (PE24)

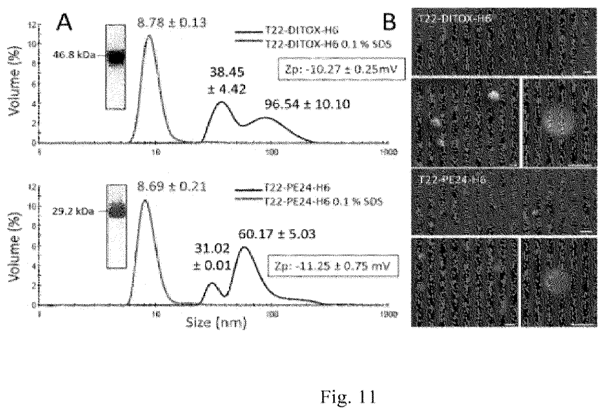

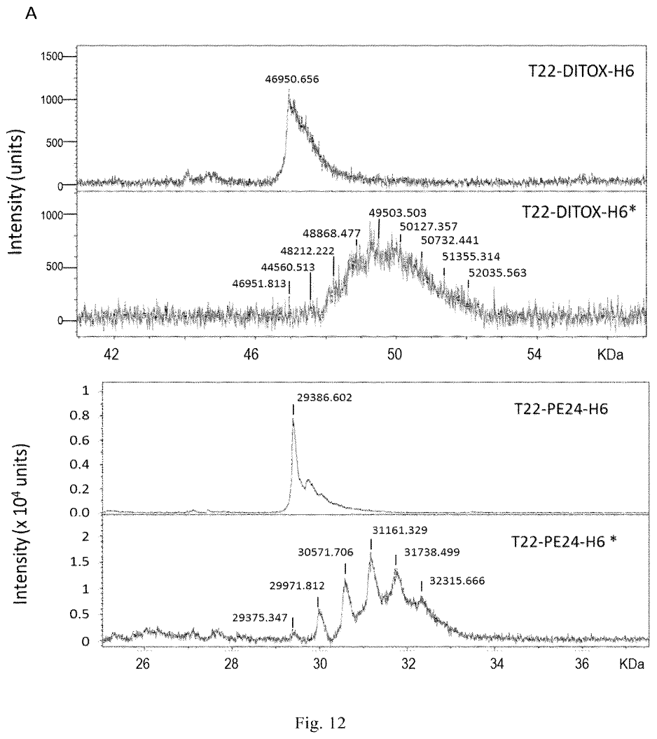

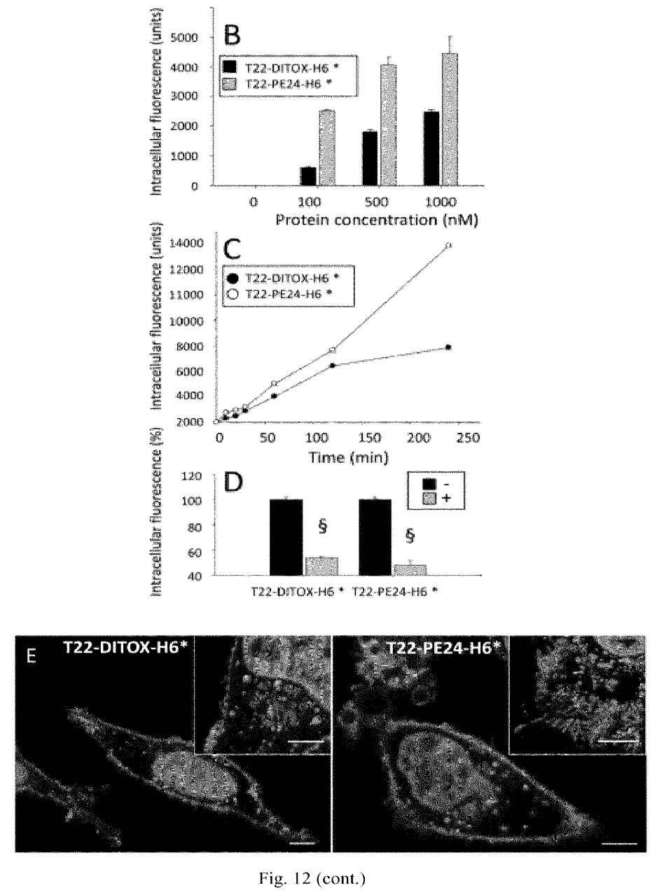

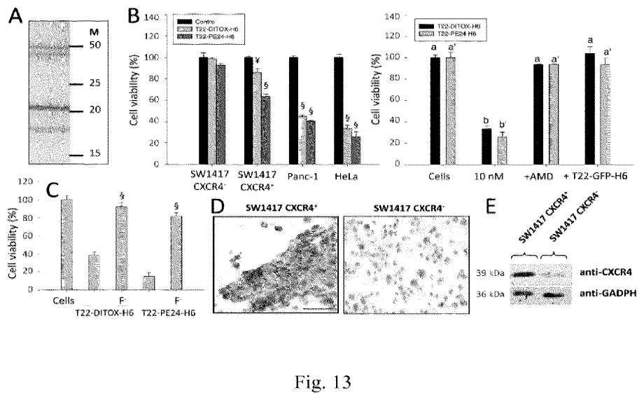

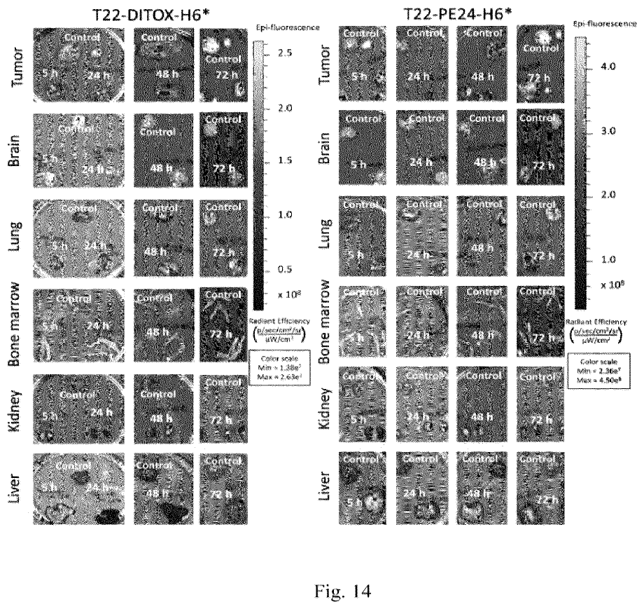

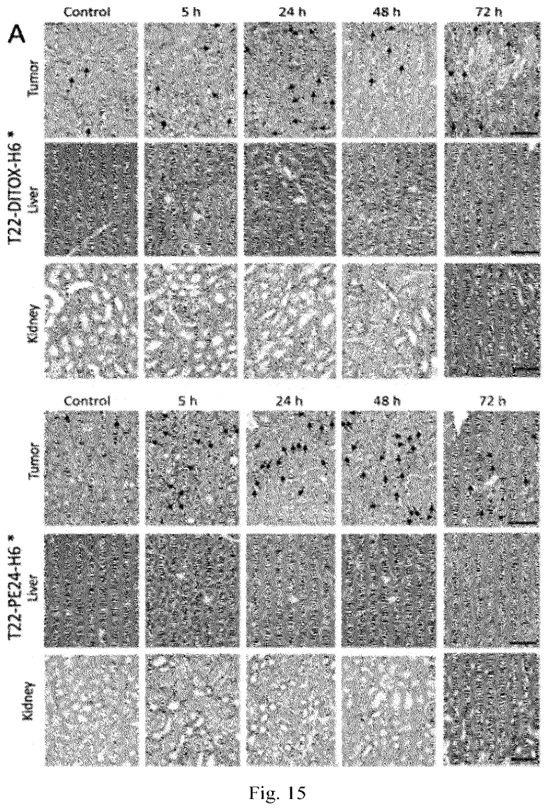

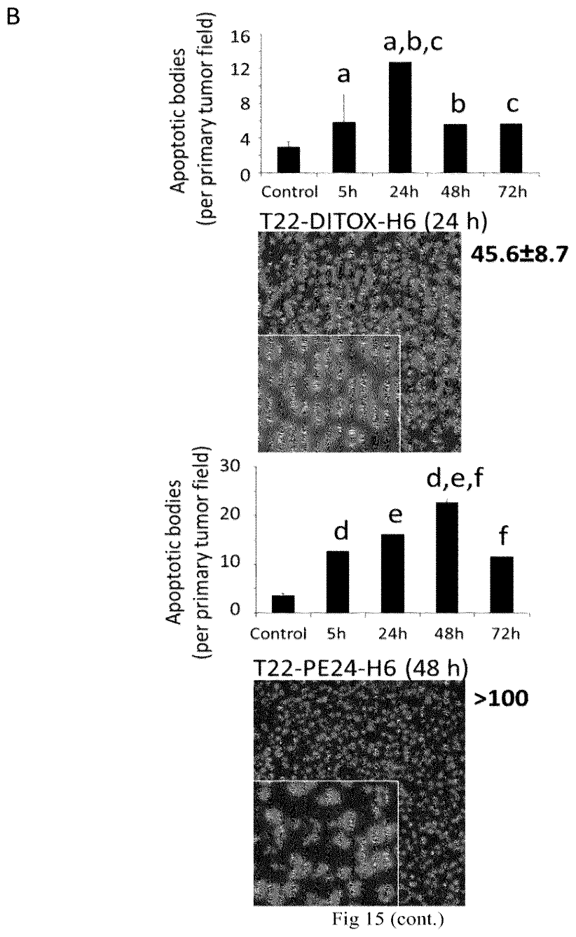

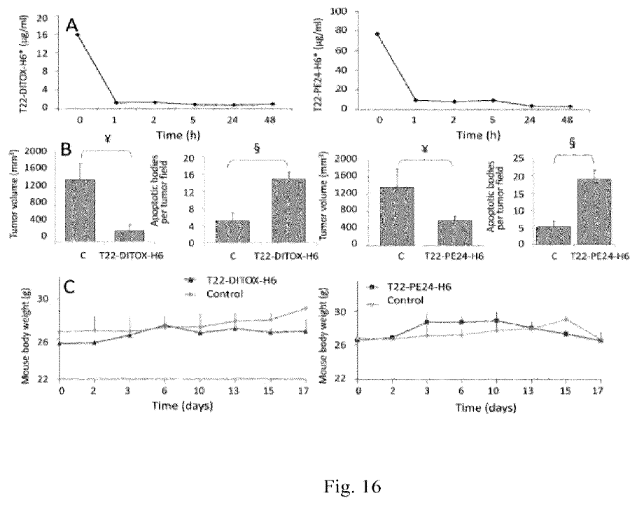

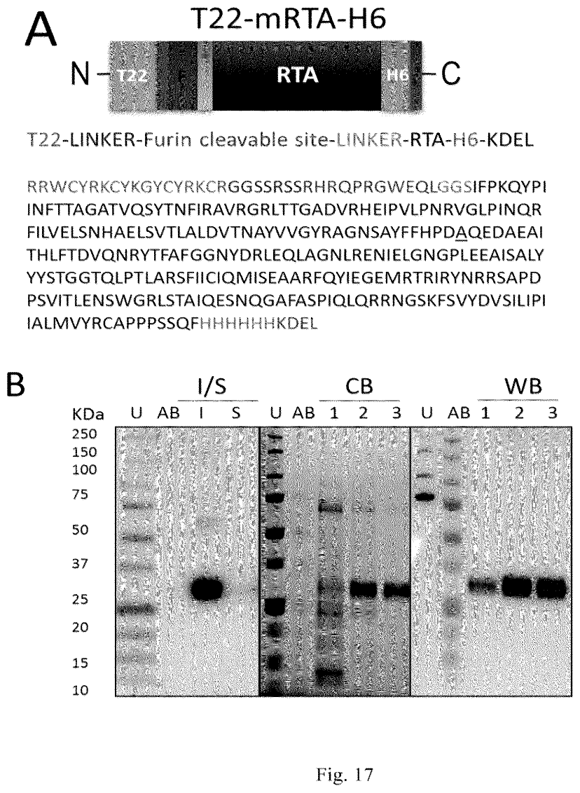

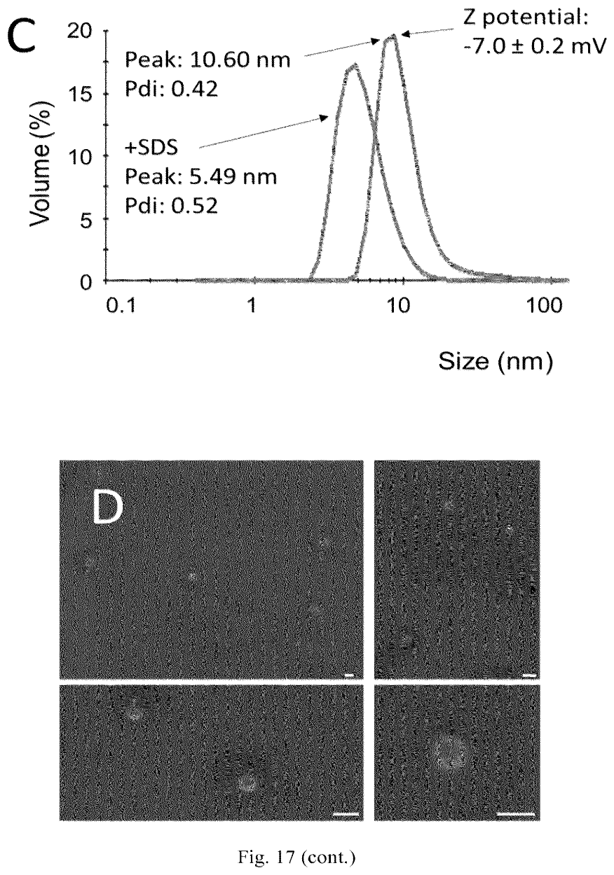

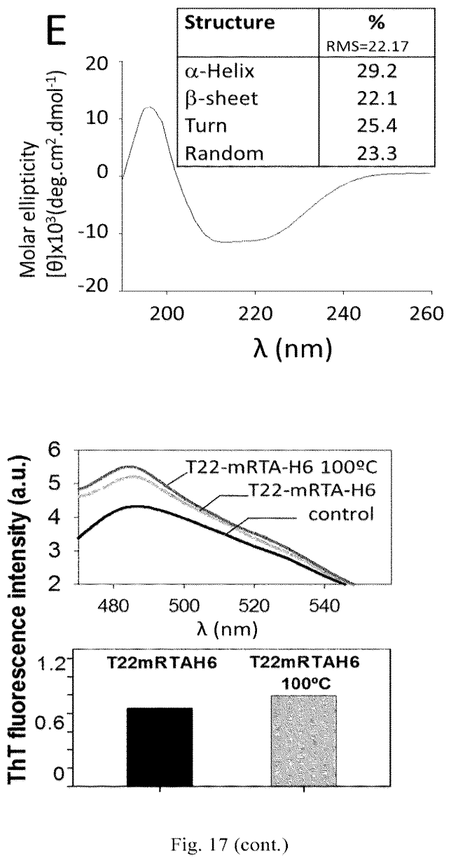

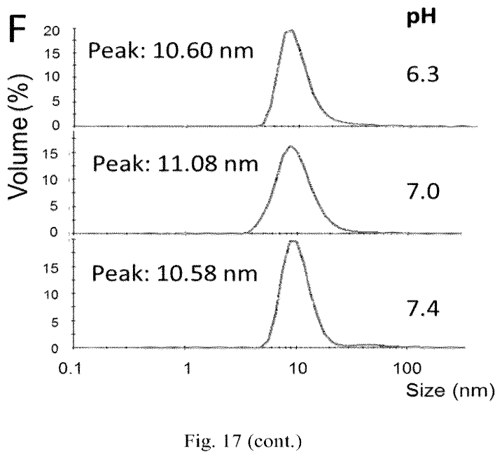

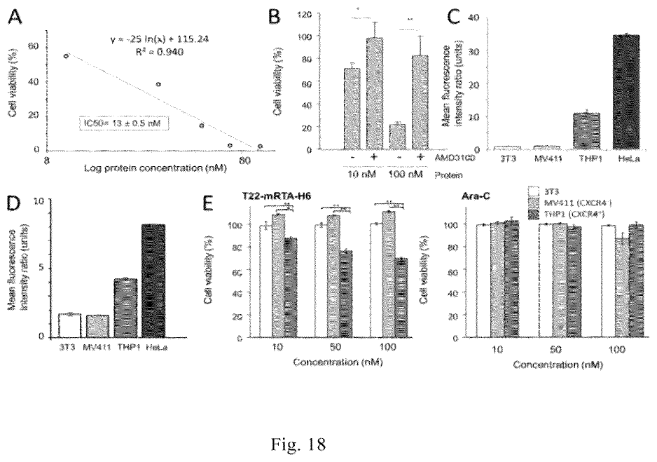

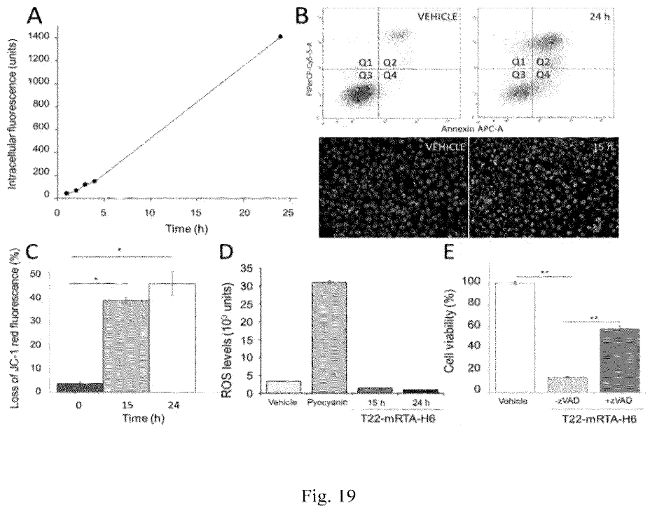

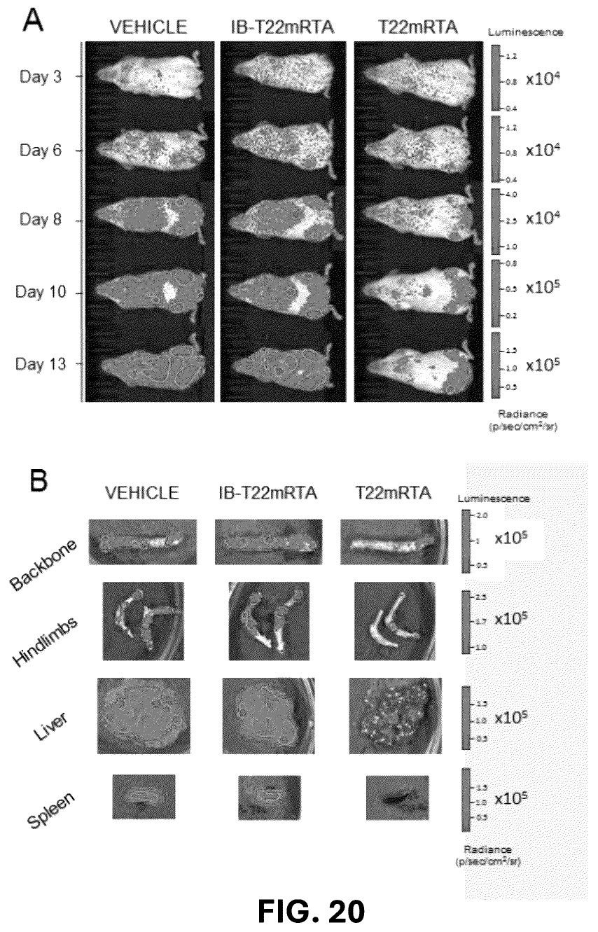

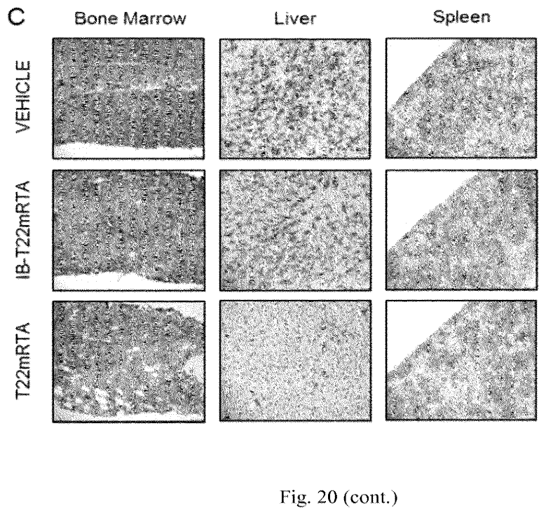

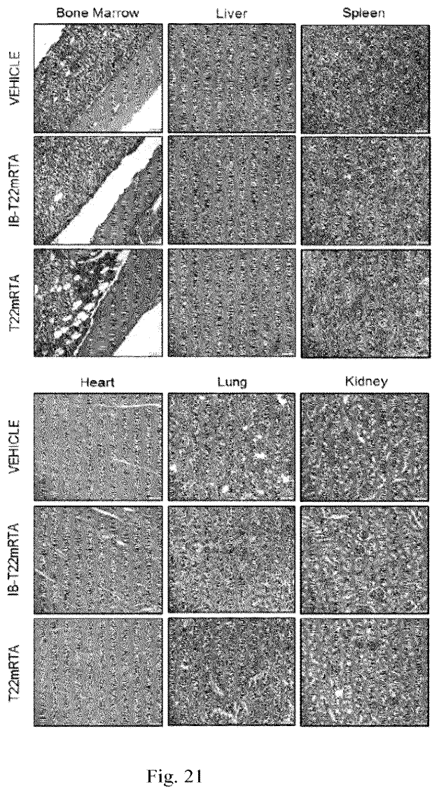

Active fragments of the diphtheria toxin (DITOX) and the Pseudomonas aeruginosa exotoxin (PE24) were produced in Escherichia coli as the modular fusion proteins T22-DITOX-H6 and T22-PE24-H6 (FIG. 10A, B), intended to induce targeted cell death through the activity of the catalytic fragments of the protein drug (FIG. 10C). The cationic peptide T22, placed at the amino terminus of the whole construct and cooperating with carboxy terminal histidines, promotes both oligomerization into regular nanoparticles and binding to the cell-surface chemokine receptor CXCR4 (overexpressed in many aggressive human cancers). In this way, it has been proved efficient in endorsing the endosomal penetration of payload GFP and IRFP into CXCR4+ cancer stem cells. Then, T22-DITOX-H6 spontaneously self-assembled into 38 and 90 nm-nanoparticles (Pdi=0.25±0.01 nm) and T22-PE24-H6 into ~60 nm-nanoparticles (Pdi=0.22±0.01, FIG. 11A), always within the size range considered as optimal for efficient cell uptake. A secondary population of protein material was observed in the case of T22-PE24-H6, being always minority. Nanoparticles were effectively disassembled by 0.1% SDS, resulting in monodisperse building blocks peaking at ~6 nm (Pdi=0.60±0.01 and 0.30±0.07 respectively), compatible with the expected size of the monomeric protein. However, both protein nanoparticles were fully stable in several physiological buffers in medium term incubation and also when exposed to high salt content buffer (up to 1M NaCl, not shown), what prompted us expecting high stability in vivo. In addition, nanoparticles were found stable after one year storage at −80° C. and upon repeated cycles of freezing and thawing (not shown). The assembled proteins appeared as toroid materials (FIG. 11B), with ultrastructural morphometry (round shape and clear size populations) that confirmed the size range observed by DLS. The same regular architecture had been previously described for the related T22-GFP-H6 construct, in which the GFP-based sub-units (with a molecular size similar to that of T22-DITOX-H6 and T22-PE24-H6) organized in toroid entities, whose organization has been modelled in silico and confirmed by sophisticated analytical methods such as SAXS or high resolution electron microscopy imaging techniques.

Purified T22-DITOX-H6 and T22-PE24-H6 nanoparticles were tested for internalization into cultured CXCR4+ cells, upon chemically labelling with the fluorescent dye ATTO 488 (tagged with *, FIG. 12A). Both kinds of labelled nanoparticles (FIG. 12A) penetrated target HeLa cells in a dose-dependent manner (FIG. 12B) and accumulated intracellularly with a kinetics characteristic of receptor-mediated uptake (with a faster slope in the case of T22-PE24-H6*, FIG. 12C). The CXCR4 specificity of the penetration was confirmed through its inhibition by the CXCR4 antagonist AMD3100 (FIG. 12D). Internalized nanoparticles were observed as engulfed into endosomes, especially in cytoplasm areas close to the cell membrane, but they tended to be visualized as membrane-free entities when approaching the perinuclear regions (FIG. 12E), suggesting important endosomal escape. No cell-attached extracellular fluorescence was observed in any case.

Once the internalization was assessed, we tested if the furin cleavage sites introduced in the constructs to release the toxin segments from the building blocks were active in the oligomers. The expected intracellular hydrolysis should enhance the cytotoxic properties of the toxin domains, which would then benefit from lower load of superfluous protein sequences. For that, we explored the sensitivity of the multiple cleavage sites in the construct T22-DITOX-H6 that would offer, upon intracellular digestion, fully distinguishable protein fragments. Unlike the extracellular protein that appears as one single protein species (FIGS. 11A and 12A), the His tag immunodetection of the cell-engulfed protein showed the protein as digested by different alternative sites, matching the molecular weight of the expected products for each furin cleavage site. In particular, the release of the T22 peptide through the de novo incorporated cleavage site was proved in vivo in cell-internalized protein by the shift from the 48.65 kDa full-length protein to the 44.21 kDa fragment, analyzing cell extracts upon exposure to the nanoparticles for 24 h (FIG. 13A). The rest of fragments corresponded to the progressive digestion intermediates that still kept the carboxy terminal tag, by which the protein is immunodetected. The natural cleavage at the internal furin site, which releases the catalytic domain from the translocation domain, is also proved by the occurrence of the major 20.60 kDa segment. Therefore, the catalytic segment alone is expected to occur inside the target cells, among other biologically active versions, at reasonable amounts.

When exploring the cytotoxic effects, both T22-DITOX-H6 and T22-PE24-H6 were effective in killing cultured HeLa cells, with low IC50 values (0.78 nM and 0.99 nM respectively, not shown). The cytotoxic effect was clearly detectable in several CXCR4-expressing cell lines, including SW1417 CXCR4+ but not in the isogenic SW1417 CXCR4-line (FIG. 13B, left). Cytotoxicity was mostly abolished by AMD3100 and by the T22-displaying biologically inner protein T22-GFP-H6 (FIG. 13B, right), thus confirming again the specificity of the entrance of the nanoparticles, the intracellular nature of the nanoparticle-mediated toxicity and the expected CXCR4 receptor mediation in cell killing. Besides, it has been observed a reversion effect of T22-DITOX-H6 (90%) when adding chloroquine, which inhibits endosomal acidification (not shown). This fact confirms that the mechanism of action is pH-dependent as described above (FIG. 10). In this context, the relevance of the removal of accessory protein segments (mediated by furin) on the cytotoxicity of the nanoparticles was also evaluated. For that, versions of T22-DITOX-H6 and T22-PE24-H6 without the engineered cleavage sites (labelled as F-) were constructed and tested for biological activity. The comparative analyses of HeLa cell death mediated by these proteins revealed a dramatic drop of cytotoxicity in T22-DITOX-H6 F- and T22-PE24-H6 F-nanoparticles compared to the original materials (FIG. 13 C). On the other hand, the differential CXCR4 expression in the isogenic SW1417 cells was fully assessed by immunocytochemistry and Western blot (FIG. 13 D,E). Interestingly, the capacity of T22-DITOX-H6 and T22-PE24-H6 to promote cell death was not lost after one-year storage at −80° C. and also upon 4 cycles of freezing and thawing (not shown). Due to the high CXCR4+ specific cytotoxicity observed in cell culture, we next tested the performance of the toxin-based materials in vivo using a CXCR4-linked disease model. For that, we explored the biodistribution, antitumor activity and potential side toxicity of both T22-DITOX-H6* and T22-PE24-H6* nanoparticles in a CXCR4 overexpressing subcutaneous colorectal cancer model. As expected, after a single dose i.v. administration the protein materials accumulated in tumor in the studied time range (FIG. 14). Other organs such as brain, lung or heart were completely free of fluorescence. However, significant levels of emission associated to both nanoparticles were found in liver and kidney. To discard that significant amounts of ATTO might had been released from the nanoparticles during circulation in blood and generate artefacts in the biodistribution analysis, we evaluated the stability of the dye in T22-DITOX-H6* nanoparticles incubated in commercial serum. At 48 h, only a very minor fraction of fluorescence was released from nanoparticles (5%). In addition, the administration of free ATTO did not resulted in detectable accumulation in tumor, and the absence of dye signal in major organs was indicative of a fast urine secretion (as expected for a small molecule of 981 Da). These data fully supported the biodistribution of labelled nanoparticles shown in FIG. 14.

The presence of nanoparticles in liver was observed as worthy of a deeper analysis, since hepatic occurrence and damage is a severe concern in conventional and innovative cancer therapies, even in nanoconjugates or antibody-based drugs that show tissue-specific targeting. Then, since it would be of crucial interest to discriminate between mere occurrence of fluorescence and toxin-induced damage in these organs, we comparatively investigated cell damage in tumor, liver and kidney. In this regard, we observed a high level of apoptosis induced by both nanoparticles in tumoral tissue, what was especially intense in T22-PE24-H6*-treated animals at 48 h post administration (FIG. 15). In contrast, apoptosis was undetectable in liver or kidney (FIG. 15), and most of the hepatic tissues were histologically normal except for a few and scattered small inflammation foci (FIG. 15) that can be attributed to non-specific extracellular retention of the drug in off target tissues. This alteration was resolved after 72 h returning to normal histology. Probably, the intracellular activation of the toxins promoted by the furin-mediated release of accessory peptides (FIG. 13) does not occur in hepatic tissue, which does not overexpress CXCR4. To discard that ATTO might have a positive contribution in the cytotoxicity of the materials in tumor after single dose administration we checked local apoptosis in animals treated with the non-labelled protein versions T22-DITOX-H6 and T22-PE24-H6. This was done at the times, among those tested, showing the highest potency (24 and 48 h respectively). As observed, local apoptosis was still present (FIG. 15) at values even higher than those induced by the labelled protein versions. This result was indicative that the observed antitumoral effect was intrinsically associated to the protein material. Then, data supported the notion that in spite of the occurrence of the protein drug in liver and kidney, this did not translate in a relevant uptake of any of the two nanoparticles in the parenchyma of these tissues. Our observations suggest that both labelled protein drugs underwent transient circulation through the fenestrated hepatic sinusoids and renal glomeruli despite their nanometric size (in contrast to other normal tissues) as reported for other nanoparticles. Moreover, their lack of toxicity in kidney or liver suggest their inability to internalize into the parenchymal cells in these organs because of their negligible CXCR4 expression, in comparison for instance to spleen or bone marrow which despite showing low nanoparticle accumulation express CXCR4. This is a finding similar to that reported for CXCR4-targeted imaging agents. Then, both T22-DITOX-H6 and T22-PE24-H6 appear to have a therapeutic index high enough to validate (i) their potential use for the treatment of CXCR4+ tumors but more importantly, and (ii) the wide applicability of the transversal concept supporting the self-assembling self-driving protein drugs based on chemically homogenous building blocks. In order to evaluate further the therapeutic potential of the engineered toxins and the concepts that support the design of toxin-based nanoparticles we also assessed the pharmacokinetics in blood mouse samples after a single dose of 50 μg for T22-DITOX-H6* or 300 μg for T22-PE24-H6*. This was done through registering their fluorescence emission at 0, 1, 2, 5, 24 and 48 h after administration. We observed a biphasic decline in plasma concentration from Cmax, with a fast nanoparticle biodistribution limited to the plasma compartment for both tested proteins (Vd=3.9 ml T22-PE-H6* and Vd=3.2 ml for T22-DITOX-H6*). This fast biodistribution was followed by a second and slow elimination phase, with a half-life of t½=30 h for both nanoparticles (FIG. 16A). This kinetic behavior is similar to the one previously reported for pharmacologically inactive protein nanoparticles, and also similar to that described for antibody-drug conjugates or large nanometric size therapeutic proteins, which show a comportment similar to the unconjugated antibody. In a step further, we assessed the antitumor effect of each nanoparticle in a CXCR4+ subcutaneous CRC mouse model after repeated dose administration. After a dosage schedule of 10 μg of T22-DITOX-H6, three times a week, per 8 doses, we observed at the end of the experiment a 5.8-fold reduction in tumor volume, as compared to buffer-treated mice (p=0.05). This was associated with a 3.0-fold increase in apoptotic figures in tumor tissue (p<0.001) (FIG. 16B), with no significant differences in body weight between toxin-treated and control groups (FIG. 16C). Similarly, and after a dosage schedule in mice of 10 μg of T22-PE24-H6, three times a week, per 8 doses, we observed at the end of the experiment a 2.3-fold reduction in tumor volume, as compared to buffer-treated mice (p=0.034), associated with a 3.8-fold increase in the number of apoptotic figures in tumor tissue (p=0.001) (FIG. 16B). Again, no significant differences in body weight between experimental and control groups were observed (FIG. 16C).

### Example 11: Protein Nanoparticles Based on Recombinant Ricin (mRTA)

The recombinant T22-mRTA-H6 (FIG. 17A) was successfully produced in Escherichia coli Origami B, purified by His-based one-step affinity chromatography and detected as a single protein species with the expected molecular mass of 35.91 kDa (FIG. 17B), that was fully confirmed by mass spectrometry (not shown). The pure protein was straightforward observed by both, DLS and FESEM, as ~11 nm entities occurring in the storage buffer without further treatment (FIG. 17C, D), indicating the spontaneous formation of self-assembled nanoparticles. This was the expected outcome as the combination of cationic peptides at the amino terminus and polyhistidines at the carboxy terminus has been proved to be optimal to promote protein oligomerization as regular nanostructures, irrespective of the core protein segment (ricin, in the case of T22-mRTA-H6, FIG. 17A). Treating the material with SDS resulted in monomers of 5.5 nm (FIG. 17C), which represented the probable building blocks of the nanoparticles. In the related self-assembling protein T22-GFP-H6, in which the sizes of the building block and the assembled version are both equivalent to those of T22-mRTA-H6, the use of small-angle X-ray scattering and other sophisticated analytical methods as well as in silico modelling have revealed that the nanoparticle was formed by approximately 10 monomers. Being estimative, this figure fits also to T22-mRTA-H6. The analysis of T22-mRTA-H6 nanoparticles by circular dichroism (CD) revealed a structural composition in which α-helix predominates (29.2%, FIG. 17 E). However, a Thioflavin T (Th T) assay has also revealed the occurrence of intermolecular β-sheet interactions (FIG. 17 F) that might contribute to the stability of protein nanoparticles, and that is also compatible with the extent of important 3-sheet structure found in the CD (FIG. 17 E). Since the nanostructured ricin was intended to be delivered in tumoral tissues, we wondered if the nanoparticles could be still stable in the abnormal pH values observed in the tumor environment, that have been reported to range from approximately 6.3 (intracellular) to 7.4 (extracellular). As observed, T22-mRTA-H6 remained fully assembled under these conditions (FIG. 17 F), what supports the usability of construct from the stability point of view.

In order to test the functionality of the recombinant ricin in such assembled form, cultured CXCR4+ HeLa cells were exposed to different concentrations of ricin-based nanoparticles. These materials showed a potent, dose-dependent cytotoxicity that essentially abolished cell viability at 100 nM (FIG. 18 A). After 72 h of exposure, the IC50 was determined to be 13±0.5 nM. To confirm if, as expected, T22-mRTA-H6-mediated cell death was dependent on its cell binding and internalization of the protein via the cell surface receptor CXCR4 and its ligand T22, we tested if a potent CXCR4 antagonist, AMD3100, could be able to recover cell viability when used as a competitor of the toxin, at a molar ratio of 10:1. As observed (FIG. 18 B), AMD3100 dramatically enhanced cell viability in T22-mRTA-H6-treated cells proving a specific, receptor-mediated penetration of the nanoparticles into target cells. To further confirm such precision cell entry mechanism, we decided to exposed non tumoral (CXCR4−) 3T3 cells and representative CXCR4− and CXCR4+ tumoral cell lines to T22-mRTA-H6, and also to a conventional chemical drug used in the treatment of several cancer types but specially of acute myeloid leukemia (AML), namely cytosine arabinoside (Ara-C). These cell lines, with different levels of CXCR4 expression (FIG. 18 C), supported different levels of protein internalization mediated by the specific interaction between T22 and CXCR4 (FIG. 18 D). This was determined through the uptake of T22-GFPH6, a self-assembling fluorescent protein closely related to T22-mRTA-H6 that contains the same ligand of CXCR4 also accommodated at the amino terminus of the polypeptide. It must be noted that as predicted, CXCR4 expression and T22-mediated protein internalization showed a parallel behavior (compare FIG. 18 C and D). Then, when they were finally comparatively tested, the ricin-based protein nanoparticle promoted specific cell death only in CXCR4+ cancer cells but not in normal cells, at a dose (100 nM) at which Ara-C did not show any toxic effect on any of these cell lines (FIG. 18 E). This observation proved not only the effective targeting of the protein drug but also its superior cytotoxicity compared to an equimolar dose of the model chemical drug.

At this stage, we wanted to confirm that the cytotoxicity promoted by T22-mRTA-H6 was linked to the uptake of the nanoparticles inside CXCR4+ cells, and triggered from within. This was reached by exposing HeLa cells to ATTO-labelled nanoparticles and monitoring internalization. As observed (FIG. 19 A), nanoparticles were internalized by cells at least up to 24 h. As expected for an active version of ricin, apoptosis was detected though both annexin affinity assay and by Hoechst staining (FIG. 19 B), and the number of apoptotic cells seemed to peak at around 15-24 h post exposure. In addition, mitochondrial damage was confirmed by the significant increase in the number of cells with lowered JC-1 red fluorescence at 15 and 24 h after treatment with T22-mRTA-H6 (FIG. 19 C), indicative of a depolarization in the mitochondrial ΔΨ linked to apoptotic induction. Interestingly, cell damage occurred without a detectable increase in reactive oxygen species (ROS, FIG. 19 D), while the formation of apoptotic bodies in ricin-exposed HeLa cells was clearly caspase-dependent (FIG. 19 E). The combination of these data indicates that T22-mRTA-H6-mediated cell death occurs by a classical caspase-bddependent apoptosis pathway.

The antitumor effect of both T22-mRTA-H6 soluble nanoparticles and T22-mRTA-H6 IBs was evaluated in a disseminated AML animal model. NSG mice were injected with THP1-Luci cells to generate leukemia dissemination in mice. Two days after cell injection through the vein tail, a single dose injection was performed in the mice hypodermis (SC) of 1 mg of T22-mRTA-H6 IBs in two mice (IB-T22mRTA group). In a different mouse group, daily intravenous administrations were started of 10 μg of soluble T22-mRTA-H6 (T22mRTA group) to one mouse or buffer alone (VEHICLE group) to three mice, for a total of 10 doses. No effects on mice weight were observed during the treatments (data not shown). The progression and dissemination of leukemia was assessed by monitoring BLI using the IVIS Spectrum. From the day 6 and until the end of the experiment, the mouse treated with soluble T22-mRTA-H6 (T22mRTA) showed lower luminescence emission than the VEHICLE group (FIG. 20A). Thus, as measured by BLI, treatment with soluble T22-mRTA-H6 inhibited the dissemination of AML cells in mice, compared to the vehicle group, after the 4th, 6th, 8th and 10th doses of T22-mRTA-H6 at 10 μg per dose (which corresponded to day 6, 8, 10 or 13 after injection of cells, respectively). In contrast, no differences in BLI were found between mice treated with T22-mRTA-H6 IBs (IB-T22mRTA) and the control VEHICLE mice (FIG. 20A).

In a next step, the antitumor activity of nanoparticles was analyzed in affected organs ex vivo 14 days after the injection of cells when mice presented signs of advanced disease. The analyses with the IVIS Spectrum showed that the treatment with soluble T22-mRTA-H6 nanoparticles (T22mRTA) decreased BLI in the bone marrow (backbone and hindlimbs), liver and spleen, in contrast to the findings in mice treated with buffer alone (VEHICLE) (FIG. 21B). However, the treatment with T22-mRTA-H6 IBs (IB-T22mRTA) did not show changes in BLI in the same tissues in comparison to control mice (VEHICLE) (FIG. 20B).

In addition, the dissemination of leukemic cells was evaluated in the affected organs of the animal by IHC of CD45, a human leukocyte marker that detects AML THP1 cells. Results correlated with BLI analyses showing that treatment with soluble T22-mRTA-H6, differently from those registered after T22-mRTA-H6 IBs treatment, reduced the dissemination in the infiltrated tissues, by detecting lower number of CD45 positive cells in bone marrow, liver and spleen in the mouse treated with soluble T22-mRTA-H6 (FIG. 20C). Finally, H&E staining was performed of the infiltrated organs and additional organs not affected by leukemia cells. No sign of toxicity in any of the affected or unaffected tissues, neither with the soluble T22-mRTA-H6 nor with the T22-mRTA-H6 IBs treatments were observed (FIG. 21). As it occurred in vitro, IBs caused, if any, just a mild biological effect.

1-57. (canceled)

58. A method of treating a cancer comprising administering a nanoparticle to a subject in need thereof wherein the nanoparticle comprises a plurality of polypeptides comprising (i) a polycationic peptide that specifically binds to a CXCR4 receptor, (ii) a cytotoxic polypeptide, and (iii) a polyhistidine, wherein the polypeptides self-assemble to form the nanoparticle, wherein the cancer comprises cancer cells expressing a CXCR4 receptor, wherein the nanoparticle binds to the CXCR4 receptor, wherein the nanoparticle is internalized by the cancer cells expressing the CXCR4 receptor, and wherein the internalization of the nanoparticle triggers the death of the cancer cells expressing the CXCR4 receptor.

59. The method of claim 1, wherein the cancer is selected from the group consisting of pancreatic cancer, colorectal cancer, a central nervous system cancer, head and neck cancer, leukemia, and lymphoma.


SEQUENCE LISTING (48 sequences)

SEQ ID NO: 1 - protein, 9 aa, synthetic construct
  REGION 1..9: note = Polycationic peptide 1
RRRRRRRRR

SEQ ID NO: 2 - protein, 9 aa, synthetic construct
  REGION 1..9: note = Polycationic peptide 2
RRRGRGRRR

SEQ ID NO: 3 - protein, 9 aa, synthetic construct
  REGION 1..9: note = Polycationic peptide 3
RARGRGRRR

SEQ ID NO: 4 - protein, 9 aa, synthetic construct
  REGION 1..9: note = Polycationic peptide 4
RARGRGGGA

SEQ ID NO: 5 - protein, 18 aa, Limulus polyphemus
RRWCYRKCYKGYCYRKCR

SEQ ID NO: 6 - protein, 21 aa, Herpesvirus
LGASWHRPDKCCLGYQKRPLP

SEQ ID NO: 7 - protein, 67 aa, Homo sapiens
KPVSLSYRCPCRFFESHVARANVKHLKILNTPNCALQIVARLKNNNRQVCIDPKLKWIQEYLEKALN

SEQ ID NO: 8 - protein, 68 aa, Herpesvirus
LGASWHRPDKCCLGYQKRPLPQVLLSSWYPTSQLCSKPGVIFLTKRGRQVCADKDWVKKLMQQLPVTA

SEQ ID NO: 9 - protein, 14 aa, synthetic construct
  REGION 1..14: note = T140 peptide
  MOD_RES 3: note = X = L-3-(2-naphthyl)alanine
  MOD_RES 8: note = X = D-Lys
  MOD_RES 12: note = X = L-Citrulline
RRXCYRKXPYRXCR

SEQ ID NO: 10 - protein, 14 aa, synthetic construct
  REGION 1..14: note = TN14003 peptide
  MOD_RES 3: note = X = L-3-(2-naphthyl)alanine
  MOD_RES 6: note = X = L-Citrulline
  MOD_RES 8: note = X =D-Lys
  MOD_RES 12: note = X =L-Citrulline
RRXCYXKXPYRXCR

SEQ ID NO: 11 - protein, 14 aa, synthetic construct
  REGION 1..14: note = TC 14012 peptide
  MOD_RES 3: note = X = L-3-(2-naphthyl)alanine
  MOD_RES 8: note = X = D-Citrulline
  MOD_RES 12: note = X = L-Citrulline
RRXCYEKXPYRXCR

SEQ ID NO: 12 - protein, 14 aa, synthetic construct
  REGION 1..14: note = TE14011 peptide
  MOD_RES 3: note = X = L-3-(2-naphthyl)allanine
  MOD_RES 6: note = X = L-Citrulline
  MOD_RES 8: note = X = D-Glutamic acid
  MOD_RES 12: note = X = L-Citrulline
RRXCYXKXPYRXCR

SEQ ID NO: 13 - protein, 14 aa, synthetic construct
  REGION 1..14: note = TZ14011 peptide
  MOD_RES 3: note = X = L-3-(2)-naphthyl)alanine
  MOD_RES 6: note = X = L-Citrulline
  MOD_RES 8: note = X = D-Lysine
  MOD_RES 12: note = X = L-Citrulline
RRXCYXKXPYRXCR

SEQ ID NO: 14 - protein, 20 aa, synthetic construct
  REGION 1..20: note = GW-H1 peptide
GYNYAKKLANLAKKFANALW

SEQ ID NO: 15 - protein, 13 aa, Homo sapiens
RLVSYNGIIFFLK

SEQ ID NO: 16 - protein, 69 aa, Homo sapiens
WQPPRARITGYIIKYEKPGSPPREVVPRPRPGVTEATITGLEPGTEYTIYVIALKNNQKSEPLIGRKKT

SEQ ID NO: 17 - protein, 18 aa, synthetic construct
  REGION 1..18: note = Seq-1-7 peptide
KYLAYPDSVHIWRKRKRK

SEQ ID NO: 18 - protein, 19 aa, synthetic construct
  REGION 1..19: note = Seq-1-8 peptide
KYLAYPDSVHIWRKRKRKR

SEQ ID NO: 19 - protein, 22 aa, synthetic construct
  REGION 1..22: note = Angiopep-2-7 peptide
TFFYGGSRGKRNNFKTEEYRKR

SEQ ID NO: 20 - protein, 39 aa, synthetic construct
  REGION 1..39: note = Nucleolin-binding peptide (F3-RK peptide)
KDEPQRRSARLSAKPAPPKPEPKPKKAPAKKRKRKRKRK

SEQ ID NO: 21 - protein, 14 aa, synthetic construct
  REGION 1..14: note = Linker 1
SGGTSGSTSGTGST

SEQ ID NO: 22 - protein, 15 aa, synthetic construct
  REGION 1..15: note = Linker 2
AGSSTGSSTGPGSTT

SEQ ID NO: 23 - protein, 7 aa, synthetic construct
  REGION 1..7: note = Linker 3
GGSGGAP

SEQ ID NO: 24 - protein, 8 aa, synthetic construct
  REGION 1..8: note = Linker 4
GGGVEGGG

SEQ ID NO: 25 - protein, 10 aa, synthetic construct
  REGION 1..10: note = Linker 5
PKPSTPPGSS

SEQ ID NO: 26 - protein, 11 aa, synthetic construct
  REGION 1..11: note = Linker 6
APAETKAEPMT

SEQ ID NO: 27 - protein, 5 aa, synthetic construct
  REGION 1..5: note = Cleavage site 1 (enterokinase)
DDDDK

SEQ ID NO: 28 - protein, 5 aa, synthetic construct
  REGION 1..5: note = Cleavage site 2 (factor Xa)
IEDGR

SEQ ID NO: 29 - protein, 6 aa, synthetic construct
  REGION 1..6: note = Cleavage site 3 (thrombin)
LVPRGS

SEQ ID NO: 30 - protein, 7 aa, synthetic construct
  REGION 1..7: note = Cleavage site 4 (TEV protease)
ENLYFQG

SEQ ID NO: 31 - protein, 8 aa, synthetic construct
  REGION 1..8: note = Cleavage site 5 (PreScission protease)
LEVLFQGP

SEQ ID NO: 32 - protein, 7 aa, synthetic construct
  REGION 1..7: note = Preferred linker 1
GGSSRSS

SEQ ID NO: 33 - protein, 5 aa, synthetic construct
  REGION 1..5: note = Preferred linker 2
GGGNS

SEQ ID NO: 34 - protein, 211 aa, Homo sapiens
MASGQGPGPPRQECGEPALPSASEEQVAQDTEEVFRSYVFYRHQQEQEAEGVAAPADPEMVTLPLQPSSTMGQVGRQLAIIGDDINRRYDSEFQTMLQHLQPTAENAYEYFTKIATSLFESGINWGRVVALLGFGYRLALHVYQHGLTGFLGQVTRFVVDFMLHHCIARWIAQRGGWVAALNLGNGPILNVLVVLGVVLLGQFVVRRFFKS

SEQ ID NO: 35 - protein, 15 aa, Homo sapiens
VGRQLAIIGDDINRR

SEQ ID NO: 36 - protein, 193 aa, Homo sapiens
MARARQEGSSPEPVEGLARDGPRPFPLGRLVPSAVSCGLCEPGLAAAPAAPTLLPAAYLCAPTAPPAVTAALGGSRWPGGPRSRPRGPRPDGPQPSLSLAEQHLESPVPSAPGALAGGPTQAAPGVRGEEEQWAREIGAQLRRMADDLNAQYERRRQEEQQRHRPSPWRVLYNLIMGLLPLPRGHRAPEMEPN

SEQ ID NO: 37 - protein, 187 aa, Corynebacterium diphtheriae [sequence omitted]

SEQ ID NO: 38 - protein, 209 aa, Pseudomonas aeruginosa
GDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 39 - protein, 8 aa, synthetic construct
  REGION 1..8: note = Linker sequence
GGSSRSSS

SEQ ID NO: 40 - protein, 25 aa, Homo sapiens
STMGQVGRQLAIIGDDINRRYDSEF

SEQ ID NO: 41 - protein, 25 aa, Homo sapiens
EEQWAREIGAQLRRMADDLNAQYER

SEQ ID NO: 42 - protein, 7 aa, synthetic construct
  REGION 1..7: note = Linker sequence
GGSSRSS

SEQ ID NO: 43 - protein, 382 aa, Corynebacterium diphtheriae
GADDWDSSKSFVMENFSSYHGTKPGYVDSIQKGIQKPKSGTQGNYDDDWKGFYSTDNKYDAAGYSVDNENPLSGKAGGWKVTYPGLTKVLALKVDNAETIKKELGLSLTEPLMEQVGTEEFIKRFGDGASRWLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQWHNSYNRPAYSPGHKT

SEQ ID NO: 44 - protein, 218 aa, synthetic construct
  REGION 1..218: note = peptide PE24 based on de-immunized catalytic domain of Pseudomonas aeruginosa exotoxin A
PTGAEFLGDGGDVSFSTRGTQNWTVERLLQAHAQLEERGYVFVGYHGTFLEAAQSIVFGGVAARSQDLAAIWAGFYIAGDPALAYGYAQDQEPDAAGRIRNGALLRVYVPASSLPGFYRTSLTLAAPEAAGEVERLIGHPLPLALDAITGPEEEGGRLETILGWPLAERTWIPSAIPTDPRNVGGDLDPSSIPDKEQAISALPDYASQPGKPPREDLK

SEQ ID NO: 45 - protein, 267 aa, synthetic construct
  REGION 1..267: note = Chain A from Ricin from the seeds of Ricinus communis, with the mutation N132A
IFPKQYPIINFTTAGATVQSYTNFIRAVRGRLTTGADVRHEIPVLPNRVGLPINQRFILVELSNHAELSVTLALDVTNAYVVGYRAGNSAYFFHPDAQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQF

SEQ ID NO: 46 - protein, 8 aa, synthetic construct
  REGION 1..8: note = Target cleavage site for the furin protease
GNRVRRSV

SEQ ID NO: 47 - protein, 11 aa, synthetic construct
  REGION 1..11: note = Target cleavage site for the furin protease
RHRQPRGWEQL

SEQ ID NO: 48 - protein, 4 aa, synthetic construct
  REGION 1..4: note = KDEL endosomal escape peptide
KDEL